SEQUENCE LISTING (37 sequences)

SEQ ID NO: 1 - DNA, 318 bp, Escherichia coli
atggctgaatggagcggcgaatatatcagcccatacgctgagcacggcaagaagagtgaacaagtcaaaaagattacggtttccattcctcttaaggtgttaaaaatcctcaccgatgaacgcacgcgtcgtcaggtgaacaacctgcgtcacgctaccaacagcgagctgctgtgcgaagcgtttctgcatgcctttaccgggcaacctttgccggatgatgccgatctgcgtaaagagcgcagcgacgaaatcccggaagcggcaaaagagatcatgcgtgagatggggattaacccggagacgtgggaatactaa

SEQ ID NO: 2 - DNA, 462 bp, Escherichia coli
gtgagcagagtaaccgcgattatatccgctctgattatctgcatcatcgtcagcctgtcatgggcggtcaatcattaccgtgataacgcaatcgcctacaaagtccagcgcgacaaaaatgccagagaactgaagctagcgaacgcggcaattactgacatgcagatgcgtcagcgtgatgttgctgcgctcgatgcaaaatacacgaaggagttagctgatgcgaaagctgaaaatgatgctctgcgtgatgatgttgccgctggtcgtcgtcggttgcacatcaaagcagtctgtcagtcagtgcgtgaagccaccacggcctccggcgtggataatgcagcctccccccgactggcagacaccgctgaacgggattatttcaccctcagagagaggctgatcactatgcaaaaacaactggaaggaacccagaagtatattaatgagcagtgcagatag

SEQ ID NO: 3 - DNA, 1398 bp, Escherichia coli
atgaaaatggtctcacgtattaccgcgatcggcctggctggcgtcgcgatttgctatttagggttatctggttatgtgtggtaccacgataataaacgcagtaaacaggccgatgttcaggcatctgctgtcagtgaaaataataaggttttaggctttctccgcgaaaaaggatgcgactattgccacacgccttcggcagaattacccgcctattattatattcctggcgcgaaacagttgatggattacgacattaagcttggatataaatcttttaaccttgaggccgtgcgtgcggcactgctggctgataaacccgtttcgcaaagcgatttgaataagattgaatgggtgatgcagtatgaaactatgccaccaacgcgttataccgcgctacactgggcgggtaaggtgagtgatgaagagcgggcggaaatactggcctggattgcaaaacagcgcgcggaatattacgccagcaatgatactgctccggaacatcgcaatgaaccggtgcagcccatcccgcaaaaactgcctaccgatgcgcaaaaagtggcgttgggttttgcgctgtatcacgatccccgtttatcggctgatagcaccatttcatgcgctcattgccatgcgttgaatgcggggggcgtcgatggcagaaaaacatcgattggtgttggtggcgcagttgggccgattaacgcgccgacggtatttaactcagtatttaacgttgagcagttctgggatggtcgtgcggcaacattgcaggatcaggctggtggaccgccgttgaacccgattgaaatggcgtcgaaatcctgggacgaaattattgctaagctggaaaaagatccgcagcttaaaacgcagttcctcgaagtctatccgcaaggtttcagtggcgaaaatattactgatgccattgctgaatttgagaaaacattaattacgccggattccccatttgataaatggttgcgcggagatgaaaatgcgctgacggcgcaacagaaaaaaggctatcaattatttaaagataataaatgtgcaacttgtcatggtggtattattctcggcggacgttcctttgaaccgttggggctgaaaaaagactttaactttggggaaattacggcggcggatattggtcgtatgaatgtgactaaagaagagcgtgataaattgcgtcagaaagtacccggtttacgtaacgttgctttaacggcaccgtacttccatcgcggtgacgtgccgacgctggacggggcggtgaaactgatgctgcgctatcaggtaggcaaagagctgccgcaggaggatgtggatgatatcgtagctttcctgcacagtctgaacggggtgtacacgccgtatatgcaggataaacaataa

SEQ ID NO: 4 - DNA, 1332 bp, Escherichia coli
atgaataaagcaataaaagtatcattgtatatatcttttgttttgattatttgcgccttatctaaaaacataatgatgttaaatacatctgatttcggaagagccattaagccattaattgaagacataccagcatttacatatgacttacctttattgtataaattgaaaggtcatattgattcaattgatagctatgagtatataagttcatatagttatattttgtatacatacgtcctgtttattagcatttttactgaatatcttgatgctagggtgttatcgttatttctaaaagtaatatatatttattcattatatgcgatatttacttcatatataaaaacagaaaggtatgtaactttatttacattctttattttagcttttcttatgtgttcttcatcaacactgtcaatgtttgcatcattctatcaagagcaaatagttataattttccttccatttttggtgtattcattaacatgcaaaaacaataaatctatgcttttgctatttttttcgttgctaataatatctactgctaaaaatcaatttatattaaccccactaatagtgtattcatattatattttttttgatagacacaaactaattattaaatctgtaatatgcgtggtgtgcttgcttgcgtcaatatttgcaatatcttattcaaaaggtgttgttgaattaaataagtaccatgcaacatacttcggtagttatctttatatgaaaaacaacgggtataaaatgccatcgtatgttgatgataagtgtgttgggttagatgcctggggtaataaattcgacatatcatttggcgcaaccccaacagaagttggaacggaatgtttcgaatctcataaagatgaaacgttttcgaatgcactctttttattggttagcaaaccaagcaccatcttcaaacttccatttgatgatggtgtgatgtctcagtataaagaaaattatttccatgtatataaaaaactacacgtaatatatggagaatcaaacatactaacgactattactaacataaaagacaatatatttaaaaacattagatttatatcattgttattattttttattgcttctatttttattagaaataataaaataaaggcatctttatttgtagtatctctttttggaatatctcaattttatgtgtcatttttcggggaaggatatagagatttaagcaagcatttatttggaatgtatttttcgttcgacctttgcttatacataacagtcgtttttttaatttataaaataattcaaagaaatcaagacaatagcgatgtaaagcactaa

SEQ ID NO: 5 - DNA, 987 bp, Escherichia coli
atgattcctgattatttaacttttattcgctttcaggataaacgaaatctgatatacatttatgctattggacttattctgataggcttttattggaagaatgcagggtttacttttccatcagaggatattggtgtagttagtgggattctggctctggtgctgtataattttatttttgatctcaaggcgtactgggcttataaatgcgtcacgaagaatatcgatttttcgtggtttaagaaaaagcagaaccacaaaatagaattatttcttacacaacctctggtggcaggatttctgtcgttaatcatgttgagtgcaatgagttgggggctataccagcttctaccctcgttatatgcgctgttcctgatttcgttacttgggccgttggtcatctttctgctgtttcggatgatccgcaccagttatgtcaagcaggtcgctatttcagtagcgaaaaaagtaaaatataaaagtctgactcgctatgtgctgctttcggtgtgcatctcaacggttgttaacctgcttactatcagcccgttgcgtaacagtgattcttttgtgacagaggggcagtggttaacgtttaaatcgataattgcattgctcattctttgtggcgtagtgttggcgattaatctgttttttctgcgcttctccaagcggtacgcttttctgggcaggctttttttgcaggaaatcgatctgtttttctccagtgaaaatgcgttgtcgaccttttttgccaagccgctttggcttcggttattcatattgctggttattgaagtgatgtggattacgctggtgtcggtattggcaacgcttgtagaatggcggatttggtttgaagcctattttttactctgctatgtaccgtgcttaatttactattttttctattgtcgattcctctggcataacgattttatgatggcatgtgacatgtatttccgttgggggcattttaataagtga

SEQ ID NO: 6 - DNA, 489 bp, Escherichia coli
atgagactgacatctaaagggcgctatgccgtgaccgcaatgcttgacgttgcgctcaactctgaagcgggcccggtaccgttggctgatatttccgaacgtcagggaatttccctttcttatctggaacaactgttttcccgtctgcgtaaaaatggtctggtttccagcgtacgtggaccaggcggtggttatctgttaggcaaagatgccagcagcatcgccgttggcgaagtaattagcgccgttgacgaatctgtagatgccacccgttgtcagggtaaaggcggctgccagggcggcgataaatgcctgacccacgcgctgtggcgtgatttgagcgaccgtctcaccggttttctcaacaacattactttaggcgaactggttaataaccaggaagtgctggatgtgtctggtcgtcagcatactcacgacgcgccacgcacccgcacacaagacgcgatcgacgttaagttacgcgcttaa

SEQ ID NO: 7 - DNA, 639 bp, Escherichia coli
atggctgtcgctgccaacaaacgttcggtaatgacgctgttttccggtcctactgacatctatagccatcaggtccgcattgtgctggctgagaaaggtgtaagtttcgagatcgaacacgtggaaaaggacaatccgcctcaggatctgattgacctcaacccgaatcagagcgttccgaccctggtggatcgtgagctgaccctgtgggaatctcgcatcattatggaatatctggatgagcgtttcccgcatccgccactgatgcctgtttacccggtagctcgcggtgaaagccgtctgtacatgcatcgcatcgaaaaagactggtacacgctgatgaacaccatcatcaacggttcagcttctgaagcagatgccgcacgtaagcaactgcgcgaagaactgctggcgattgcgccggtcttcggtcagaagccgtacttcctgagcgatgagttcagcctggtcgattgctatcttgctccgctgctgtggcgtctgccgcaactgggcatcgagttcagcggcccgggtgcgaaagagctgaaaggctatatgacccgcgtctttgagcgtgactctttccttgcttctttaactgaagcagaacgtgaaatgcgtctgggccggagttaa

SEQ ID NO: 8 - DNA, 716 bp, Escherichia coli
atgcgtccagcaggccgtagcaataatcaggtgcgtcccgttaccctgactcgtaactatacaaaacatgcagaaggctcggtgctggtcgaatttggcgataccaaagtgttgtgtaccgcctctattgaagaaggcgtgccgcgcttcctgaaaggtcagggccagggctggatcaccgcagagtacggcatgctgccacgttctacccacacccgtaacgctcgtgaagcggcgaaaggtaagcagggtggacgcacaatggaaatccagcgtctgatcgcccgtgctcttcgcgcggcagtagatttgaaagcgctgggtgagttcaccattacgctggactgcgacgtgcttcaggctgatggtggcacgcgtaccgcgtcgattacgggtgcctgcgtggcgctggtagatgcgctacagaagctggtggaaaacggcaagctgaaaaccaatccgatgaaagggatggtagccgcagtttctgtcggaattgtgaacggcgaagcggtttgcgatctggaatacgttgaagactctgccgcagagaccgacatgaacgtagtgatgaccgaagacgggcgcatcattgaagtgcaggggacggcagaaggcgagccgttcacccatgaagagctactcatcttgttggctctggcccgaggggaatcgaatccattgtagcgacgcagaaggcggcgctggcaaactga

SEQ ID NO: 9 - DNA, 2235 bp, Escherichia coli
atggttgcggtaagaagtgcacatatcaataaggctggtgaatttgatccggaaaaatggatcgcaagtctgggtattaccagccagaagtcgtgtgagtgcttagccgaaacctgggcgtattgtctgcaacagacgcaggggcatccggatgccagtctgttattgtggcgtggtgttgagatggtggagatcctctcgacattaagtatggacattgacacgctgcgggcggcgctgcttttccctctggcggatgccaacgtagtcagcgaagatgtgctgcgtgagagcgtcggtaagtcggtcgttaaccttattcacggcgtgcgtgatatggcggcgatccgccagctgaaagcgacgcacactgattctgtttcctccgaacaggtcgataacgttcgccggatgttattggcgatggtcgatgattttcgctgcgtagtcatcaaactggcggagcgtattgctcatctgcgcgaagtaaaagatgcgccggaagatgaacgtgtactggcggcaaaagagtgtaccaacatctacgcaccgctggctaaccgtctcggaatcggacaactgaaatgggaactggaagattactgcttccgttacctccatccaaccgaatacaaacgaattgccaaactgctgcatgaacggcgtctcgaccgcgaacactacatcgaagagttcgttggtcatctgcgcgctgagatgaaagctgaaggcgttaaagcggaagtgtatggtcgtccgaaacacatctacagcatctggcgtaaaatgcagaaaaagaacctcgcctttgatgagctgtttgatgtgcgtgcggtacgtattgtcgccgagcgtttacaggattgctatgccgcactggggatagtgcacactcactatcgccacctgccggatgagtttgacgattacgtcgctaacccgaaaccaaacggttatcagtctattcataccgtggttctggggccgggtggaaaaaccgttgagatccaaatccgcaccaaacagatgcatgaagatgcagagttgggtgttgctgcgcactggaaatataaagagggcgcggctgctggcggcgcacgttcgggacatgaagaccggattgcctggctgcgtaaactgattgcgtggcaggaagagatggctgattccggcgaaatgctcgacgaagtacgtagtcaggtctttgacgaccgggtgtacgtctttacgccgaaaggtgatgtcgttgatttgcctgcgggatcaacgccgctggacttcgcttaccacatccacagtgatgtcggacaccgctgcatcggggcaaaaattggcgggcgcattgtgccgttcacctaccagctgcagatgggcgaccagattgaaattatcacccagaaacagccgaaccccagccgtgactggttaaacccaaacctcggttacgtcacaaccagccgtgggcgttcgaaaattcacgcctggttccgtaaacaggaccgtgacaaaaacattctggctgggcggcaaatccttgacgacgagctggaacatctggggatcagcctgaaagaagcagaaaaacatctgctgccgcgttacaacttcaatgatgtcgacgagttgctggcggcgattggtggcggggatatccgtctcaatcagatggtgaacttcctgcaatcgcaatttaataagccgagtgccgaagagcaggacgccgccgcgctgaagcaacttcagcaaaaaagctacacgccgcaaaaccgcagtaaagataacggtcgcgtggtagtcgaaggtgttggcaacctgatgcaccacatcgcgcgctgctgccagccgattcctggagatgagattgtcggcttcattacccaggggcgcggtatttcagtacaccgcgccgattgcgaacaactggcggaactgcgctcccatgcgccagaacgcattgttgacgcggtatggggtgagagctactccgccggatattcgctggtggtccgcgtggtagctaatgatcgtagtgggttgttacgtgatatcacgaccattctcgccaacgagaaggtgaacgtgcttggcgttgccagccgtagcgacaccaaacagcaactggcgaccatcgacatgaccattgagatttacaacctgcaagtgctggggcgcgtgctgggtaaactcaaccaggtgccggatgttatcgacgcgcgtcggttgcacgggagttag

SEQ ID NO: 10 - DNA, 1179 bp, Escherichia coli
atgacgttattaggcactgcgctgcgtccggcagcaactcgcgtgatgttattaggctccggtgaactgggtaaagaagtggcaatcgagtgtcagcgtctcggcgtagaggtgattgccgtcgatcgctatgccgacgcaccagccatgcatgtcgcgcatcgctcccatgtcattaatatgcttgatggtgatgcattacgccgtgtggttgaactggaaaaaccacattatatcgtgccggagatcgaagctattgccaccgatatgctgatccaacttgaagaggaaggactgaatgttgtcccctgcgctcgcgcaacgaaattaacgatgaatcgcgagggtatccgtcgcctggcggcagaagagctgcagctgcccacttccacttatcgttttgccgatagcgaaagccttttccgcgaggcggttgctgacattggctatccctgcattgtaaaaccggtgatgagctcttccggcaaggggcagacgtttattcgttctgcagagcaacttgctcaggcatggaagtacgctcagcaaggcggtcgcgccggagcgggccgcgtaattgttgaaggcgtcgttaagtttgacttcgaaattaccctgctaaccgtcagcgcggtggatggcgtccatttctgtgcaccagtaggtcatcgccaggaagatggcgactaccgtgaatcctggcaaccacagcaaatgagcccgcttgcccttgaacgtgcgcaggagattgcccgtaaagtggtgctggcactgggcggttatgggttgtttggtgtcgagctatttgtctgtggtgatgaggtgattttcagtgaggtctcccctcgtccacatgataccgggatggtgacgttaatttctcaagatctctcagagtttgccctgcatgtacgtgccttcctcggacttccggttggcgggatccgtcagtatggtcctgcagcttctgccgttattctgccacaactgaccagtcagaatgtcacgtttgataatgtgcagaatgccgtaggcgcagatttgcagattcgtttatttggtaagccggaaattgatggcagccgtcgtctgggggtggcactggctactgcagagagtgttgttgacgccattgaacgcgcgaagcacgccgccggacaggtaaaagtacagggttaa

SEQ ID NO: 11 - DNA, 3150 bp, Escherichia coli
atgcctaatttctttatcgatcgcccgatttttgcgtgggtgatcgccattatcatcatgttggcaggggggctggcgatcctcaaactgccggtggcgcaatatcctacgattgcaccgccggcagtaacgatctccgcctcctaccccggcgctgatgcgaaaacagtgcaggacacggtgacacaggttatcgaacagaatatgaacggtatcgataacctgatgtacatgtcctctaacagtgactccacgggtaccgtgcagatcaccctgacctttgagtctggtactgatgcggatatcgcgcaggttcaggtgcagaacaaactgcagctggcgatgccgttgctgccgcaagaagttcagcagcaaggggtgagcgttgagaaatcatccagcagcttcctgatggttgtcggcgttatcaacaccgatggcaccatgacgcaggaggatatctccgactacgtggcggcgaatatgaaagatgccatcagccgtacgtcgggcgtgggtgatgttcagttgttcggttcacagtacgcgatgcgtatctggatgaacccgaatgagctgaacaaattccagctaacgccggttgatgtcattaccgccatcaaagcgcagaacgcccaggttgcggcgggtcagctcggtggtacgccgccggtgaaaggccaacagcttaacgcctctattattgctcagacgcgtctgacctctactgaagagttcggcaaaatcctgctgaaagtgaatcaggatggttcccgcgtgctgctgcgtgacgtcgcgaagattgagctgggtggtgagaactacgacatcatcgcagagtttaacggccaaccggcttccggtctggggatcaagctggcgaccggtgcaaacgcgctggataccgctgcggcaatccgtgctgaactggcgaagatggaaccgttcttcccgtcgggtctgaaaattgtttacccatacgacaccacgccgttcgtgaaaatctctattcacgaagtggttaaaacgctggtcgaagcgatcatcctcgtgttcctggttatgtatctgttcctgcagaacttccgcgcgacgttgattccgaccattgccgtaccggtggtattgctcgggacctttgccgtccttgccgcctttggcttctcgataaacacgctaacaatgttcgggatggtgctcgccatcggcctgttggtggatgacgccatcgttgtggtagaaaacgttgagcgtgttatggcggaagaaggtttgccgccaaaagaagctacccgtaagtcgatggggcagattcagggcgctctggtcggtatcgcgatggtactgtcggcggtattcgtaccgatggccttctttggcggttctactggtgctatctatcgtcagttctctattaccattgtttcagcaatggcgctgtcggtactggtggcgttgatcctgactccagctctttgtgccaccatgctgaaaccgattgccaaaggcgatcacggggaaggtaaaaaaggcttcttcggctggtttaaccgcatgttcgagaagagcacgcaccactacaccgacagcgtaggcggtattctgcgcagtacggggcgttacctggtgctgtatctgatcatcgtggtcggcatggcctatctgttcgtgcgtctgccaagctccttcttgccagatgaggaccagggcgtgtttatgaccatggttcagctgccagcaggtgcaacgcaggaacgtacacagaaagtgctcaatgaggtaacgcattactatctgaccaaagaaaagaacaacgttgagtcggtgttcgccgttaacggcttcggctttgcgggacgtggtcagaataccggtattgcgttcgtttccttgaaggactgggccgatcgtccgggcgaagaaaacaaagttgaagcgattaccatgcgtgcaacacgcgctttctcgcaaatcaaagatgcgatggttttcgcctttaacctgcccgcaatcgtggaactgggtactgcaaccggctttgactttgagctgattgaccaggctggccttggtcacgaaaaactgactcaggcgcgtaaccagttgcttgcagaagcagcgaagcaccctgatatgttgaccagcgtacgtccaaacggtctggaagataccccgcagtttaagattgatatcgaccaggaaaaagcgcaggcgctgggtgtttctatcaacgacattaacaccactctgggcgctgcatggggcggcagctatgtgaacgactttatcgaccgcggtcgtgtgaagaaagtttatgtcatgtcagaagcgaaataccgtatgctgccggatgatatcggcgactggtatgttcgtgctgctgatggtcagatggtgccattctcggcgttctcctcttctcgttgggagtacggttcgccgcgtctggaacgttacaacggcctgccatccatggaaatcttaggccaggcggcaccgggtaaaagtaccggtgaagcaatggagctgatggaacaactggcgagcaaactgcctaccggtgttggctatgactggacggggatgtcctatcaggaacgtctctccggcaaccaggcaccttcactgtacgcgatttcgttgattgtcgtgttcctgtgtctggcggcgctgtacgagagctggtcgattccgttctccgttatgctggtcgttccgctgggggttatcggtgcgttgctggctgccaccttccgtggcctgaccaatgacgtttacttccaggtaggcctgctcacaaccattgggttgtcggcgaagaacgcgatccttatcgtcgaattcgccaaagacttgatggataaagaaggtaaaggtctgattgaagcgacgcttgatgcggtgcggatgcgtttacgtccgatcctgatgacctcgctggcgtttatcctcggcgttatgccgctggttatcagtactggtgctggttccggcgcgcagaacgcagtaggtaccggtgtaatgggcgggatggtgaccgcaacggtactggcaatcttcttcgttccggtattctttgtggtggttcgccgccgctttagccgcaagaatgaagatatcgagcacagccatactgtcgatcatcattga

SEQ ID NO: 12 - DNA, 1194 bp, Escherichia coli
atgaacaaaaacagagggtttacgcctctggcggtcgttctgatgctctcaggcagcttagccctaacaggatgtgacgacaaacaggcccaacaaggtggccagcagatgcccgccgttggcgtagtaacagtcaaaactgaacctctgcagatcacaaccgagcttccgggtcgcaccagtgcctaccggatcgcagaagttcgtcctcaagttagcgggattatcctgaagcgtaatttcaaagaaggtagcgacatcgaagcaggtgtctctctctatcagattgatcctgcgacctatcaggcgacatacgacagtgcgaaaggtgatctggcgaaagcccaggctgcagccaatatcgcgcaattgacggtgaatcgttatcagaaactgctcggtactcagtacatcagtaagcaagagtacgatcaggctctggctgatgcgcaacaggcgaatgctgcggtaactgcggcgaaagctgccgttgaaactgcgcggatcaatctggcttacaccaaagtcacctctccgattagcggtcgcattggtaagtcgaacgtgacggaaggcgcattggtacagaacggtcaggcgactgcgctggcaaccgtgcagcaacttgatccgatctacgttgatgtgacccagtccagcaacgacttcctgcgcctgaaacaggaactggcgaatggcacgctgaaacaagagaacggcaaagccaaagtgtcactgatcaccagtgacggcattaagttcccgcaggacggtacgctggaattctctgacgttaccgttgatcagaccactgggtctatcaccctacgcgctatcttcccgaacccggatcacactctgctgccgggtatgttcgtgcgcgcacgtctggaagaagggcttaatccaaacgctattttagtcccgcaacagggcgtaacccgtacgccgcgtggcgatgccaccgtactggtagttggcgcggatgacaaagtggaaacccgtccgatcgttgcaagccaggctattggcgataagtggctggtgacagaaggtctgaaagcaggcgatcgcgtagtaataagtgggctgcagaaagtgcgtcctggtgtccaggtaaaagcacaagaagttaccgctgataataaccagcaagccgcaagcggtgctcagcctgaacagtccaagtcttaa

SEQ ID NO: 13 - DNA, 648 bp, Escherichia coli
atgggcgtaagagcgcaacaaaaagaaaaaacccgccgttcgctggtggaagccgcatttagccaattaagtgctgaacgcagcttcgccagcctgagtttgcgtgaagtggcgcgtgaagcgggcattgctcccacctctttttatcggcatttccgcgacgtagacgaactgggtctgaccatggttgatgagagcggtttaatgctacgccaactcatgcgccaggcgcgtcagcgtatcgccaaaggcgggagtgtgatccgcacctcggtctccacatttatggagttcatcggtaataatcctaacgccttccggttattattgcgggaacgctccggcacctccgctgcgtttcgtgccgccgttgcgcgtgaaattcagcacttcattgcggaacttgcggactatctggaactcgaaaaccatatgccgcgtgcgtttactgaagcgcaagccgaagcaatggtgacaattgtcttcagtgcgggtgccgaggcgttggacgtcggcgtcgaacaacgtcggcaattagaagagcgactggtactgcaactgcgaatgatttcgaaaggggcttattactggtatcgccgtgaacaagagaaaaccgcaattattccgggaaatgtgaaggacgagtaa

SEQ ID NO: 14 - DNA, 2244 bp, Escherichia coli
atgaaactgaatgcaacttatataaaaatacgtgataaatggtgggggcttccgctgttcctgccttctttaatcttgcccattttcgcccacattaatactttcgcgcatatttcttccggtgaggtttttctcttttatctgcctctggcactgatgatcagcatgatgatgtttttcagctgggcggcattgccagggatcgccttagggatttttgtccgcaaatatgcagagctgggtttttacgaaacgctatcattaacggctaattttattatcattatcattctctgttggggcggttacagggtctttactccccggcgtaacaacgtttcacatggtgatacccgtttaatttcccagcgtatattctggcagattgtgtttcctgcaacgctgtttctgatacttttccagtttgctgcatttgtaggattactggcgagcagagaaaatctggtcggtgtcatgccttttaacctcgggactttaatcaattatcaggccttactggtgggtaatctgatcggtgtcccgctgtgctacttcatcattcgggtagtgcgaaatccattttatttacgtagctattattcgcaattaaaacagcaggttgatgccaaagtcaccaaaaaagagttcgcgctctggctactggcattaggtgctttattgttgctgttatgcatgccgttaaatgaaaaaagcacaatttttagcaccaattataccttgtcattattgctgcccctgatgatgtggggagcgatgcgctatggttataagctgatttcgctgctctgggcggtcgtgttgatgatcagcatccacagctatcaaaattacattcccatttatcctggctataccacgcagctgaccataacctcctccagttatctggtattctcttttattgtcaattatatggctgtactggcaacccgtcagagagcggtagtcagacgcattcagcggcttgcgtatgtggacccggtggttcatctgccaaatgttcgcgccctgaatcgcgcgttgcgtgatgccccctggtctgcgctttgttatttacgcatccctggcatggaaatgctggttaagaactatggcatcatgctgcggattcaatacaagcaaaaactttctcactggctgtcacccttgctggaaccgggtgaagatgtttatcagctttcgggtaacgatctcgcgctgcgactgaatacagaatcgcaccaggagcgcattaccgcactggatagccatctcaagcaatttcgtttcttttgggatggcatgccgatgcaaccgcagattggcgtcagttactgctatgtgcgctcgccagtgaatcatatctacctgctgctgggagagctaaatacggtcgccgaactttccatcgtgaccaacgccccggaaaatatgcagcgtcgcggggcaatgtatttgcaacgcgaattgaaagataaagtcgcgatgatgaatcgactacagcaggcgctggaacacaaccattttttcctgatggcccagccgattaccggtatgcgtggtgatgtttaccatgaaattcttctgcgcatgaaaggtgagaatgatgaactgatcagccccgatagcttcttgccggtcgcgcacgaatttggtttatcgtcgagtatcgacatgtgggtcattgagcatacgctgcaatttatggctgaaaacagagcgaagatgcccgctcaccgttttgctattaatctgtctccaacctcggtatgtcaggctcgttttcctgttgaagtcagtcagttgctggctaaatatcagattgaagcgtggcaacttatttttgaagtcaccgaaagtaatgctctgaccaatgttaagcaggcgcaaatcaccttgcagcatcttcaggaattaggctgccagattgcgattgatgatttcggcaccggctacgccagctatgcgcggcttaaaaatgtgaatgccgatctgcttaaaattgacggcagttttatccgcaatattgtgtcaaatagtctggattatcagatagtggcatcgatttgccacctggcgcgaatgaagaaaatgctggtagtggcagagtacgttgaaaacgaagagatccgcgaggcggtgctctctttggggatcgattatatgcagggttatcttattggtaagccgcagccgttaattgatacgctgaatgaaatcgaacccattcgcgaaagtgcctga

SEQ ID NO: 15 - DNA, 642 bp, Escherichia coli
atgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaa

SEQ ID NO: 16 - protein, 213 aa, Escherichia coli
MKPYQRQFIEFALSKQVLKFGEFTLKSGRKSPYFFNAGLFNTGRDLALLGRFYAEALVDSGIEFDLLFGPAYKGIPIATTTAVALAEHHDLDLPYCFNRKEAKDHGEGGNLVGSALQGRVMLVDDVITAGTAIRESMEIIQANGATLAGVLISLDRQERGRGEISAIQEVERDYNCKVISIITLKDLIAYLEEKPEMAEHLAAVKAYREEFGV

SEQ ID NO: 17 - DNA, 65 bp, Eschericha coli
gccttcgctcctcatcttacttttctacagacaaaaaaaaggcgactcatcagtcgccttaaaaa

SEQ ID NO: 18 - DNA, 876 bp, Escherichia coli
atgaccacactggcgattgatatcggcggtactaaacttgccgccgcgctgattggcgctgacgggcagatccgcgatcgtcgtgaacttcctacgccagccagccagacaccagaagccttgcgtgatgccttatccgcattagtctctccgttgcaagctcatgcgcagcgggttgccatcgcttcgaccgggataatccgtgacggcagcttgctggcgcttaatccgcataatcttggtggattgctacactttccgttagtcaaaacgctggaacaacttaccaatttgccgaccattgccattaacgacgcgcaggccgcagcatgggcggagtttcaggcgctggatggcgatataaccgatatggtctttatcaccgtttccaccggcgttggcggcggtgtagtgagcggctgcaaactgcttaccggccctggcggtctggcggggcatatcgggcatacgcttgccgatccacacggcccagtctgcggctgtggacgcacaggttgcgtggaagcgattgcttctggtcgcggcattgcagcggcagcgcagggggagttggctggcgcggatgcgaaaactattttcacgcgcgccgggcagggtgacgagcaggcgcagcagctgattcaccgctccgcacgtacgcttgcaaggctgatcgctgatattaaagccacaactgattgccagtgcgtggtggtcggtggcagcgttggtctggcagaagggtatctggcgctggtggaaacgtatctggcgcaggagccagcggcatttcatgttgatttactggcggcgcattaccgccatgatgcaggtttacttggggctgcgctgttggcccagggagaaaaattatga

SEQ ID NO: 19 - protein, 291 aa, Escherichia coli
MTTLAIDIGGTKLAAALIGADGQIRDRRELPTPASQTPEALRDALSALVSPLQAHAQRVAIASTGIIRDGSLLALNPHNLGGLLHFPLVKTLEQLTNLPTIAINDAQAAAWAEFQALDGDITDMVFITVSTGVGGGVVSGCKLLTGPGGLAGHIGHTLADPHGPVCGCGRTGCVEAIASGRGIAAAAQGELAGADAKTIFTRAGQGDEQAQQLIHRSARTLARLIADIKATTDCQCVVVGGSVGLAEGYLALVETYLAQEPAAFHVDLLAAHYRHDAGLLGAALLAQGEKL

SEQ ID NO: 20 - DNA, 990 bp, Escherichia coli
atgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagagttcgatccgatcctgctgcgccctgttgacgatctggaattgactgtccgctctgctaactgccttaaagcagaagctatccactatatcggtgatctggtacagcgtaccgaggttgagctccttaaaacgcctaaccttggtaaaaaatctcttactgagattaaagacgtgctggcttcccgtggactgtctctgggcatgcgcctggaaaactggccaccggcaagcatcgctgacgagtaa

SEQ ID NO: 21 - protein, 329 aa, Escherichia coli [sequence omitted]

SEQ ID NO: 22 - DNA, 4029 bp, Escherichia coli
atggtttactcctataccgagaaaaaacgtattcgtaaggattttggtaaacgtccacaagttctggatgtaccttatctcctttctatccagcttgactcgtttcagaaatttatcgagcaagatcctgaagggcagtatggtctggaagctgctttccgttccgtattcccgattcagagctacagcggtaattccgagctgcaatacgtcagctaccgccttggcgaaccggtgtttgacgtccaggaatgtcaaatccgtggcgtgacctattccgcaccgctgcgcgttaaactgcgtctggtgatctatgagcgcgaagcgccggaaggcaccgtaaaagacattaaagaacaagaagtctacatgggcgaaattccgctcatgacagacaacggtacctttgttatcaacggtactgagcgtgttatcgtttcccagctgcaccgtagtccgggcgtcttctttgactccgacaaaggtaaaacccactcttcgggtaaagtgctgtataacgcgcgtatcatcccttaccgtggttcctggctggacttcgaattcgatccgaaggacaacctgttcgtacgtatcgaccgtcgccgtaaactgcctgcgaccatcattctgcgcgccctgaactacaccacagagcagatcctcgacctgttctttgaaaaagttatctttgaaatccgtgataacaagctgcagatggaactggtgccggaacgcctgcgtggtgaaaccgcatcttttgacatcgaagctaacggtaaagtgtacgtagaaaaaggccgccgtatcactgcgcgccacattcgccagctggaaaaagacgacgtcaaactgatcgaagtcccggttgagtacatcgcaggtaaagtggttgctaaagactatattgatgagtctaccggcgagctgatctgcgcagcgaacatggagctgagcctggatctgctggctaagctgagccagtctggtcacaagcgtatcgaaacgctgttcaccaacgatctggatcacggcccatatatctctgaaaccttacgtgtcgacccaactaacgaccgtctgagcgcactggtagaaatctaccgcatgatgcgccctggcgagccgccgactcgtgaagcagctgaaagcctgttcgagaacctgttcttctccgaagaccgttatgacttgtctgcggttggtcgtatgaagttcaaccgttctctgctgcgcgaagaaatcgaaggttccggtatcctgagcaaagacgacatcattgatgttatgaaaaagctcatcgatatccgtaacggtaaaggcgaagtcgatgatatcgaccacctcggcaaccgtcgtatccgttccgttggcgaaatggcggaaaaccagttccgcgttggcctggtacgtgtagagcgtgcggtgaaagagcgtctgtctctgggcgatctggataccctgatgccacaggatatgatcaacgccaagccgatttccgcagcagtgaaagagttcttcggttccagccagctgtctcagtttatggaccagaacaacccgctgtctgagattacgcacaaacgtcgtatctccgcactcggcccaggcggtctgacccgtgaacgtgcaggcttcgaagttcgagacgtacacccgactcactacggtcgcgtatgtccaatcgaaacccctgaaggtccgaacatcggtctgatcaactctctgtccgtgtacgcacagactaacgaatacggcttccttgagactccgtatcgtaaagtgaccgacggtgttgtaactgacgaaattcactacctgtctgctatcgaagaaggcaactacgttatcgcccaggcgaactccaacttggatgaagaaggccacttcgtagaagacctggtaacttgccgtagcaaaggcgaatccagcttgttcagccgcgaccaggttgactacatggacgtatccacccagcaggtggtatccgtcggtgcgtccctgatcccgttcctggaacacgatgacgccaaccgtgcattgatgggtgcgaacatgcaacgtcaggccgttccgactctgcgcgctgataagccgctggttggtactggtatggaacgtgctgttgccgttgactccggtgtaactgcggtagctaaacgtggtggtgtcgttcagtacgtggatgcttcccgtatcgttatcaaagttaacgaagacgagatgtatccgggtgaagcaggtatcgacatctacaacctgaccaaatacacccgttctaaccagaacacctgtatcaaccagatgccgtgtgtgtctctgggtgaaccggttgaacgtggcgacgtgctggcagacggtccgtccaccgacctcggtgaactggcgcttggtcagaacatgcgcgtagcgttcatgccgtggaatggttacaacttcgaagactccatcctcgtatccgagcgtgttgttcaggaagaccgtttcaccaccatccacattcaggaactggcgtgtgtgtcccgtgacaccaagctgggtccggaagagatcaccgctgacatcccgaacgtgggtgaagctgcgctctccaaactggatgaatccggtatcgtttacattggtgcggaagtgaccggtggcgacattctggttggtaaggtaacgccgaaaggtgaaactcagctgaccccagaagaaaaactgctgcgtgcgatcttcggtgagaaagcctctgacgttaaagactcttctctgcgcgtaccaaacggtgtatccggtacggttatcgacgttcaggtctttactcgcgatggcgtagaaaaagacaaacgtgcgctggaaatcgaagaaatgcagctcaaacaggcgaagaaagacctgtctgaagaactgcagatcctcgaagcgggtctgttcagccgtatccgtgctgtgctggtagccggtggcgttgaagctgagaagctcgacaaactgccgcgcgatcgctggctggagctgggcctgacagacgaagagaaacaaaatcagctggaacagctggctgagcagtatgacgaactgaaacacgagttcgagaagaaactcgaagcgaaacgccgcaaaatcacccagggcgacgatctggcaccgggcgtgctgaagattgttaaggtatatctggcggttaaacgccgtatccagcctggtgacaagatggcaggtcgtcacggtaacaagggtgtaatttctaagatcaacccgatcgaagatatgccttacgatgaaaacggtacgccggtagacatcgtactgaacccgctgggcgtaccgtctcgtatgaacatcggtcagatcctcgaaacccacctgggtatggctgcgaaaggtatcggcgacaagatcaacgccatgctgaaacagcagcaagaagtcgcgaaactgcgcgaattcatccagcgtgcgtacgatctgggcgctgacgttcgtcagaaagttgacctgagtaccttcagcgatgaagaagttatgcgtctggctgaaaacctgcgcaaaggtatgccaatcgcaacgccggtgttcgacggtgcgaaagaagcagaaattaaagagctgctgaaacttggcgacctgccgacttccggtcagatccgcctgtacgatggtcgcactggtgaacagttcgagcgtccggtaaccgttggttacatgtacatgctgaaactgaaccacctggtcgacgacaagatgcacgcgcgttccaccggttcttacagcctggttactcagcagccgctgggtggtaaggcacagttcggtggtcagcgtttcggggagatggaagtgtgggcgctggaagcatacggcgcagcatacaccctgcaggaaatgctcaccgttaagtctgatgacgtgaacggtcgtaccaagatgtataaaaacatcgtggacggcaaccatcagatggagccgggcatgccagaatccttcaacgtattgttgaaagagattcgttcgctgggtatcaacatcgaactggaagacgagtaa

SEQ ID NO: 23 - protein, 1342 aa, Escherichia coli [sequence omitted]

SEQ ID NO: 24 - DNA, 4224 bp, Escherichia coli
gtgaaagatttattaaagtttctgaaagcgcagactaaaaccgaagagtttgatgcgatcaaaattgctctggcttcgccagacatgatccgttcatggtctttcggtgaagttaaaaagccggaaaccatcaactaccgtacgttcaaaccagaacgtgacggccttttctgcgcccgtatctttgggccggtaaaagattacgagtgcctgtgcggtaagtacaagcgcctgaaacaccgtggcgtcatctgtgagaagtgcggcgttgaagtgacccagactaaagtacgccgtgagcgtatgggccacatcgaactggcttccccgactgcgcacatctggttcctgaaatcgctgccgtcccgtatcggtctgctgctcgatatgccgctgcgcgatatcgaacgcgtactgtactttgaatcctatgtggttatcgaaggcggtatgaccaacctggaacgtcagcagatcctgactgaagagcagtatctggacgcgctggaagagttcggtgacgaattcgacgcgaagatgggggcggaagcaatccaggctctgctgaagagcatggatctggagcaagagtgcgaacagctgcgtgaagagctgaacgaaaccaactccgaaaccaagcgtaaaaagctgaccaagcgtatcaaactgctggaagcgttcgttcagtctggtaacaaaccagagtggatgatcctgaccgttctgccggtactgccgccagatctgcgtccgctggttccgctggatggtggtcgtttcgcgacttctgacctgaacgatctgtatcgtcgcgtcattaaccgtaacaaccgtctgaaacgtctgctggatctggctgcgccggacatcatcgtacgtaacgaaaaacgtatgctgcaggaagcggtagacgccctgctggataacggtcgtcgcggtcgtgcgatcaccggttctaacaagcgtcctctgaaatctttggccgacatgatcaaaggtaaacagggtcgtttccgtcagaacctgctcggtaagcgtgttgactactccggtcgttctgtaatcaccgtaggtccatacctgcgtctgcatcagtgcggtctgccgaagaaaatggcactggagctgttcaaaccgttcatctacggcaagctggaactgcgtggtcttgctaccaccattaaagctgcgaagaaaatggttgagcgcgaagaagctgtcgtttgggatatcctggacgaagttatccgcgaacacccggtactgctgaaccgtgcaccgactctgcaccgtctgggtatccaggcatttgaaccggtactgatcgaaggtaaagctatccagctgcacccgctggtttgtgcggcatataacgccgacttcgatggtgaccagatggctgttcacgtaccgctgacgctggaagcccagctggaagcgcgtgcgctgatgatgtctaccaacaacatcctgtccccggcgaacggcgaaccaatcatcgttccgtctcaggacgttgtactgggtctgtactacatgacccgtgactgtgttaacgccaaaggcgaaggcatggtgctgactggcccgaaagaagcagaacgtctgtatcgctctggtctggcttctctgcatgcgcgcgttaaagtgcgtatcaccgagtatgaaaaagatgctaacggtgaattagtagcgaaaaccagcctgaaagacacgactgttggccgtgccattctgtggatgattgtaccgaaaggtctgccttactccatcgtcaaccaggcgctgggtaaaaaagcaatctccaaaatgctgaacacctgctaccgcattctcggtctgaaaccgaccgttatttttgcggaccagatcatgtacaccggcttcgcctatgcagcgcgttctggtgcatctgttggtatcgatgacatggtcatcccggagaagaaacacgaaatcatctccgaggcagaagcagaagttgctgaaattcaggagcagttccagtctggtctggtaactgcgggcgaacgctacaacaaagttatcgatatctgggctgcggcgaacgatcgtgtatccaaagcgatgatggataacctgcaaactgaaaccgtgattaaccgtgacggtcaggaagagaagcaggtttccttcaacagcatctacatgatggccgactccggtgcgcgtggttctgcggcacagattcgtcagcttgctggtatgcgtggtctgatggcgaagccggatggctccatcatcgaaacgccaatcaccgcgaacttccgtgaaggtctgaacgtactccagtacttcatctccacccacggtgctcgtaaaggtctggcggataccgcactgaaaactgcgaactccggttacctgactcgtcgtctggttgacgtggcgcaggacctggtggttaccgaagacgattgtggtacccatgaaggtatcatgatgactccggttatcgagggtggtgacgttaaagagccgctgcgcgatcgcgtactgggtcgtgtaactgctgaagacgttctgaagccgggtactgctgatatcctcgttccgcgcaacacgctgctgcacgaacagtggtgtgacctgctggaagagaactctgtcgacgcggttaaagtacgttctgttgtatcttgtgacaccgactttggtgtatgtgcgcactgctacggtcgtgacctggcgcgtggccacatcatcaacaagggtgaagcaatcggtgttatcgcggcacagtccatcggtgaaccgggtacacagctgaccatgcgtacgttccacatcggtggtgcggcatctcgtgcggctgctgaatccagcatccaagtgaaaaacaaaggtagcatcaagctcagcaacgtgaagtcggttgtgaactccagcggtaaactggttatcacttcccgtaatactgaactgaaactgatcgacgaattcggtcgtactaaagaaagctacaaagtaccttacggtgcggtactggcgaaaggcgatggcgaacaggttgctggcggcgaaaccgttgcaaactgggacccgcacaccatgccggttatcaccgaagtaagcggttttgtacgctttactgacatgatcgacggccagaccattacgcgtcagaccgacgaactgaccggtctgtcttcgctggtggttctggattccgcagaacgtaccgcaggtggtaaagatctgcgtccggcactgaaaatcgttgatgctcagggtaacgacgttctgatcccaggtaccgatatgccagcgcagtacttcctgccgggtaaagcgattgttcagctggaagatggcgtacagatcagctctggtgacaccctggcgcgtattccgcaggaatccggcggtaccaaggacatcaccggtggtctgccgcgcgttgcggacctgttcgaagcacgtcgtccgaaagagccggcaatcctggctgaaatcagcggtatcgtttccttcggtaaagaaaccaaaggtaaacgtcgtctggttatcaccccggtagacggtagcgatccgtacgaagagatgattccgaaatggcgtcagctcaacgtgttcgaaggtgaacgtgtagaacgtggtgacgtaatttccgacggtccggaagcgccgcacgacattctgcgtctgcgtggtgttcatgctgttactcgttacatcgttaacgaagtacaggacgtataccgtctgcagggcgttaagattaacgataaacacatcgaagttatcgttcgtcagatgctgcgtaaagctaccatcgttaacgcgggtagctccgacttcctggaaggcgaacaggttgaatactctcgcgtcaagatcgcaaaccgcgaactggaagcgaacggcaaagtgggtgcaacttactcccgcgatctgctgggtatcaccaaagcgtctctggcaaccgagtccttcatctccgcggcatcgttccaggagaccactcgcgtgctgaccgaagcagccgttgcgggcaaacgcgacgaactgcgcggcctgaaagagaacgttatcgtgggtcgtctgatcccggcaggtaccggttacgcgtaccaccaggatcgtatgcgtcgccgtgctgcgggtgaagctccggctgcaccgcaggtgactgcagaagacgcatctgccagcctggcagaactgctgaacgcaggtctgggcggttctgataacgagtaa

SEQ ID NO: 25 - protein, 1407 aa, Escherichia coli [sequence omitted]

SEQ ID NO: 26 - DNA, 2109 bp, Escherichia coli
ttgtatctgtttgaaagcctgaatcaactgattcaaacctacctgccggaagaccaaatcaagcgtctgcggcaggcgtatctcgttgcacgtgatgctcacgaggggcaaacacgttcaagcggtgaaccctatatcacgcacccggtagcggttgcctgcattctggccgagatgaaactcgactatgaaacgctgatggcggcgctgctgcatgacgtgattgaagatactcccgccacctaccaggatatggaacagctttttggtaaaagcgtcgccgagctggtagagggggtgtcgaaacttgataaactcaagttccgcgataagaaagaggcgcaggccgaaaactttcgcaagatgattatggcgatggtgcaggatatccgcgtcatcctcatcaaacttgccgaccgtacccacaacatgcgcacgctgggctcacttcgcccggacaaacgtcgccgcatcgcccgtgaaactctcgaaatttatagcccgctggcgcaccgtttaggtatccaccacattaaaaccgaactcgaagagctgggttttgaggcgctgtatcccaaccgttatcgcgtaatcaaagaagtggtgaaagccgcgcgcggcaaccgtaaagagatgatccagaagattctttctgaaatcgaagggcgtttgcaggaagcgggaataccgtgccgcgtcagtggtcgcgagaagcatctttattcgatttactgcaaaatggtgctcaaagagcagcgttttcactcgatcatggacatctacgctttccgcgtgatcgtcaatgattctgacacctgttatcgcgtgctgggccagatgcacagcctgtacaagccgcgtccgggccgcgtgaaagactatatcgccattccaaaagcgaacggctatcagtctttgcacacctcgatgatcggcccgcacggtgtgccggttgaggtccagatccgtaccgaagatatggaccagatggcggagatgggtgttgccgcgcactgggcttataaagagcacggcgaaaccagtactaccgcacaaatccgcgcccagcgctggatgcaaagcctgctggagctgcaacagagcgccggtagttcgtttgaatttatcgagagcgttaaatccgatctcttcccggatgagatttacgttttcacaccggaagggcgcattgtcgagctgcctgccggtgcaacgcccgtcgacttcgcttatgcagtgcataccgatatcggtcatgcctgcgtgggcgcacgcgttgaccgccagccttacccgctgtcgcagccgcttaccagcggtcaaaccgttgaaatcattaccgctccgggcgctcgcccgaatgccgcttggctgaactttgtcgttagctcgaaagcgcgcgccaaaattcgtcagttgctgaaaaacctcaagcgtgatgattctgtaagcctgggccgtcgtctgctcaaccatgctttgggtggtagccgtaagctgaatgaaatcccgcaggaaaatattcagcgcgagctggatcgcatgaagctggcaacgcttgacgatctgctggcagaaatcggacttggtaacgcaatgagcgtggtggtcgcgaaaaatctgcaacatggggacgcctccattccaccggcaacccaaagccacggacatctgcccattaaaggtgccgatggcgtgctgatcacctttgcgaaatgctgccgccctattcctggcgacccgattatcgcccacgtcagccccggtaaaggtctggtgatccaccatgaatcctgccgtaatatccgtggctaccagaaagagccagagaagtttatggctgtggaatgggataaagagacggcgcaggagttcatcaccgaaatcaaggtggagatgttcaatcatcagggtgcgctggcaaacctgacggcggcaattaacaccacgacttcgaatattcaaagtttgaatacggaagagaaagatggtcgcgtctacagcgcctttattcgtctgaccgctcgtgaccgtgtgcatctggcgaatatcatgcgcaaaatccgcgtgatgccagacgtgattaaagtcacccgaaaccgaaattaa

SEQ ID NO: 27 - protein, 702 aa, Escherichia coli
MYLFESLNQLIQTYLPEDQIKRLRQAYLVARDAHEGQTRSSGEPYITHPVAVACILAEMKLDYETLMAALLHDVIEDTPATYQDMEQLFGKSVAELVEGVSKLDKLKFRDKKEAQAENFRKMIMAMVQDIRVILIKLADRTHNMRTLGSLRPDKRRRIARETLEIYSPLAHRLGIHHIKTELEELGFEALYPNRYRVIKEVVKAARGNRKEMIQKILSEIEGRLQEAGIPCRVSGREKHLYSIYCKMVLKEQRFHSIMDIYAFRVIVNDSDTCYRVLGQMHSLYKPRPGRVKDYIAIPKANGYQSLHTSMIGPHGVPVEVQIRTEDMDQMAEMGVAAHWAYKEHGETSTTAQIRAQRWMQSLLELQQSAGSSFEFIESVKSDLFPDEIYVFTPEGRIVELPAGATPVDFAYAVHTDIGHACVGARVDRQPYPLSQPLTSGQTVEIITAPGARPNAAWLNFVVSSKARAKIRQLLKNLKRDDSVSLGRRLLNHALGGSRKLNEIPQENIQRELDRMKLATLDDLLAEIGLGNAMSVVVAKNLQHGDASIPPATQSHGHLPIKGADGVLITFAKCCRPIPGDPIIAHVSPGKGLVIHHESCRNIRGYQKEPEKFMAVEWDKETAQEFITEIKVEMFNHQGALANLTAAINTTTSNIQSLNTEEKDGRVYSAFIRLTARDRVHLANIMRKIRVMPDVIKVTRNRN

SEQ ID NO: 28 - DNA, 546 bp, Escherichia coli
atgtctgaagctcctaaaaagcgctggtacgtcgttcaggcgttttccggttttgaaggccgcgtagcaacgtcgctgcgtgagcatatcaaattacacaacatggaagatttgtttggtgaagtcatggtaccaaccgaagaagtggttgaaatccgtggcggtcagcgtcgcaaaagcgaacgtaaattcttccctggctacgtcctcgttcagatggtgatgaacgacgcgagctggcacctggtgcgcagcgtaccgcgtgtgatgggcttcatcggcggtacttccgatcgtcctgcgccaatcagcgataaagaagtcgatgcgattatgaaccgcctgcagcaggttggtgataagccgcgtccgaaaacgctgtttgaaccgggtgaaatggtccgtgttaatgatggtccgttcgctgacttcaacggtgttgttgaagaagtggattacgagaaatctcgtctgaaagtgtctgtttctatcttcggtcgtgcgaccccggtagagctggacttcagccaggttgaaaaagcctaa

SEQ ID NO: 29 - protein, 181 aa, Escherichia coli
MSEAPKKRWYVVQAFSGFEGRVATSLREHIKLHNMEDLFGEVMVPTEEVVEIRGGQRRKSERKFFPGYVLVQMVMNDASWHLVRSVPRVMGFIGGTSDRPAPISDKEVDAIMNRLQQVGDKPRPKTLFEPGEMVRVNDGPFADFNGVVEEVDYEKSRLKVSVSIFGRATPVELDFSQVEKA

SEQ ID NO: 30 - DNA, 3120 bp, Escherichia coli
atgaaacgacatctgaatacctgctacaggctggtatggaatcacatgacgggcgctttcgtggttgcctccgaactggcccgcgcacggggtaaacgtggcggtgtggcggttgcactgtctcttgccgcagtcacgtcactcccggtgctggctgctgacatcgttgtgcacccgggagaaaccgtgaacggcggaacactggcaaatcatgacaaccagattgtcttcggtacgaccaacggaatgaccatcagtaccgggctggagtatgggccggataacgaggccaataccggcgggcaatgggtacaggatggcggaacagccaacaaaacgactgtcaccagtggtggtcttcagagagtgaaccccggtggaagtgtctcagacacggttatcagtgccggaggcggacagagccttcagggacgggctgtgaacaccacgctgaatggtggcgaacagtggatgcatgagggggcgatagccacaggaaccgtcattaatgataagggctggcaggtcgtcaagcccggtacagtggcaacggataccgttgttaataccggggcggaagggggaccggatgcagaaaacggtgataccgggcagtttgttcgcggggatgccgtacgcacaaccatcaataaaaacggtcgccagattgtgagagctgaaggaacggcaaataccactgtggtttatgccggcggcgaccagactgtacatggtcacgcactggataccacgctgaatgggggataccagtatgtgcacaacggcggtacagcgtctgacactgttgtgaacagtgacggctggcagattgtcaaaaacgggggtgtggccgggaataccaccgttaatcagaagggcagactgcaggtggacgccggtggtacagccacgaatgtcaccctgaagcagggcggcgcactggttaccagtacggctgcaaccgttaccggcataaaccgcctgggagcattctctgttgtggagggtaaagctgataatgtcgtactggaaaatggcggacgcctggatgtgctgaccggacacacagccactaatacccgcgtggatgatggcggaacgctggatgtccgcaacggtggcaccgccaccaccgtatccatgggaaatggcggtgtactgctggccgattccggtgccgctgtcagtggtacccggagcgacggaaaggcattcagtatcggaggcggtcaggcggatgccctgatgctggaaaaaggcagttcattcacgctgaacgccggtgatacggccacggataccacggtaaatggcggactgttcaccgccaggggcggcacactggcgggcaccaccacgctgaataacggcgccatacttaccctttccgggaagacggtgaacaacgataccctgaccatccgtgaaggcgatgcactcctgcagggaggctctctcaccggtaacggcagcgtggaaaaatcaggaagtggcacactcactgtcagcaacaccacactcacccagaaagccgtcaacctgaatgaaggcacgctgacgctgaacgacagtaccgtcaccacggatgtcattgctcagcgcggtacagccctgaagctgaccggcagcactgtgctgaacggtgccattgaccccacgaatgtcactctcgcctccggtgccacctggaatatccccgataacgccacggtgcagtcggtggtggatgacctcagccatgccggacagattcatttcacctccacccgcacagggaagttcgtaccggcaaccctgaaagtgaaaaacctgaacggacagaatggcaccatcagcctgcgtgtacgcccggatatggcacagaacaatgctgacagactggtcattgacggcggcagggcaaccggaaaaaccatcctgaacctggtgaacgccggcaacagtgcgtcggggctggcgaccagcggtaagggtattcaggtggtggaagccattaacggtgccaccacggaggaaggggcctttgtccaggggaacaggctgcaggccggtgcctttaactactccctcaaccgggacagtgatgagagctggtatctgcgcagtgaaaatgcttatcgtgcagaagtccccctgtatgcctccatgctgacacaggcaatggactatgaccggattgtggcaggctcccgcagccatcagaccggtgtaaatggtgaaaacaacagcgtccgtctcagcattcagggcggtcatctcggtcacgataacaatggcggtattgcccgtggggccacgccggaaagcagcggcagctatggattcgtccgtctggagggtgacctgatgagaacagaggttgccggtatgtctgtgaccgcgggggtatatggtgctgctggccattcttccgttgatgttaaggatgatgacggctcccgtgccggcacggtccgggatgatgccggcagcctgggcggatacctgaatctggtacacacgtcctccggcctgtgggctgacattgtggcacagggaacccgccacagcatgaaagcgtcatcggacaataacgacttccgcgcccggggctggggctggctgggctcactggaaaccggtctgcccttcagtatcactgacaacctgatgctggagccacaactgcagtatacctggcagggactttccctggatgacggtaaggacaacgccggttatgtgaagttcgggcatggcagtgcacaacatgtgcgtgccggtttccgtctgggcagccacaacgatatgacctttggcgaaggcacctcatcccgtgcccccctgcgtgacagtgcaaaacacagtgtgagtgaattaccggtgaactggtgggtacagccttctgttatccgcaccttcagctcccggggagatatgcgtgtggggacttccactgcaggcagcgggatgacgttctctccctcacagaatggcacatcactggacctgcaggccggactggaagcccgtgtccgggaaaatatcaccctgggcgttcaggccggttatgcccacagcgtcagcggcagcagcgctgaagggtataacggtcaggccacactgaatgtgaccttctga

SEQ ID NO: 31 - protein, 1039 aa, Escherichia coli
MKRHLNTCYRLVWNHMTGAFVVASELARARGKRGGVAVALSLAAVTSLPVLAADIVVHPGETVNGGTLANHDNQIVFGTTNGMTISTGLEYGPDNEANTGGQWVQDGGTANKTTVTSGGLQRVNPGGSVSDTVISAGGGQSLQGRAVNTTLNGGEQWMHEGAIATGTVINDKGWQVVKPGTVATDTVVNTGAEGGPDAENGDTGQFVRGDAVRTTINKNGRQIVRAEGTANTTVVYAGGDQTVHGHALDTTLNGGYQYVHNGGTASDTVVNSDGWQIVKNGGVAGNTTVNQKGRLQVDAGGTATNVTLKQGGALVTSTAATVTGINRLGAFSVVEGKADNVVLENGGRLDVLTGHTATNTRVDDGGTLDVRNGGTATTVSMGNGGVLLADSGAAVSGTRSDGKAFSIGGGQADALMLEKGSSFTLNAGDTATDTTVNGGLFTARGGTLAGTTTLNNGAILTLSGKTVNNDTLTIREGDALLQGGSLTGNGSVEKSGSGTLTVSNTTLTQKAVNLNEGTLTLNDSTVTTDVIAQRGTALKLTGSTVLNGAIDPTNVTLASGATWNIPDNATVQSVVDDLSHAGQIHFTSTRTGKFVPATLKVKNLNGQNGTISLRVRPDMAQNNADRLVIDGGRATGKTILNLVNAGNSASGLATSGKGIQVVEAINGATTEEGAFVQGNRLQAGAFNYSLNRDSDESWYLRSENAYRAEVPLYASMLTQAMDYDRIVAGSRSHQTGVNGENNSVRLSIQGGHLGHDNNGGIARGATPESSGSYGFVRLEGDLMRTEVAGMSVTAGVYGAAGHSSVDVKDDDGSRAGTVRDDAGSLGGYLNLVHTSSGLWADIVAQGTRHSMKASSDNNDFRARGWGWLGSLETGLPFSITDNLMLEPQLQYTWQGLSLDDGKDNAGYVKFGHGSAQHVRAGFRLGSHNDMTFGEGTSSRAPLRDSAKHSVSELPVNWWVQPSVIRTFSSRGDMRVGTSTAGSGMTFSPSQNGTSLDLQAGLEARVRENITLGVQAGYAHSVSGSSAEGYNGQATLNVTF

SEQ ID NO: 32 - DNA, 2355 bp, Escherichia coli
atgaatcctgagcgttctgaacgcattgaaatccccgtattgccgctgcgcgatgtggtggtttatccgcacatggtcatccccttatttgtcgggcgggaaaaatctatccgttgtctggaagcggcgatggaccatgataaaaaaattatgctggtcgcgcagaaagaagcttcaacggatgagccgggtgtaaacgatcttttcaccgtcgggaccgtggcctctatattgcagatgctgaaactgcctgacggcaccgtcaaagtgctggtcgaggggttacagcgcgcgcgtatttctgcgctctctgacaatggcgaacacttttctgcgaaggcggagtatctggagtcgccgaccattgatgagcgggaacaggaagtgctggtgcgtactgcaatcagccagttcgaaggctacatcaagctgaacaaaaaaatcccaccagaagtgctgacgtcgctgaatagcatcgacgatccggcgcgtctggcggataccattgctgcacatatgccgctgaaactggctgacaaacagtctgttctggagatgtccgacgttaacgaacgtctggaatatctgatggcaatgatggaatcggaaatcgatctgctgcaggttgagaaacgcattcgcaaccgcgttaaaaagcagatggagaaatcccagcgtgagtactatctgaacgagcaaatgaaagctattcagaaagaactcggtgaaatggacgacgcgccggacgaaaacgaagccctgaagcgcaaaatcgacgcggcgaagatgccgaaagaggcaaaagagaaagcggaagcagagttgcagaagctgaaaatgatgtctccgatgtcggcagaagcgaccgtagtgcgtggttatatcgactggatggtacaggtgccgtggaatgcgcgtagcaaggtcaaaaaagacctgcgtcaggcgcaggaaatccttgataccgaccattatggtctggagcgcgtgaaagatcgaatccttgagtatcttgcggttcaaagccgtgtcaacaaaatcaagggaccgatcctctgcctggtagggccgccgggggtaggtaaaacctctcttggtcagtccattgccaaagccaccgggcgtaaatatgtccgtatggcgctgggcggcgtgcgtgatgaagcggaaatccgtggtcaccgccgtacttacatcggttctatgccgggtaaactgatccagaaaatggcgaaagtgggcgtgaaaaacccgctgttcctgctcgatgagatcgacaaaatgtcttctgacatgcgtggcgatccggcctctgcactgcttgaagtgctggatccagagcagaacgtagcgttcagcgaccactacctggaagtggattacgatctcagcgacgtgatgtttgtcgcgacgtcgaactccatgaacattccggcaccgctgctggatcgtatggaagtgattcgcctctccggttataccgaagatgaaaaactgaacatcgccaaacgtcacctgctgccgaagcagattgaacgtaatgcactgaaaaaaggtgagctgaccgtcgacgatagcgccattatcggcattattcgttactacacccgtgaggcgggcgtgcgtggtctggagcgtgaaatctccaaactgtgtcgcaaagcggttaagcagttactgctcgataagtcattaaaacatatcgaaattaacggcgataacctgcatgactatctcggtgttcagcgtttcgactatggtcgcgcggataacgaaaaccgtgtcggtcaggtaaccggtctggcgtggacggaagtgggcggtgacttgctgaccattgaaaccgcatgtgttccgggtaaaggcaaactgacctataccggttcgctcggcgaagtgatgcaggagtccattcaggcggcgttaacggtggttcgtgcgcgtgcggaaaaactggggatcaaccctgatttttacgaaaaacgtgacatccacgtccacgtaccggaaggtgcgacgccgaaagatggtccgagtgccggtattgctatgtgcaccgcgctggtttcttgcctgaccggtaacccggttcgtgccgatgtggcaatgaccggtgagatcactctgcgtggtcaggtactgccgatcggtggtttgaaagaaaaactcctggcagcgcatcgcggcgggattaaaacagtgctaattccgttcgaaaataaacgcgatctggaagagattcctgacaacgtaattgccgatctggacattcatcctgtgaagcgcattgaggaagttctgactctggcgctgcaaaatgaaccgtctggtatgcaggttgtgactgcaaaatag

SEQ ID NO: 33 - protein, 784 aa, Escherichia coli
MNPERSERIEIPVLPLRDVVVYPHMVIPLFVGREKSIRCLEAAMDHDKKIMLVAQKEASTDEPGVNDLFTVGTVASILQMLKLPDGTVKVLVEGLQRARISALSDNGEHFSAKAEYLESPTIDEREQEVLVRTAISQFEGYIKLNKKIPPEVLTSLNSIDDPARLADTIAAHMPLKLADKQSVLEMSDVNERLEYLMAMMESEIDLLQVEKRIRNRVKKQMEKSQREYYLNEQMKAIQKELGEMDDAPDENEALKRKIDAAKMPKEAKEKAEAELQKLKMMSPMSAEATVVRGYIDWMVQVPWNARSKVKKDLRQAQEILDTDHYGLERVKDRILEYLAVQSRVNKIKGPILCLVGPPGVGKTSLGQSIAKATGRKYVRMALGGVRDEAEIRGHRRTYIGSMPGKLIQKMAKVGVKNPLFLLDEIDKMSSDMRGDPASALLEVLDPEQNVAFSDHYLEVDYDLSDVMFVATSNSMNIPAPLLDRMEVIRLSGYTEDEKLNIAKRHLLPKQIERNALKKGELTVDDSAIIGIIRYYTREAGVRGLEREISKLCRKAVKQLLLDKSLKHIEINGDNLHDYLGVQRFDYGRADNENRVGQVTGLAWTEVGGDLLTIETACVPGKGKLTYTGSLGEVMQESIQAALTVVRARAEKLGINPDFYEKRDIHVHVPEGATPKDGPSAGIAMCTALVSCLTGNPVRADVAMTGEITLRGQVLPIGGLKEKLLAAHRGGIKTVLIPFENKRDLEEIPDNVIADLDIHPVKRIEEVLTLALQNEPSGMQVVTAK

SEQ ID NO: 34 - DNA, 336 bp, Escherichia coli
atgagctatgaggttctgctgcttgggttactagttggcgtggcgaattattgcttccgctatttgccgctgcgcctgcgtgtgggtaatgcccgcccaaccaaacgtggcgcggtaggtattttgctcgacaccattggcatcgcctcgatatgcgctctgctggttgtctctaccgcaccagaagtgatgcacgatacacgccgtttcgtgcccacgctggtcggcttcgcggtactgggtgccagtttctataaaacacgcagcattatcatcccaacactgcttagtgcgctggcctatgggctcgcctggaaagtgatggcgattatataa

SEQ ID NO: 35 - protein, 111 aa, Escherichia coli
MSYEVLLLGLLVGVANYCFRYLPLRLRVGNARPTKRGAVGILLDTIGIASICALLVVSTAPEVMHDTRRFVPTLVGFAVLGASFYKTRSIIIPTLLSALAYGLAWKVMAII

SEQ ID NO: 36 - DNA, 1674 bp, Escherichia coli
atgactgaatcttttgctcaactctttgaagagtccttaaaagaaatcgaaacccgcccgggttctatcgttcgtggcgttgttgttgctatcgacaaagacgtagtactggttgacgctggtctgaaatctgagtccgccatcccggctgagcagttcaaaaacgcccagggcgagctggaaatccaggtaggtgacgaagttgacgttgctctggacgcagtagaagacggcttcggtgaaactctgctgtcccgtgagaaagctaaacgtcacgaagcctggatcacgctggaaaaagcttacgaagatgctgaaactgttaccggtgttatcaacggcaaagttaagggcggcttcactgttgagctgaacggtattcgtgcgttcctgccaggttctctggtagacgttcgtccggtgcgtgacactctgcacctggaaggcaaagagcttgaatttaaagtaatcaagctggatcagaagcgcaacaacgttgttgtttctcgtcgtgccgttatcgaatccgaaaacagcgcagagcgcgatcagctgctggaaaacctgcaggaaggcatggaagttaaaggtatcgttaagaacctcactgactacggtgcattcgttgatctgggcggcgttgacggcctgctgcacatcactgacatggcctggaaacgcgttaagcatccgagcgaaatcgtcaacgtgggcgacgaaatcactgttaaagtgctgaagttcgaccgcgaacgtacccgtgtatccctgggcctgaaacagctgggcgaagatccgtgggtagctatcgctaaacgttatccggaaggtaccaaactgactggtcgcgtgaccaacctgaccgactacggctgcttcgttgaaatcgaagaaggcgttgaaggcctggtacacgtttccgaaatggactggaccaacaaaaacatccacccgtccaaagttgttaacgttggcgatgtagtggaagttatggttctggatatcgacgaagaacgtcgtcgtatctccctgggtctgaaacagtgcaaagctaacccgtggcagcagttcgcggaaacccacaacaagggcgaccgtgttgaaggtaaaatcaagtctatcactgacttcggtatcttcatcggcttggacggcggcatcgacggcctggttcacctgtctgacatctcctggaacgttgcaggcgaagaagcagttcgtgaatacaaaaaaggcgacgaaatcgctgcagttgttctgcaggttgacgcagaacgtgaacgtatctccctgggcgttaaacagctcgcagaagatccgttcaacaactgggttgctctgaacaagaaaggcgctatcgtaaccggtaaagtaactgcagttgacgctaaaggcgcaaccgtagaactggctgacggcgttgaaggttacctgcgtgcttctgaagcatcccgtgaccgcgttgaagacgctaccctggttctgagcgttggcgacgaagttgaagctaaattcaccggcgttgatcgtaaaaaccgcgcaatcagcctgtctgttcgtgcgaaagacgaagctgacgagaaagatgcaatcgcaactgttaacaaacaggaagatgcaaacttctccaacaacgcaatggctgaagctttcaaagcagctaaaggcgagtaa

SEQ ID NO: 37 - protein, 557 aa, Escherichia coli
MTESFAQLFEESLKEIETRPGSIVRGVVVAIDKDVVLVDAGLKSESAIPAEQFKNAQGELEIQVGDEVDVALDAVEDGFGETLLSREKAKRHEAWITLEKAYEDAETVTGVINGKVKGGFTVELNGIRAFLPGSLVDVRPVRDTLHLEGKELEFKVIKLDQKRNNVVVSRRAVIESENSAERDQLLENLQEGMEVKGIVKNLTDYGAFVDLGGVDGLLHITDMAWKRVKHPSEIVNVGDEITVKVLKFDRERTRVSLGLKQLGEDPWVAIAKRYPEGTKLTGRVTNLTDYGCFVEIEEGVEGLVHVSEMDWTNKNIHPSKVVNVGDVVEVMVLDIDEERRRISLGLKQCKANPWQQFAETHNKGDRVEGKIKSITDFGIFIGLDGGIDGLVHLSDISWNVAGEEAVREYKKGDEIAAVVLQVDAERERISLGVKQLAEDPFNNWVALNKKGAIVTGKVTAVDAKGATVELADGVEGYLRASEASRDRVEDATLVLSVGDEVEAKFTGVDRKNRAISLSVRAKDEADEKDAIATVNKQEDANFSNNAMAEAFKAAKGE

(19) United States
(12) Patent Application Publication
(10) Pub. No.: US 2019/0309309 A1
(43) Pub. Date: Oct. 10, 2019

(54) BACTERIAL CELLS WITH IMPROVED TOLERANCE TO POLYOLS

(71) Applicant: DANMARKS TEKNISKE UNIVERSITET, Kgs. Lyngby (DK)

(72) Inventors: Rebecca Lennen, Holte (DK); Alex Toftgaard Nielsen, Rungsted Kyst (DK); Markus Herrgård, Virum (DK); Morten Sommer, Hellerup (DK); Adam Feist, San Diego, CA (US); Elsayed Tharwat Tolba Mohamed, Helsingborg (SE)

(21) Appl. No.: 16/307,825

(22) PCT Filed: Jun. 7, 2017
(86) PCT No.: PCT/EP2017/063821

§ 371 Date: Dec. 6, 2018

Related U.S. Application Data
(60) Provisional application No. 62/346,804, filed on Jun. 7, 2016.

(30) Foreign Application Priority Data
Jun. 27, 2016 (EP) 16176365.1

(51) Int. Cl. C12N 15/52; C12P 7/18; C12N 9/10; C12N 9/08; C07K 14/245; C12N 9/52; C12N 9/22

(52) U.S. Cl. CPC C12N 15/52; C12P 7/18; C12N 9/1077; C12N 9/22; C07K 14/245; C12N 9/52; C12N 9/0065

(57) ABSTRACT

The present invention relates to bacterial cells genetically modified to improve their tolerance to certain commodity chemicals, such as diols and other polyols, and to methods of preparing and using such bacterial cells for production of polyols and other compounds.

### FIELD OF THE INVENTION

The present invention relates to bacterial cells genetically modified to improve their tolerance to certain commodity chemicals, such as diols and other polyols, and to methods of preparing and using such bacterial cells for production of polyols and other compounds.

### BACKGROUND OF THE INVENTION

Polyols such as diols are versatile water-miscible compounds used in diverse applications including use as polyester and polyurethane resin precursors, antifreezes, synthetic lubricants, plasticizers and polymer additives, intermediates in the production of pharmaceuticals and fragrances, and as food, cosmetic, and pharmaceutical ingredients (Werle et al., 2012; Köpnick et al., 2012). The predominant industrial use of diols is in the production of polyesters, as nearly all commercially produced polyesters are the product of esterification of dicarboxylic acids with diols (Werle et al., 2012). For example, the dominant use of 1,2-propanediol (45%) is in unsaturated polyester resins (Sullivan et al., 2012). Because of its safety to humans, it is also used in numerous food and cosmetic products, and as a lubricant, antifreeze, and aircraft deicer (Sullivan et al., 2012). Another diol, 2,3-butanediol, can be reduced to butadiene, a component of synthetic rubber, and the largest current usage of 2,3-butanediol is as a cross-linking agent for hard rubbers, as a precursor for insecticides, and as pharmaceutical intermediates (Grafje et al., 2012).

In the past, butadiene was primarily synthesized from butene obtained from cracked naptha. The recent increase in natural gas production via fracking, coupled with previously high oil prices, however, resulted in an increased price for C₄ and higher hydrocarbons which in turn resulted in a renewed interest in biological production of C₄ compounds such as 1,4-butanediol and 2,3-butanediol. Further, diols that contain stereocenters exist in different stereoisomers, and the use of stereoisomers can impart different physical properties to polymers. Utilization of a specific stereoisomer can also be useful for the purpose of introducing stereocenters into more complex compounds when they are used as intermediates. Biological production of diols can be particularly advantageous when compared to chemical synthesis, in that it can readily allow the production of pure stereoisomers or racemic mixtures of stereoisomers, depending on the enzymes employed. The production of diols in metabolically engineered microbial cells have been reviewed and described in several publications such as, e.g., Sabra et al. (2016), Clomburg et al. (2011), Jain et al. (2015), Li et al. (2015) and Xu et al. (2014).

For production of bulk chemicals from renewable plant-based carbon feedstocks, high product titers are essential in order to minimize capital equipment and downstream separations costs for product purification. At the high titers required for economical fermentation processes, however, most chemicals exhibit significant toxicity that reduce yields and productivities by negatively affecting microbial growth (Van Dien, 2013; Zingaro et al., 2013).

Escherichia coli being a suitable host for industrial applications, there has been some interest in developing E. coli strains with improved tolerance to chemicals of interest for production, such as, e.g., n-butanol, ethanol and isobutanol, or to stress conditions present during fermentation (see, e.g., Haft et al, 2014; Sandberg et al., 2014; Lennen and Herrgård, 2014; Tenaillon et al., 2012; Minty et al., 2011; Dragosits et al., 2013; Winkler et al., 2014; Wu et al., 2014; LaCroix et al., 2015; Jensen et al., 2015 and 2016; Doukyu et al., 2012; Shenhar et al., 2012; and Rath and Jawali, 2006).

Despite these and other advances in the art, there is still a need for bacterial cells with improved tolerance to chemicals of interest for bio-based production, such as diols and other polyols. It is an object of the invention to provide such bacterial cells.

### SUMMARY OF THE INVENTION

It has been found by the present inventors that certain genetic modifications unexpectedly improve the tolerance of bacterial cells, such as those of, e.g., the Escherichia genera, to certain chemical compounds, particularly aliphatic diols and other aliphatic polyols.

Accordingly, the invention provides bacterial cells with improved tolerance to at least one aliphatic polyol, as well as bacterial cells which are capable of producing an aliphatic polyol and have improved tolerance to the aliphatic polyol. Particularly contemplated are aliphatic diols, such as e.g., 2,3-butanediol; 1,2-propanediol; 1,4-butanediol; 1,3-propanediol; 1,2-butanediol; 1,5 pentanediol and/or 1,2-pentanediol.

The invention also relates to compositions comprising such bacterial cells and one or more aliphatic polyols, methods of preparing or screening for such bacterial cells, and methods of producing aliphatic polyols using such bacterial cells.

The invention also relates to methods of producing a diol or other polyol using bacterial cells, comprising supplementing the medium with methionine, wherein the concentration of methionine is from about 0.004 to about 0.2 g gDCW⁻¹ (gDCW=grams dry cell weight). These and other aspects and embodiments are described further below.

### DETAILED DISCLOSURE OF THE INVENTION

In this work, 2,3-butanediol and 1,2-propanediol were selected for performing adaptive laboratory evolutions. Based on the findings reported herein, various aspects of the invention provide for genetically modified bacterial host cells with a higher tolerance to one or more diols or other polyols. When transformed with a recombinant biosynthetic pathway for producing the polyol from a carbon source, the genetically modified bacterial host cells of the invention result in improved production of the polyol from carbon feedstock, since they maintain robust metabolic activity in the presence of higher concentrations of the polyol than the unmodified parent cells.

So, in one aspect, the bacterial cell comprises a biosynthetic, optionally recombinant, pathway for producing an aliphatic polyol and at least one genetic modification which reduces expression of an endogenous gene selected from the group consisting of metJ, iscR, yhjA, gtrS, ycdU, rzpD, sspA and rph, or a combination of any thereof, optionally wherein the cell further comprises a genetic modification which increases the expression of PyrE and/or a mutation in one or more of NanK, RpsA, RpoB, RpoC, SpoT, NusG, Flu, Lon, and YgaH.

In one aspect, the bacterial cell comprises a biosynthetic, optionally recombinant, pathway for producing an aliphatic polyol and at least one genetic modification which increases one or more of (a) the biosynthesis of methionine in the bacterial cell; (b) the growth of the bacterial cell during polyol-induced methionine starvation; (c) intracellular iron levels during polyol-induced growth inhibition; (d) biosynthesis of iron siderophores during polyol-induced growth inhibition; and (e) the biosynthesis of iron-sulfur clusters during polyol-induced growth inhibition.

In one embodiment of any aspect, the bacterial cell comprises at least one genetic modification which reduces expression of metJ and/or iscR. The bacterial cell may further comprise genetic modifications which reduce expression of relA and purT; or genetic modifications which reduce the expression of acrB, acrA, or both, optionally in combination with a genetic modification which increases the expression of PyrE, or a mutation in one or more of NanK, RpsA, RpoB, RpoC, SpoT, NusG, Flu, Lon and YgaH.

In one aspect, the bacterial cell comprises genetic modifications which reduce expression of metJ, relA and purT; metJ and acrB and/or acrA; iscR and relA; or fabR and ygfF, optionally in combination with a genetic modification which increases the expression of PyrE, and/or a mutation in one or more of NanK, RpsA, RpoB, RpoC, SpoT, NusG, Flu, Lon, and YgaH. Preferred examples of mutations are disclosed herein.

In one embodiment of any aspect, the genetic modification comprises a knock-down or knock-out of the endogenous gene. In a particular embodiment, the genetic modification is a knock-out.

Preferred, non-limiting polyols include diols, the genetic modification providing for an increased growth rate, a reduced lag time, or both, of the cell in at least one of, e.g., 2,3-butanediol and 1,2-propanediol, as compared to a control. The control may be, for example, the parent bacterial cell.

In one embodiment, the pathway is a recombinant pathway. For example, the bacterial cell may comprise a recombinant biosynthetic pathway for producing at least one of a propanediol, butanediol, pentanediol and a hexanediol.

The bacterial cell may be of any suitable genus or origin. Preferred, non-limiting genera include Escherichia, Enterobacter, Klebsiella, Lactobacillus, Lactococcus, Bacillus, Pseudomonas, Corynebacterium, Ralstonia, Paenibacillus, Clostridia and Citrobacter sp. Escherichia coli is particularly preferred.

In one aspect, there is provided a process for preparing a recombinant E. coli cell for producing an aliphatic polyol, comprising genetically modifying an E. coli cell to
(a) introduce a recombinant biosynthetic pathway for producing an aliphatic polyol; and knock-down or knock-out at least one endogenous gene selected from the group consisting of metJ, rzpD, yhjA, gtrS, ycdU, iscR, sspA and rph; or
(b) knock-down or knock-out a combination of endogenous genes selected from metJ, relA and purT; metJ and acrB and/or acrA; iscR and relA; and fabR and ygfF.

In one aspect, there is provided a process for improving the tolerance of a bacterial cell to an aliphatic diol, comprising genetically modifying the bacterial cell to knock-down or knock-out
(a) at least one endogenous gene selected from the group consisting of metJ, rzpD, yhjA, gtrS, ycdU, iscR, sspA and rph; or
(b) a combination of endogenous genes selected from metJ, relA and purT; metJ and acrB and/or acrA; iscR and relA; and fabR and ygfF,

optionally also introducing a genetic modification which increases the expression of PyrE, or a mutation in one or more of NanK, RpsA, RpoB, RpoC, SpoT, NusG, Flu, Lon, and YgaH.

In one aspect, there is provided a process for preparing a recombinant E. coli cell for producing an aliphatic polyol, comprising genetically modifying an E. coli cell to introduce a recombinant biosynthetic pathway for producing an aliphatic polyol, and
(a) knock-down or knock-out at least one endogenous gene selected from the group consisting of metJ, iscR, yhjA, gtrS, ycdU, rzpD, sspA and rph; or a combination of endogenous genes selected from metJ, relA and purT; metJ and acrB, acrA or both; iscR and relA; and fabR and ygfF; and
(b) optionally, upregulating and/or introducing one or more mutations in at least one protein selected from NanK (SEQ ID NO:19), RpsA (SEQ ID NO:37), RpoA (SEQ ID NO:21); RpoB (SEQ ID NO:23), RpoC (SEQ ID NO:25), SpoT (SEQ ID NO:27), NusG (SEQ ID NO:29, Flu (SEQ ID NO:31), Lon (SEQ ID NO:33), and YgaH (SEQ ID NO:35), optionally wherein the one or more mutations are selected from RpoC-L268K, RpoC-L268N, RpoC-L268Q, RpoC-L268R, RpoC-N309F, RpoC-N309S, RpoC-N309T, RpoC-N309W, RpoC-N309Y, RpoC-Y75A, RpoC-Y75C, RpoC-Y75S, RpoC-LTPVIE(822-827), RpoB-D549A, RpoB-D549G, RpoB-H447F, RpoB-H447S, RpoB-H447T, RpoB-H447W, RpoB-H447Y, RpoB-11112S, RpoB-I1112T, RpoB-V931A, RpoB-V931I, RpoB-V931L, NanK-T128S, Flu-L642E, Flu-L642N, Flu-L642Q, Lon-1716S, Lon-1716T, YgaH-V39A, YgaH-V39I, YgaH-V39L, NusG-F144A, NusG-F144I, NusG-F144L, NusG-F144M, NusG-F144V, RpoA-D305A, RpoA-D305G, RpoA-G279A, RpoA-G279F, RpoA-G279I, RpoA-G279L, RpoA-G279M, RpoA-G279V, RpsA-D310A, RpsA-D310F, RpsA-D310I, RpsA-D310L, RpsA-D310M, RpsA-D310V, RpsA-G21A, RpsA-G21F, RpsA-G21I, RpsA-G21L, RpsA-G21M, RpsA-G21V, SpoT-I213A, SpoT-I213F, SpoT-I213L, SpoT-I213M, and SpoT-I213V.

In one aspect, there is provided a process for improving the tolerance of a bacterial cell to an aliphatic polyol, comprising genetically modifying the bacterial cell to
(a) knock-down or knock-out at least one endogenous gene selected from the group consisting of metJ, iscR, yhjA, gtrS, ycdU, rzpD, sspA and rph; or a combination of endogenous genes selected from metJ, relA and purT; metJ and acrB, acrA or both; iscR and relA; and fabR and ygfF;
(b) optionally introducing one or more mutations in one or more endogenous genes selected from NanK (SEQ ID NO:19), RpsA (SEQ ID NO:37), RpoA (SEQ ID NO:21); RpoB (SEQ ID NO:23), RpoC (SEQ ID NO:25), SpoT (SEQ ID NO:27), NusG (SEQ ID NO:29, Flu (SEQ ID NO:31), Lon (SEQ ID NO:33), and YgaH (SEQ ID NO:35) or the pyrE/rph intergenic region;
(c) preparing a population of the genetically modified bacterial cell; and
(d) selecting from the population in (c) any bacterial cell which has an improved tolerance to the aliphatic polyol.

In one aspect, there is provided a method for producing an aliphatic polyol, comprising culturing the bacterial cell of any aspect or embodiment herein in the presence of a carbon source, and, optionally, isolating the aliphatic polyol.

In one aspect, there is provided a composition comprising a propanediol or a butanediol at a concentration of at least 6% and a plurality of bacterial cells according to any aspect or embodiment herein. The bacterial cells may be, e.g., of the Escherichia genus, genetically modified to knock-down or knock-out at least one endogenous gene selected from the group consisting of metJ, rzpD, yhjA, gtrS, ycdU, iscR, sspA and rph; or a combination of endogenous genes selected from metJ, relA and purT; metJ and acrB and/or acrA; iscR and relA; and fabR and ygfF.

In one aspect, there is provided a method for producing an aliphatic diol, comprising (a) culturing a plurality of bacterial cells capable of producing the aliphatic diol in a medium comprising a carbon source, and (b) adding methionine to the medium, wherein the concentration of the added methionine is from about 0.004 g L gDCW⁻¹ to about 0.2 g L gDCW⁻¹, optionally wherein the bacterial cell is the bacterial cell of any preceding aspect or embodiment.

### Definitions

Unless otherwise indicated or contradicted by context, a “diol” as used herein is an aliphatic diol, and a “polyol” is an aliphatic polyol. An “aliphatic polyol” herein refers to an organic compound comprising an aliphatic carbon chain to which two or more hydroxyl (—OH) groups are attached, and includes linear aliphatic diols and other linear aliphatic polyols, as well as derivatives thereof. Aliphatic polyols suitable for production in bacteria typically comprise from 3 to 12 carbon atoms, preferably 3 to 10 carbon atoms, more preferably 3 to 8 carbon atoms, and, most preferably, 3 to 6 carbon atoms, and, optionally comprises one or more heteroatoms. Linear aliphatic polyols comprising 2, 3 or 4 hydroxyl groups are preferred and include, but are not limited to, 2,3-butanediol; 1,2-propanediol; 1,5 pentanediol; 1,2-pentanediol; 1,4-butanediol; 1,3-propanediol; 1,2-butanediol; 1,6-hexanediol; 1,8-octanediol; 1,10-decanediol and 1,12-dodecanediol. Particularly contemplated are propanediols, butanediols, pentanediols and hexanediols. Linear aliphatic diols such as, e.g., 2,3-butanediol; 1,2-propanediol; 1,5-pentanediol; 1,2-pentanediol; 1,6-hexanediol; 1,4-butanediol and 1,3-propanediol are most preferred.

As used herein, a “recombinant biosynthetic pathway” for a compound of interest refers to an enzymatic pathway resulting in the production of a compound of interest in a host cell, wherein at least one of the enzymes is expressed from a transgene, i.e., a gene added to the host cell genome by transformation. In some cases, the recombinant biosynthetic pathway also comprises a deletion of one or more native genes in the host cell. The compound of interest is typically a diol or other polyol, and may be the actual end product or a precursor or intermediate in the production of another end product.

The terms “tolerant” or “improved tolerance”, when used to describe a genetically modified bacterial cell of the invention or a strain derived therefrom, refers to a genetically modified bacterial cell or strain that shows a reduced lag time, an improved growth rate, or both, in the presence of a diol or other polyol than the parent bacterial cell or strain from which it is derived, typically at concentrations of at least 1% v/v, such as at least 1.5% v/v, such as at least 3% v/v, such as at least 5% v/v, such as at least 6% v/v, such as at least 7% v/v, such as at least 7.5% v/v, such as at least 8% v/v, such as at least 10% v/v. An improved growth rate is at least 5%, such as at least 10%, such as at least 20%, such as at least 50%, such as at least 75% higher than that of a control, typically the parent cell or strain. A reduced lag time is at least 10%, such as at least 20%, such as at least 50%, such as at least 75%, such as at least 90% shorter than that of a control, typically the parent cell or strain.

The term “gene” refers to a nucleic acid sequence that encodes a cellular function, such as a protein, optionally including regulatory sequences preceding (5′ non-coding sequences) and following (3′ non-coding sequences) the coding sequence. An “endogenous gene” refers to a native gene in its natural location in the genome of an organism. A “transgene” is a gene, native or heterologous, that has been introduced into the genome by a transformation procedure. Genes names are herein set forth in italicised text with a lower-case first letter (e.g., metJ) whereas protein names are set forth in normal text with a capital first letter (e.g., MetJ).

As used herein the term “coding sequence” refers to a DNA sequence that encodes a specific amino acid sequence.

The term “native”, when used to characterize a gene or a protein herein with respect to a host cell, refers to a gene or protein having the nucleic acid or amino acid sequence as found in the host cell.

The term “heterologous”, when used to characterize a gene or protein with respect to a host cell, refers to a gene or protein which has a nucleic acid or amino acid sequence not normally found in the host cell.

As used herein the term “transformation” refers to the transfer of a nucleic acid fragment, such as a gene, into a host cell. Host cells containing a gene introduced by transformation or a “transgene” are referred to as “transgenic” or “recombinant” or “transformed” cells.

As used herein, a “genetic modification” refers to the introduction a genetically inherited change in the host cell genome. Examples of changes include mutations in genes and regulatory sequences, coding and non-coding DNA sequences. “Mutations” include deletions, substitutions and insertions of one or more nucleotides or nucleic acid sequences in the genome. Other genetic modifications include the introduction of heterologous genes or coding DNA sequences by recombinant techniques.

The term “expression”, as used herein, refers to the process in which a gene is transcribed into mRNA, and may optionally include the subsequent translation of the mRNA into an amino acid sequence, i.e., a protein or polypeptide.

As used herein, “reduced expression” or “downregulation” of an endogenous gene in a host cell means that the levels of the mRNA, protein and/or protein activity encoded by the gene are significantly reduced in the host cell, typically by at least 25%, such as at least 50%, such as at least 75%, such as at least 90%, such as at least 95%, as compared to a control. Typically, when the reduced expression is obtained by a genetic modification in the host cell, the control is the unmodified host cell. Sometimes, e.g., in the case of gene knock-out, the reduction of native mRNA and functional protein encoded by the gene is higher, such as 99% or greater.

“Increased expression”, “upregulation”, “overexpressing” or the like, when used in the context of a protein or activity described herein, means increasing the protein level or activity within a bacterial cell. An up-regulation of an activity can occur through, e.g., increased activity of a protein, increased potency of a protein or increased expression of a protein. The protein with increased activity, potency or expression can be encoded by genes disclosed herein.

Genetic modifications resulting in a reduced expression of a target gene/protein can include, e.g., knock-down of the gene (e.g., a mutation in a promoter or other expression control sequence that results in decreased gene expression), a knock-out or disruption of the gene (e.g., a mutation or deletion of the gene that results in 99 percent or greater decrease in gene expression), a mutation or deletion in the coding sequence which results in the expression of non-functional protein, and/or the introduction of a nucleic acid sequence that reduces the expression of the target gene, e.g. a repressor that inhibits expression of the target or inhibitory nucleic acids (e.g. CRISPR etc.) that reduces the expression of the target gene.

Standard recombinant DNA and molecular cloning techniques used here are well known in the art and are described by Sambrook, J., Fritsch, E. F. and Maniatis, T. Molecular Cloning: A Laboratory Manual, 4^{th} ed.; Cold Spring Harbor Laboratory: Cold Spring Harbor, N.Y., 2012; and by Silhavy, T. J., Bennan, M. L. and Enquist, L. W. Experiments with Gene Fusions; Cold Spring Harbor Laboratory: Cold Spring Harbor, N.Y., 1984; and by Ausubel, F. M. et al., In Current Protocols in Molecular Biology, published by John Wiley & Sons (1995); and by Datsenko and Wanner, 2000; and by Baba et al., 2006; and by Thomason et al., 2007.

A “conservative” amino acid substitution in a protein is one that does not negatively influence protein activity. Typically, a conservative substitution can be made within groups of amino acids sharing physicochemical properties, such as, e.g., basic amino acids (arginine, lysine and histidine), acidic amino acids (glutamic acid and aspartic acid), polar amino acids (glutamine and asparagines), hydrophobic amino acids (leucine, isoleucine, valine and methionine), aromatic amino acids (phenylalanine, tryptophan and tyrosine), and small amino acids (glycine, alanine, serine, and threonine). Most commonly, substitutions can be made between Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, Asp/Gly. Other preferred substitutions are set out in Table 1 below.

TABLE 1
Examples of amino acid substitutions

| Original | | |
|---|---|---|
| amino acid | Examples of substitutions | Preferred substitution |
| Ala (A) | val; leu; ile | Val |
| Arg (R) | lys; gln; asn | Lys |
| Asn (N) | gln; his; asp, lys; arg | Gln |
| Asp (D) | glu; asn | Glu |
| Cys (C) | ser; ala | Ser |
| Gln (Q) | asn; glu | Asn |
| Glu (E) | asp; gln | Asp |
| Gly (G) | Ala | Ala |
| His (H) | asn; gln; lys; arg | Arg |
| Ile (I) | leu; val; met; ala; phe; norleucine | Leu |
| Leu (L) | norleucine; ile; val; met; ala; phe | Ile |
| Lys (K) | arg; gln; asn | Arg |
| Met (M) | leu; phe; ile | Leu |
| Phe (F) | leu; val; ile; ala; tyr | Tyr |
| Pro (P) | Ala | Ala |
| Ser (S) | thr | Thr |
| Thr (T) | Ser | Ser |
| Trp (W) | tyr; phe | Tyr |
| Tyr (Y) | trp; phe; thr; ser | Phe |
| Val (V) | ile; leu; met; phe; ala; norleucine | Leu |

### SPECIFIC EMBODIMENTS OF THE INVENTION

As described in the Examples, the growth rate of native K-12 MG1655 cells steadily decreased as a function of diol concentration, starting already at 0.5% or 1% v/v, with toxicity apparently depending on carbon chain length. Maximum concentrations for robust growth in 2,3-butanediol and 1,2-propanediol were 5% and 7.5%, respectively.

So, the invention provides bacterial cells with improved tolerance to diols and other polyols, as well as related processes and materials for producing and using such bacterial cells.

1) Genetic Modifications

The genetic modifications according to the invention include those resulting in reduced expression of genes, e.g., by gene knock-down or knock-out, herein referred to as “Group 1 modifications”; as well as silent mutations in coding or non-coding regions and non-silent (i.e., coding) mutations in coding regions, herein referred to as “Group 2 modifications”; and combinations thereof.

In a preferred embodiment, the one or more genetic modifications provide for an increased growth rate, a reduced lag time, or both, of the bacterial cell in at least one of 2,3-butanediol and 1,2-propanediol, e.g., at a concentration of at least 6% or at least 7% as compared to the wild-type bacterial cell.

a) Group 1 Modifications

In one aspect, the bacterial cell has a genetic modification which reduces expression of one or more endogenous genes selected from the group consisting of metJ, rzpD, yhjA, gtrS, ycdU, iscR, sspA and rph. For example, in one particular embodiment, the endogenous gene is metJ.

In another aspect, there is provided a bacterial cell which comprises genetic modifications reducing the expression of at least two endogenous genes.

In one embodiment, the genetic modifications reduce the expression of metJ and one or more other endogenous genes. In one particular embodiment, the other endogenous genes are relA and purT. In another particular embodiment, the other endogenous gene or genes is acre, acrA or both.

In another embodiment, the bacterial cell comprises genetic modifications which reduce expression of iscR and relA.

In another embodiment, the bacterial cell comprises genetic modifications which reduce expression of fabR and ygfF.

In another embodiment, the at least two endogenous genes bacterial cell comprises genetic modifications which reduce the expression of two or more of metJ, rzpD, yhjA, gtrS, ycdU, iscR, sspA and rph. In separate and specific embodiments, the bacterial cell comprises:
a first genetic modification which reduces the expression of metJ, and a second genetic modification which reduces the expression of a gene selected from rzpD, yhjA, gtrS, ycdU, iscR, sspA and rph;
a first genetic modification which reduces the expression of rzpD and a second genetic modification which reduces the expression of a gene selected from of metJ, yhjA, gtrS, ycdU, iscR, sspA and rph;
a first genetic modification which reduces the expression of yjhA and a second genetic modification which reduces the expression of a gene selected from of metJ, rzpD, gtrS, ycdU, iscR, sspA and rph;
a first genetic modification which reduces the expression of gtrS and a second genetic modification which reduces the expression of a gene selected from metJ, rzpD, yhjA, ycdU, iscR, sspA and rph;
a first genetic modification which reduces the expression of ycdU and a second genetic modification which reduces the expression of a gene selected from metJ, rzpD, yhjA, gtrS, iscR, sspA and rph;
a first genetic modification which reduces the expression of iscR and a second genetic modification which reduces the expression of a gene selected from metJ, rzpD, yhjA, gtrS, ycdU, sspA and rph, and, optionally, a third genetic modification which reduces the expression of relA;
a first genetic modification which reduces the expression of sspA and a second genetic modification which reduces the expression of a gene selected from metJ, rzpD, yhjA, gtrS, ycdU, iscR and rph; and
a first genetic modification which reduces the expression of rph and a second genetic modification which reduces the expression of a gene selected from metJ, rzpD, yhjA, gtrS, ycdU, iscR and sspA.

In one specific embodiment, either one or both of the first and second genetic modifications is a knock-out of the gene, optionally a deletion. In an alternative embodiment at least one of the first and second genetic modifications is a knock-down of the gene.

In one aspect, there is provided a bacterial cell according to any one of the preceding aspects and embodiments, wherein the genetic modification is a knock-down of the one or more endogenous genes, resulting in at least 25%, such as at least 50%, such as at least 75%, such as at least 90%, such as at least 95%, reduction in the level of mRNA encoded by the gene.

In one aspect, there is provided a bacterial cell according to any one of the preceding aspects and embodiments, wherein the genetic modification is a knock-down of the one or more endogenous genes, resulting in at least 25%, such as at least 50%, such as at least 75%, such as at least 90%, such as at least 95%, reduction in the level of protein encoded by the gene.

In one aspect, there is provided a bacterial cell according to any one of the preceding aspects and embodiments, wherein the genetic modification is a knock-out of the one or more endogenous genes.

Knock-down or knock-out of a gene can be accomplished by any method known in the art for bacterial cells, and include, e.g., lambda Red mediated recombination, P1 phage transduction, and single-stranded oligonucleotide recombineering/MAGE technologies (see, e.g., Datsenko and Wanner, 2000; Thomason et al., 2007; Wang et al., 2009). Typically, a knock-down of a gene can be accomplished by, for example, a mutation in the promoter region resulting in decreased transcription, a deletion or mutation in the coding region of the gene resulting in a reduced or fully or substantially eliminated activity of the protein, or by the presence of antisense sequences that interfere with transcription or translation of the gene, resulting in reduced expression of the protein. Preferably, the knocking-down of a gene results in at least 20% reduction in the expression level of the gene product in the bacterial cell, such as at least 30%, such as at least 40%, such as at least 50%, such as at least 60%, such as at least 70%, such as at least 80%, such as at least 90%, such as at least 95% or higher.

A knock-out of a gene includes elimination of a gene's expression, such as by introducing a mutation in the coding sequence and/or promoter so that at least a portion (up to and including all) of the coding sequence and/or promoter is disrupted, shifted or deleted, resulting in loss of expression of the protein, or expression only of a non-functional mutant or non-functional fragment of the endogenous protein. As used herein, the symbol “DELTA” denotes a deletion of an endogenous gene. Preferably, a knock-out of a gene results in 1% or less of the native gene product being detectable, such as no detectable gene product.

b) Group 2 Modifications

In certain embodiments, a mutant protein is expressed in the bacterial cell, e.g., from a mutated version of an endogenous gene, or from a transgene encoding the mutant protein. For example, the bacterial cell may comprise one or more mutations in at least one protein selected from NanK (SEQ ID NO:19), RpsA (SEQ ID NO:37), RpoA (SEQ ID NO:21); RpoB (SEQ ID NO:23), RpoC (SEQ ID NO:25), SpoT (SEQ ID NO:27), NusG (SEQ ID NO:29, Flu (SEQ ID NO:31), Lon (SEQ ID NO:33), and YgaH (SEQ ID NO:35), e.g., wherein the one or more mutations are selected from RpoC-L268K, RpoC-L268N, RpoC-L268Q, RpoC-L268R, RpoC-N309F, RpoC-N309S, RpoC-N309T, RpoC-N309W, RpoC-N309Y, RpoC-Y75A, RpoC-Y75C, RpoC-Y75S, RpoC-LTPVIE(822-827), RpoB-D549A, RpoB-D549G, RpoB-H447F, RpoB-H447S, RpoB-H447T, RpoB-H447W, RpoB-H447Y, RpoB-I1112S, RpoB-I1112T, RpoB-V931A, RpoB-V931I, RpoB-V931L, NanK-T128S, Flu-L642E, Flu-L642N, Flu-L642Q, Lon-1716S, Lon-1716T, YgaH-V39A, YgaH-V39I, YgaH-V39L, NusG-F144A, NusG-F144I, NusG-F144L, NusG-F144M, NusG-F144V, RpoA-D305A, RpoA-D305G, RpoA-G279A, RpoA-G279F, RpoA-G279I, RpoA-G279L, RpoA-G279M, RpoA-G279V, RpsA-D310A, RpsA-D310F, RpsA-D310I, RpsA-D310L, RpsA-D310M, RpsA-D310V, RpsA-G21A, RpsA-G21F, RpsA-G21I, RpsA-G21L, RpsA-G21M, RpsA-G21V, SpoT-I213A, SpoT-I213F, SpoT-I213L, SpoT-I213M, and SpoT-I213V. The bacterial cell may further comprise a Group 1 modification as set out herein.

In one embodiment, the bacterial cell comprises a Group 1 modification according to any aspect or embodiment herein as well as a mutation in one or more of NanK (e.g., NanK-T128S), RpsA (e.g., RpsA-G21V, RpsA-G21I, RpsA-G21L, RpsA-G21M, RpsA-G21F, RpsA-G21A), RpoB (e.g., RpoB-H447Y, RpoB-H447F, RpoB-H447W, RpoB-H447T, RpoB-H447S, RpoB-D549G, RpoB-D549A, RpoB-V931A, RpoB-V931L, RpoB-V931I, RpoB-I1112S, and/or RpoB-I1112T), RpoC (e.g., RpoC-L268R, RpoC-L268K, RpoC-L268Q, RpoC-L268N, RpoC-LTPVIE(822-827), RpoC-N309Y, RpoC-N309F, RpoC-N309W, RpoC-N309T, RpoC-N309S, RpoC-Y75C, RpoC-Y75S, and/or RpoC-Y75A), SpoT (e.g., SpoT-I213L, SpoT-I213V, SpoT-I213M, SpoT-I213A, or SpoT-I213F), NusG (e.g., NusG-F144V, NusG-F144I, NusG-F144L, NusG-F144M, or NusG-F144A), Flu (e.g., Flu-L642Q, Flu-L642N, or Flu-L642E), Lon (e.g., Lon-1716S or Lon-1716T), and YgaH (e.g., YgaH-V39A, YgaH-V39L, or YgaH-V39I) and/or a mutation in rph or the pyrE/rph intergenic region which increases the expression of PyrE, wherein the one or more mutations improve tolerance to an aliphatic polyol such as, e.g. 2,3-butanediol.

In one embodiment, the bacterial cell comprises a Group 1 modification according to any aspect or embodiment herein as well as a mutation in one or more of RpoA (e.g., RpoA-D305G, RpoA-D305A, RpoA-G279V, RpoA-G279I, RpoA-G279L, RpoA-G279M, RpoA-G279F, and/or RpoA-G279A) and RpsA (e.g., RpsA-D310V, RpsA-D310I, RpsA-D310L, RpsA-D310M, RpsA-D310F, or RpsA-D310A), and/or a mutation in rph or the pyrE/rph intergenic region which increases the expression of PyrE, wherein the one or more mutations improve tolerance to an aliphatic polyol such as, e.g., 1,2-propanediol.

In an alternative embodiment, the bacterial cell comprises a Group 1 modification according to any preceding aspect or embodiment as well as an upregulation of at least one of the endogenous genes NanK, RpsA, SpoT, NusG, PyrE, Flu, Lon, and YgaH, e.g., by transforming the bacterial cell with a transgene expressing the endogenous protein. To cause an up-regulation through increased expression of a protein, the copy number of a gene or genes encoding the protein may be increased. Alternatively, a strong and/or inducible promoter can be used to direct the expression of the gene, the gene being expressed either as a transient expression vehicle or homologously or heterologously incorporated into the bacterial genome. In another embodiment, the promoter, regulatory region and/or the ribosome binding site upstream of the gene can be altered to achieve the over-expression. The expression can also be enhanced by increasing the relative half-life of the messenger or other forms of RNA. Any one or a combination of these approaches can be used to effect upregulation of a desired target protein as needed.

In one embodiment, the bacterial cell comprises one or more mutations which increase(s) the expression level or activity of PyrE, optionally in combination with a Group 1 modification. E. coli K-12 MG1655 and W3110, plus their common ancestor strain W1485, are known to exhibit pyrimidine starvation in minimal media due to the presence a frameshift mutation occurring in rph relative to other E. coli strains (Jensen et al., 1993). This mutation disrupts the transcriptional/translational coupling required for efficient translation of pyrE, encoding orotate phosphoribosyltransferase in the pyrimidine biosynthesis pathway. Compensatory mutations that correct this deficiency are well-known in the art. One of these mutations is an 82 bp deletion near the 3′ terminus of rph, due to presence of two homologous GCAGAAGGC sequences flanking this 82 bp region (Conrad et al., 2009). In addition to the 82 bp deletion, a 1 bp deletion at coordinate 3815809 in the pyrE/rph intergenic region has previously been encountered in strains evolved for growth on a minimal glucose medium (LaCroix et al., 2015), and a wide array of other frameshift mutations, substitutions, and coding mutations near the 3′ terminus of rph were encountered in a short-term selection/evolution of combinatorial mutant libraries in minimal medium at an elevated temperature of 42° C. (Sandberg et al., 2014). Without being limited to theory, all of these mutations can serve the same function of increasing expression of PyrE, with the selective pressure for these mutations being even stronger in minimal media with particular imposed stresses (certain chemicals or heat) than in minimal media alone. In one embodiment, the bacterial cell comprises mutations in rph or the pyrE/rph intergenic region, such as, e.g., the 82 bp deletion near the 3′ terminus of rph, the 1 bp deletion in the intergenic region between pyrE and rph, or both.

In separate and specific embodiments, the bacterial cell comprises
a mutation which increases the expression of PyrE and a knock-out or knockdown of at least one of metJ, rzpD, yhjA, gtrS, ycdU, iscR, and sspA, such as metJ;
a mutation selected from NanK-T128S, RpsA-G21V, RpoB-H447Y, RpoC-L268R, RpoB-D549G, RpoB-V931A, RpoC-ΔTPVIE(822-827), RpoC-N309Y, SpoT-1213L, NusG-F144V, RpoC-Y75C, Flu-L642Q, RpoC-L268R, RpoB-11112S, Lon-1716S, and YgaH-V39A, or a conservative substitution of any thereof, and a knock-out or knockdown of at least one of metJ, rzpD, yhjA, gtrS, ycdU, iscR, sspA, such as metJ;
a mutation selected from RpoA-D305G, RpsA-D310V, and RpoA-G279V, or a conservative substitution of any thereof, and a knock-out or knockdown of at least one of metJ, rzpD, yhjA, gtrS, ycdU, iscR, sspA, such as metJ;
a mutation which increases the expression of PyrE and a knock-out or knockdown of metJ, relA, and purT in combination;
a mutation selected from NanK-T128S, RpsA-G21V, RpoB-H447Y, RpoC-L268R, RpoB-D549G, RpoB-V931A, RpoC-ΔTPVIE(822-827), RpoC-N309Y, SpoT-1213L, NusG-F144V, RpoC-Y75C, Flu-L642Q, RpoC-L268R, RpoB-11112S, Lon-1716S, and YgaH-V39A, or a conservative substitution of any thereof, and a knock-out or knockdown of metJ, relA, and purT in combination;
a mutation selected from RpoA-D305G, RpsA-D310V, and RpoA-G279V, or a conservative substitution of any thereof, and a knock-out or knockdown of metJ, relA, and purT in combination;
a mutation increasing the expression of PyrE and a knock-out or knockdown of a combination of metJ and acrB and/or acrA;
a mutation selected from NanK-T128S, RpsA-G21V, RpoB-H447Y, RpoC-L268R, RpoB-D549G, RpoB-V931A, RpoC-ΔTPVIE(822-827), RpoC-N309Y, SpoT-1213L, NusG-F144V, RpoC-Y75C, Flu-L642Q, RpoC-L268R, RpoB-11112S, Lon-1716S, and YgaH-V39A, or a conservative substitution of any thereof, and a knock-out or knockdown of a combination of metJ and acrB and/or acrA;
a mutation selected from RpoA-D305G, RpsA-D310V, and RpoA-G279V, or a conservative substitution of any thereof, and a knock-out or knockdown of a combination of metJ and acrB and/or acrA;
a mutation increasing the expression of PyrE and a knock-out or knockdown of iscR and relA in combination;
a mutation selected from NanK-T128S, RpsA-G21V, RpoB-H447Y, RpoC-L268R, RpoB-D549G, RpoB-V931A, RpoC-ΔTPVIE(822-827), RpoC-N309Y, SpoT-1213L, NusG-F144V, RpoC-Y75C, Flu-L642Q, RpoC-L268R, RpoB-11112S, Lon-1716S, and YgaH-V39A, or a conservative substitution of any thereof, and a knock-out or knockdown of iscR and relA in combination; or
a mutation selected from RpoA-D305G, RpsA-D310V, and RpoA-G279V, or a conservative substitution of any thereof, and a knock-out or knockdown of iscR and relA in combination.

In other separate and specific embodiments, the bacterial cell comprises
a mutant RpoC comprising a RpoC-L268R, RpoC-L268K, RpoC-L268Q or RpoC-L268N mutation and at least one genetic modification which reduces the expression of metJ, relA and purT;
a mutant RpoC comprising a RpoC-L268R, RpoC-L268K, RpoC-L268Q, or RpoC-L268N mutation and at least one genetic modification which reduces the expression of metJ and acrB, acrA or both;
a mutant RpoC comprising a RpoC-L268R, RpoC-L268K, RpoC-L268Q or RpoC-L268N mutation and at least one genetic modification which reduces the expression of metJ, relA, purT, and acrB, acrA or both;
a mutant RpoC comprising a RpoC-L268R, RpoC-L268K, RpoC-L268Q, or RpoC-L268N mutation and a mutant NanK comprising a NanK-T128S mutation, and at least one genetic modification which reduces the expression of metJ, relA, and purT, and acrB, acrA or both;
a mutant RpoC comprising a RpoC-L268R, RpoC-L268K, RpoC-L268Q, or RpoC-L268N mutation and a mutant NanK comprising a NanK-T128S mutation, and at least one genetic modification which reduces the expression of metJ and acrB, acrA or both;
a mutant RpoC comprising a RpoC-L268R, RpoC-L268K, RpoC-L268Q, or RpoC-L268N mutation and a mutant NanK comprising a NanK-T128S mutation, and at least one genetic modification which reduces the expression of metJ, relA, purT, and acrB, acrA or both;
a mutant RpoC comprising a RpoC-L268R, RpoC-L268K, RpoC-L268Q or RpoC-L268N mutation, a mutant NanK comprising a NanK-T128S mutation, and a mutant Flu comprising a Flu-L642Q, Flu-L642N, or Flu-L642E mutation, and at least one genetic modification which reduces the expression of metJ, relA, purT, elfD and acrB, acrA or both;
a mutant RpoB comprising a RpoB-11112S or RpoB-11112T mutation and at least one genetic modification which reduces the expression of iscR, relA, and acrB, acrA or both;
a mutant RpoB comprising a RpoB-11112S or RpoB-11112T mutation and at least one genetic modification which reduces the expression of iscR, relA, and acrB, acrA or both;
a mutant RpoB comprising a RpoB-11112S or RpoB-11112T mutation, and a mutant Lon comprising a Lon-1716S or Lon-1716T mutation, and at least one genetic modification which reduces the expression of iscR, relA, and acrB, acrA or both;
a mutant RpoB comprising a RpoB-11112S or RpoB-11112T mutation, a mutant Lon comprising a Lon-1716S or Lon-1716T mutation, and a mutant YgaH comprising a YgaH-V39A, YgaH-V39L, or YgaH-V39I mutation, and at least one genetic modification which reduces the expression of iscR, relA, and acrB, acrA or both; or
a mutant RpoB comprising a RpoB-11112S or RpoB-11112T mutation, a mutant Lon comprising a Lon-1716S or Lon-1716T mutation, a mutant YgaH comprising a YgaH-V39A, YgaH-V39L, or YgaH-V39I mutation, a genetic modification that increases the expression of PyrE, and at least one genetic modification which reduces the expression of iscR, relA, and acrB, acrA or both.

AcrB is part of a protein complex which includes AcrA (AcrAB-TolC), with TolC also serving as the outer membrane component of a number of other protein complexes. Accordingly, a knock-down or knock-out AcrA can result in the same phenotype as a knockdown or knock-out of AcrB. So, in any aspect or embodiment herein relating to a knock-down or knock-out of acrB, a knock-down or knock-out of acrA, or of acrA and acrB, can be used as an alternative.

In a specific embodiment, the bacterial cell comprises a knockdown or knockout of metJ, acrB, relA, and purT; and one or more Group 2 modifications selected from the group consisting of: a mutation in NanK such as NanK-T128S or a conservative substitution thereof; a mutation in RpoC such as RpoC-L268R or a conservative substitution thereof; and/or a mutation in Flu such as Flu-L642Q or a conservative substitution thereof. Optionally, the bacterial cell also comprises a knockdown or knockout of elfD.

In another specific embodiment, the bacterial cell comprises a knockdown or knockout of iscR and relA; and one or more Group 2 modifications selected from the group consisting of: a mutation in RpoB such as RpoB-I1112S or a conservative substitution thereof; a mutation in Lon such as 1716S or a conservative substitution thereof; a mutation in YgaH such as YgaH-V39A or a conservative substitution thereof; and/or a mutation increasing the expression of PyrE.

2) Production Pathways

Bacterial strains capable of producing diols and other polyols can be found, e.g., in the genera Enterobacter, Klebsiella, Serratia, Lactobacillus, Bacillus, Paenibacillus, Clostridia, Thermoanaerobacterium, Bacteroides, Pantoea, and Citrobacter sp. (Sabra et al., 2016; Jiang et al., 2014). For example, production of up to 150 g/L and 87.7 g/L 2,3-butanediol and 1,3-propanediol from glucose or glycerol, respectively, have been reported in Klebsiella pneumoniae and Clostridium IK124, respectively (Ma et al., 2009; Hirschmann et al., 2005).

In some aspects, however, the bacterial cell comprises a recombinant pathway for producing the diol or other polyol of interest. A recombinant pathway can, for example, be added to introduce the capability to produce the diol or other polyol in a bacterial cell which does not have a native pathway to do so, typically by transforming the cell with one or more heterologous enzymes catalyzing the desired reaction(s). Alternatively, in cases where the bacterial cell has native pathway for production of the diol or other polyol of interest, a recombinant pathway can nonetheless be introduced in order to increase the production yield, e.g., by overexpressing one or more native enzymes or transforming the cell with heterologous enzymes.

So, in one aspect, there is provided a bacterial cell with improved tolerance to at least one aliphatic polyol according to any aspect or embodiment described herein, wherein the bacterial cell further comprises a recombinant biosynthetic pathway for producing an aliphatic polyol of interest, such as, e.g., 2,3-butanediol, 1,2-propanediol, 1,4-butanediol, 1,3-propanediol, 1,2-butanediol, 1,5-pentanediol and/or 1,2-pentanediol. In a particular embodiment, the bacterial cell further comprises a recombinant biosynthetic pathway for producing 2,3-butanediol. In another particular embodiment, the bacterial cell further comprises a recombinant biosynthetic pathway for producing 1,2-propanediol. In another particular embodiment, the bacterial cell further comprises a recombinant biosynthetic pathway for producing 1,4-butanediol. In another particular embodiment, the bacterial cell further comprises a recombinant biosynthetic pathway for producing 1,3-propanediol. In another particular embodiment, the bacterial cell further comprises a recombinant biosynthetic pathway for producing 1,2-butanediol. In another particular embodiment, the bacterial cell further comprises a recombinant biosynthetic pathway for producing 1,5-pentanediol. In another particular embodiment, the bacterial cell further comprises a recombinant biosynthetic pathway for producing 1,2-pentanediol.

In principle, any such recombinant biosynthetic pathway which is known in the art can be introduced into the cell by standard recombinant technologies. Biosynthetic pathways suitable for production of diols in bacteria are well-known in the art and have been described by, e.g., Xu et al. (2014), Jiang et al. (2014), Sabra et al. (2016), Saxena et al. (2010), Altaras and Cameron (2000), Clomburg and Gonzalez (2011), Zhu et al. (2016), Jain et al. (2015), Yim et al. (2011), Nakamura and Whited (2003), and Kataoka et al. (2013). Some specific, preferred pathways are, however, exemplified below and in Example 1, the section entitled “Biological production of 1,2-propanediol and 2,3-butanediol”. It is to be understood that, when a specific enzyme of these biosynthetic pathways is mentioned by name such as, e.g., “acetolate synthase”, the enzyme may be any characterized and sequenced enzyme, from any species, that have been reported in the literature so long as it provides the desired activity. In some embodiments, the enzyme is an overexpressed gene which is native to the host cell used. In some embodiments, the enzyme is a functionally active fragment or variant of an enzyme which is heterologous or native to the host cell. Also, in some embodiments, the recombinant biosynthetic pathway comprises a knock-down or a knock-out of one or more genes, typically for the purpose of avoiding competing reactions reducing the yield of the desired aliphatic polyol.

So, in one embodiment, the biosynthetic pathway is for producing 2,3-butanediol from the cellular glycolytic intermediate pyruvate, and comprises genes, optionally overexpressed and/or heterologous, encoding:
an acetolactate synthase, e.g., BudB from Enterobacter cloacae, catalyzing the conversion of two pyruvate molecules to acetolactate;
an acetolactate decarboxylase, e.g., BudA from Enterobacter cloacae, catalyzing the conversion of acetolactate to acetoin; and
a 2,3-butanediol dehydrogenase (or acetoin reductase), e.g., BudC from Enterobacter cloacae, catalyzing the conversion of acetoin to 2,3-butanediol

Typically, the native genes adhE, gloA, IdhA, tpiA, and/or zwf are knocked-down or -out to reduce lactate production, ethanol production, and carbon flux into the pentose phosphate pathway.

In another embodiment, the biosynthetic pathway is for producing 2,3-butanediol from the cellular glycolytic intermediate pyruvate, and comprises genes, optionally overexpressed and/or heterologous, encoding:
an acetolactate synthase, e.g., BudB from Enterobacter cloacae, catalyzing the conversion of two pyruvate molecules to acetolactate, which spontaneously decarboxylates to diacetyl;
a diacetyl reductase, e.g., BudC from Klebsiella pneumoniae, catalyzing the conversion of diacetyl to acetoin; and
a 2,3-butanediol dehydrogenase (or acetoin reductase), e.g., BudC from Enterobacter cloacae, catalyzing the conversion of acetoin to 2,3-butanediol.

In another embodiment, the biosynthetic pathway is for producing 2,3-butanediol from the cellular glycolytic intermediate pyruvate, and comprises genes, optionally overexpressed and/or heterologous, encoding:
an acetolactate synthase, e.g., BudB from Enterobacter cloacae, catalyzing the conversion of two pyruvate molecules to acetolactate;
an acetolactate decarboxylase, e.g., BudA from Enterobacter cloacae, catalyzing the conversion of acetolactate to acetoin;
an acetoin dehydrogenase, e.g., BudC from Klebiella pneumoniae, catalyzing the conversion of acetoin to diacetyl;
an acetylacetoin synthase, e.g., from Bacillus licheniformis, catalyzing the conversion of two diacetyl molecules to acetylacetoin;
an acetylacetoin reductase, e.g., from Bacillus licheniformis, catalyzing the conversion of acetylacetoin to acetylbutanediol; and
an acetylbutanediol reductase, e.g., from Bacillus licheniformis, catalyzing the conversion of acetylbutanediol to 2,3-butanediol.

Optionally, the biosynthetic pathway does not constitute an acetolactate decarboxylase nor an acetoin dehydrogenase, and acetolactate is instead spontaneously converted to acetoin.

In one embodiment, the biosynthetic pathway is for producing 1,2-propanediol from the cellular glycolytic intermediate dihydroxyacetone phosphate, and comprises genes, optionally overexpressed and/or heterologous, encoding:
a methylglyoxal synthase, e.g., MgsA from E. coli, catalyzing the conversion of dihydroxyacetone phosphate to methylglyoxal;
a methylglyoxal reductase or glycerol dehydrogenase, e.g., GlyD and GlyH from E. coli, catalyzing the conversion of methylglyoxal to lactaldehyde; and
a lactaldehyde reductase or 1,2-propanediol reductase, e.g., FucO from E. coli, catalyzing the conversion of lactaldehyde to 1,2-propanediol.

Optionally, native lactate dehydrogenases which convert pyruvate to lactate, such as (in E. coli), LdhA, can be deleted (Altaras and Cameron, 2000).

In another embodiment, the biosynthetic pathway is for producing 1,2-propanediol from the cellular glycolytic intermediate dihydroxyacetone phosphate, and comprises genes, optionally overexpressed and/or heterologous, encoding:
a methylglyoxal synthase, e.g., MgsA from E. coli, catalyzing the conversion of dihydroxyacetone phosphate to methylglyoxal;
an aldehyde oxidoreductase, e.g. YqhD from E. coli, catalyzing the conversion of methylglyoxal to acetol; and
a glycerol reductase, e.g., GlyD and GlyH from E. coli, catalyzing the conversion of acetol to 1,2-propanediol.

Optionally, native lactate dehydrogenases which convert pyruvate to lactate, such as (in E. coli), LdhA, can be deleted (Altaras and Cameron, 2000).

In another embodiment, the biosynthetic pathway is for producing 1,2-propanediol from the cellular glycolytic intermediate pyruvate, and comprises genes, optionally overexpressed and/or heterologous, encoding:
a lactate dehydrogenase, e.g., LdhA from E. coli, catalyzing the conversion of pyruvate to lactate;
a lactaldehyde dehydrogenase, e.g. AldA from E. coli, catalyzing the conversion of lactate to lactaldehyde; and
a lactaldehyde reductase or 1,2-propanediol reductase, e.g., FucO from E. coli, catalyzing the conversion of lactaldehyde to 1,2-propanediol.

In another embodiment, the biosynthetic pathway is for producing 1,2-propanediol from the cellular glycolytic intermediate pyruvate, and comprises genes, optionally overexpressed and/or heterologous, encoding:
a methylglyoxal synthase, e.g., MgsA from E. coli, catalyzing the conversion of dihydroxyacetone phosphate to methylglyoxal;
a Type I glyoxylase, e.g. GloA from E. coli, catalyzing the conversion of methylglyoxal and glutathione to (S)-lactoylglutathione;
a Type II glyoxylase, e.g. GloB from E. coli, catalyzing the conversion of (S)-lactoylglutathione to lactate and glutathione;
a lactaldehyde dehydrogenase, e.g. AldA from E. coli, catalyzing the conversion of lactate to lactaldehyde; and
a lactaldehyde reductase or 1,2-propanediol reductase, e.g., FucO from E. coli, catalyzing the conversion of lactaldehyde to 1,2-propanediol.

Optionally, native lactate dehydrogenases which convert pyruvate to lactate, such as (in E. coli), LdhA, can be deleted (Altaras and Cameron, 2000).

In another embodiment, the biosynthetic pathway is for producing 1,2-propanediol from the cellular glycolytic intermediate pyruvate, and comprises genes, optionally overexpressed and/or heterologous, encoding:
a lactate dehydrogenase, e.g., LdhA from E. coli, catalyzing the conversion of pyruvate to lactate;
a CoA transferase, e.g., Pct from Clostridium propionicum DSM 1682, catalyzing the conversion of lactate and CoA to lactoyl-CoA;
an aldehyde dehydrogenase, e.g., a CoA-dependent succinate semialdehyde dehydrogenase (PdcD) from Yersinia enterocolitica subsp. enterocolitica 8081, catalyzing the conversion of lactoyl-CoA to lactaldehyde; and
an alcohol dehydrogenase, e.g. a 3-hydroxypropionate dehydrogenase (MmsB) from Bacillus cereus ATCC 14579, catalyzing the conversion of lactaldehyde to 1,2-propanediol.

In one embodiment, the biosynthetic pathway is for producing 1,4-butanediol from the cellular tricarboxylic acid intermediate succinate, and comprises genes, optionally overexpressed and/or heterologous, encoding:
a succinyl-CoA synthetase, e.g., SucCD from E. coli, catalyzing the conversion of succinate to succinyl-CoA;
a CoA-dependent succinate semialdehyde dehydrogenase, e.g., SucD from E. coli, catalyzing the conversion of succinyl-CoA to succinyl semialdehyde;
a 4-hydroxybutyrate dehydrogenase, e.g., 4HBd from Porphyromonas gingivalis, catalyzing the conversion of succinyl semialdehyde to 4-hydroxybutryrate;
a 4-hydroxybutyryl-CoA transferase, e.g., Cat2 from Porphyromonas gingivalis, catalyzing the conversion of 4-hydroxybutyrate to 4-hydroxybutyryl-CoA;
a 4-hydroxybutyryl-CoA reductase, e.g., the bifunctional enzyme 0256 from Clostridium beijerinckii, catalyzing the conversion of 4-hydroxybutyryl-CoA to 4-hydroxybutyrylaldehyde; and
an alcohol dehydrogenase, e.g., the bifunctional enzyme 0256 from Clostridium beijerinckii, catalyzing the conversion of 4-hydroxybutryrylaldehyde to 1,4-butanediol.

Optionally, native malate dehydrogenase, such as (in E. coli), Mdh, can be deleted. Optionally, one or more subunits of a global regulator of gene expression under microaerobic and/or aerobic conditions, such as (in E. coli), ArcAB, can be deleted. Optionally, native lactate and/or alcohol dehydrogenases, and/or pyruvate formate lyase, such as (in E. coli) LdhA, AdhE, and PfIB, can be deleted. Optionally, pyruvate dehydrogenase can be modified by deleting the native lipoamide dehydrogenase (e.g., LpdA in E. coli) and heterologously expressing an anaerobically functional LpdA such as from Klebsiella pneumoniae. The heterologously expressed LpdA can optionally harbor a mutation reducing NADH sensitivity, such as D354K. Optionally, tricarboxylic acid cycle flux can increased by introducing a mutation to reduce NADH inhibition of citrate synthase, e.g., by introducing a GltA-R163L mutation to E. coli GltA. Optionally, an α-ketoglutarate decarboxylase can be overexpressed, e.g., SucA from E. coli, to additionally convert the tricarboxylic acid cycle intermediate α-ketoglutarate to succinyl semialdehyde (Yim et al., 2011).

In one embodiment, the biosynthetic pathway is for producing 1,3-propanediol from the cellular glycolytic intermediate dihydroxyacetone phosphate, and comprises genes, optionally overexpressed and/or heterologous, encoding:
a glycerol-3-phosphate dehydrogenase and glycerol-3-phosphate phosphatase, e.g., DAR1 and GPP2 from Saccharomyces cerevisiae, catalyzing the conversion of dihydroxyacetone phosphate to glycerol;
a glycerol dehydratase, e.g., DhaB1, DhaB2, and DhaB3 from Klebsiella pneumoniae, catalyzing the conversion of glycerol to 3-hydroxypropionaldehyde; and
an aldehyde oxidoreductase, e.g., YqhD from E. coli, catalyzing the conversion of 3-hydroxypropionaldehyde to 1,3-propanediol.

Optionally, PEP-dependent glucose transport is eliminated via deletion of one or more genes in glucose-specific PTS enzyme II, e.g., PtsG in E. coli, and an ATP-dependent glucose transport system composed of galactose permease (e.g., GaIP in E. coli) and glucokinase (e.g., Glk in E. coli) are overexpressed or heterologously expressed. Optionally, glyceraldehyde-3-phosphate dehydrogenase (e.g., Gap in E. coli), is downregulated (Nakamura and Whited, 2003).

In one embodiment, the biosynthetic pathway is for producing 1,3-butanediol from the cellular intermediate acetyl-CoA, and comprises genes, optionally overexpressed and/or heterologous, encoding:
a 3-ketothiolase, e.g., PhaA from Ralstonia eutropha NBRC 102504, catalyzing the conversion of acetyl-CoA to acetoacetyl-CoA;
an acetoacetyl-CoA reductase, e.g., PhaB from Ralstonia eutropha NBRC 102504, catalyzing the conversion of acetoacetyl-CoA to 3-hydroxybutyryl-CoA;
a butyraldehyde dehydrogenase, e.g., Bld from Clostridium saccharoperbutylacetonicum ATCC 27012, catalyzing the conversion of 3-hydroxybutyryrl-CoA to 3-hydroxybutyraldehyde; and
an aldehyde-alcohol dehydrogenase, e.g., AdhE from E. coli, catalyzing the conversion of 3-hydroxybutyraldehyde to 1,3-butanediol.

In one embodiment, 1,5-pentanediol is produced from glutaric acid, optionally via glutaryl-CoA, via reduction of the 1- and 5-carboxylic acids to alcohols. Pathways describing the production of glutaric acid from the intracellular amino acid L-lysine have been described (Adkins et al., 2013; Park et al., 2013). Biosynthesis of the glutaryl-CoA intermediate has been described by Cheong et al., 2016.

3) Processes

In one aspect, there is provided a process for preparing a recombinant bacterial cell, e.g., an E. coli cell. Also provided is a process for improving the tolerance of a bacterial cell, e.g., an E. coli cell, to a diol or other polyol. Also provided is a method of identifying a bacterial cell which is tolerant to at least one diol or other polyol. Also provided is a process for preparing a recombinant bacterial cell, e.g., an E. coli cell, for producing a diol or other polyol.

These processes may comprise one or more steps of genetically modifying a bacterial cell to knock-down or knock-out one or more endogenous genes of any aspect or embodiment of the Group 1 modifications and/or introducing one or more mutations in the endogenous protein(s) or gene(s) of any Group 2 aspect or embodiment. This can be achieved by, e.g., transforming the bacterial cell with genetic constructs, e.g., vectors, antisense nucleic acids or siRNA, which result, e.g., in the knock-out or knock-down of a gene, introduce a mutation into an endogenous gene, or which encode the mutated protein from a transgene.

The genetic constructs, particularly vectors, can also comprise suitable regulatory sequences, typically nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters (e.g., constitutive promoters or inducible promoters), translation leader sequences, introns, polyadenylation recognition sequences, RNA processing sites, effector binding sites and stem-loop structures.

Alternatively, bacterial cells can be exposed to selection pressure (as described in the Examples) or to conditions which introduce random mutations in endogenous genes, and bacterial cells which comprise one or more Group 1 and/or Group 2 modifications according to any preceding aspects and embodiments can then be identified. Typically, this involves preparing a population of the genetically modified bacterial cell, having different Group 1 and/or Group 2 modifications, and then selecting from this population any bacterial cell which has an improved tolerance to the diol or other polyol, e.g., an aliphatic diol or other polyol.

In one specific embodiment, the Group 1 modification is a knock-down or knock-out of one or more endogenous genes selected from metJ, rzpD, yhjA, gtrS, ycdU, iscR, sspA and rph or, e.g., a knock-down or knock-out of metJ in combination with relA and purT or with acre and/or acrA, and/or a knock-down or knock-out of iscR and relA. In one specific embodiment, the Group 2 modification is a mutation in at least one endogenous protein or gene selected from NanK, RpsA, RpoB, RpoC, SpoT, NusG, Flu, Lon, or YgaH, such as e.g., NanK-T128S, RpoA-D305G, RpoA-D305A, RpsA-D310V, RpsA-D310I, RpsA-D310L, RpsA-D310M, RpsA-D310F, RpsA-D310A, RpoA-G279V, RpoA-G279I, RpoA-G279L, RpoA-G279M, RpoA-G279F, RpoA-G279A, RpsA-G21V, RpsA-G21I, RpsA-G21L, RpsA-G21M, RpsA-G21F, RpsA-G21A, RpoB-H447Y, RpoB-H447F, RpoB-H447W, RpoB-H447T, RpoB-H447S, RpoC-L268R, RpoC-L268K, RpoC-L268Q, RpoC-L268N, RpoB-D549G, RpoB-D549A, RpoB-V931A, RpoB-V931L, RpoB-V931I, RpoC-LTPVIE(822-827), RpoC-N309Y, RpoC-N309F, RpoC-N309W, RpoC-N309T, RpoC-N309S, SpoT-I213L, SpoT-I213V, SpoT-I213M, SpoT-I213A, SpoT-I213F, NusG-F144V, NusG-F144I, NusG-F144L, NusG-F144M, NusG-F144A, RpoC-Y75C, RpoC-Y75S, RpoC-Y75A, Flu-L642Q, Flu-L642N, Flu-L642E, RpoB-I1112S, RpoB-I1112T, Lon-1716S, Lon-1716T, YgaH-V39A, YgaH-V39L, or a YgaH-V39I mutation and/or a mutation which increases the expression of PyrE, such as, e.g. a mutation in rph or the pyrE/rph intergenic region.

In one embodiment, the process comprises genetically modifying the bacterial cells, e.g., the E. coli cells, to express a mutant NanK, RpoC, Flu, RpoB, Lon, YgaH, such as, e.g., NanK-T128S, RpoC-L268R, Flu-L642Q, RpoB-I1112S, Lon-1716S, and/or YgaH-V39A mutation, or a conservative substitution of any thereof, and/or a mutation which increases the expression of PyrE.

The processes may further comprise
a step of selecting any bacterial cell which has an improved tolerance to a diol or other polyol at a predetermined concentration, such as at least 1% v/v or higher, such as at least 1.5% v/v or higher, such as at least 3% v/v or higher, such as at least 5% v/v or higher, such as at least 6% v/v or higher, such as at least 7% v/v or higher, such as at least 8% v/v or higher, such as at least 10% v/v or higher;
an optional step of introducing a recombinant biosynthetic pathway for producing the diol or other polyol; or
both of the above steps, in any order.

In one embodiment, the diol is 2,3-butanediol, and the predetermined concentration is at least 1% v/v or higher, such as at least 1.5% v/v or higher, such as at least 3% v/v or higher, such as at least 5% v/v or higher, such as at least 6% v/v or higher, such as at least 7% v/v or higher, such as at least 10% v/v or higher. In one embodiment, the diol is 1,2-propanediol, and the predetermined concentration is at least 1% v/v or higher, such as at least 1.5% v/v or higher, such as at least 3% v/v or higher, such as at least 5% v/v or higher, such as at least 6% v/v or higher, such as at least 7% v/v or higher, such as at least 7.5% v/v or higher, such as at least 8% v/v or higher, such as at least 10% v/v or higher. In one embodiment, the diol is 1,5-pentanediol, and the predetermined concentration is at least 0.5% v/v or higher, such as at least 1% v/v or higher, such as at least 2% v/v or higher, such as at least 3% v/v or higher, such as at least 5% v/v or higher, such as at least 6% v/v or higher, such as at least 7% v/v or higher, such as at least 7.5% v/v or higher, such as at least 8% v/v or higher, such as at least 10% v/v or higher. In one embodiment, the diol is 1,2-pentanediol, and the predetermined concentration is at least 0.5% v/v or higher, such as at least 1% v/v or higher, such as at least 2% v/v or higher, such as at least 3% v/v or higher, such as at least 5% v/v or higher, such as at least 6% v/v or higher, such as at least 7% v/v or higher, such as at least 7.5% v/v or higher, such as at least 8% v/v or higher, such as at least 10% v/v or higher.

In a particular embodiment, the predetermined concentration is at most 7%, such as at most 8%, such as at most 9%, such as at most 10%, such as at most 15%, such as at most 20%.

Assays for assessing the tolerance of a modified bacterial cell to the diol or other polyol typically evaluate the growth rate, lag time, or both, of the bacterial cell at predetermined concentrations for the diol or other polyol in question, typically as compared to a control. Preferably, the control is the native or unmodified parent cell or strain, and an improved tolerance is identified as an improved growth rate, a reduced lag-time or both. For example, an improved growth rate can be at least 5%, such as at least 10%, such as at least 20%, such as at least 50%, such as at least 75% higher than that of the control, while a reduced lag time can be at least 10%, such as at least 20%, such as at least 50%, such as at least 75%, such as at least 90% shorter than that of the control. Specific assays are described, in detail, in the Examples.

Also provided is a method of producing a diol or other polyol, comprising culturing the bacterial cell obtained by any one of these methods, or the bacterial cell of any preceding aspect or embodiment, under conditions where the diol or other polyol is produced. Typically, these conditions include the presence of a suitable carbon source or mixes of different suitable carbon sources. Non-limiting examples of suitable carbon sources include, e.g., sucrose, D-glucose, D-xylose, L-arabinose, glycerol; raw carbon feedstocks such as crude glycerol and cane syrup; as well as hydrolysates produced from cellulosic or lignocellulosic materials. For further details see, e.g., Sabra et al., 2016; Clomburg et al., 2011; Jain et al., 2015; Li et al., 2015; Jiang et al., 2014; and Xu et al., 2014.

The inventors have further discovered that methionine supplementation can improve endogenous production of diols in diol-overproducing strains during fermentation. In particular, robust growth of K-12 MG1655 in 6% 2,3-butanediol or 8% 1,2-propanediol was significantly restored by the addition of methionine, with a growth rate approaching that of evolved strains in such media, whereas evolved strains did not have a significantly enhanced growth rate with the addition of methionine.

Accordingly, in one embodiment, there is provided a method for producing an aliphatic diol, comprising culturing a bacterial cell capable of producing the aliphatic diol in the presence of a carbon source and adding methionine to the cultivation medium, wherein the concentration of the added methionine is at least about 0.004 g L⁻¹ gDCW⁻¹ (gDCW=grams dry cell weight), such as at least about 0.007 g L⁻¹ gDCW⁻¹, such as at least 0.015 g L⁻¹ gDCW⁻¹, such as at least about 0.03 g L⁻¹ gDCW⁻¹, such as at least about 0.07 g L⁻¹ gDCW⁻¹, such as at least about 0.2 g L⁻¹ gDCW⁻¹. In a particular embodiment, the added methionine concentration is at most 0.03 g L⁻¹ gDCW⁻¹, such as at most 0.07 g L⁻¹ gDCW⁻¹, such as at most 0.2 g L⁻¹ gDCW⁻¹. In some embodiments, the added methionine concentration is in the range from about 0.0004 to about 0.2, such as from about 0.007 to about 0.2, such as from about 0.015 to about 0.2, such as from about 0.03 to about 0.2, such as from about 0.07 to about 0.2 g L⁻¹ gDCW⁻¹. Optionally, the bacterial cell may comprise one or more genetic modifications according to any aspect or embodiment described herein. In one embodiment, the medium comprises no more than 10, such as no more than 8, such as no more than 6, such as no more than 5, such as no more than 4 other natural amino acids, e.g., selected from alanine, arginine, asparagine, aspartic acid, cysteine, glutamine, glutamic acid, glycine, histidine, isoleucine, leucine, lysine, phenylalanine, proline, serine, threonine, tryptophan, tyrosine and valine at a biologically relevant level, e.g., at a concentration of at least 0.0002 g L⁻¹ gDCW⁻¹. Optionally, the method further comprises isolating the aliphatic diol.

4) Compositions

A bacterial cell which has an increased tolerance to a diol or other polyol can be useful for preparing producer cells for the production of the diol or other polyol. Bacterial cells according to the invention may have an increased growth rate, an decreased lag time, or both. For example, the bacterial cell may have Group 1 and/or Group 2 modifications providing for an increased growth rate, a reduced lag time, or both, of the cell in at least one of a propanediol, butanediol, pentanediol or a hexanediol, e.g., 2,3-butanediol, 1,2-propanediol; 1,4-butanediol, 1,3-propanediol, 1,2-butanediol, 1,5 pentanediol and/or 1,2-pentanediol, such as in 2,3-butanediol, 1,2-propanediol, or both.

In one aspect, there is provided a composition of a plurality of bacterial cells according to any aspect or embodiment described herein, e.g., an in vitro culture of such bacterial cells, optionally in a suitable culture medium and/or a chemically-defined medium comprising a carbon source. In one embodiment, the composition is substantially homogenous with respect to the bacterial cells.

In one aspect, there is provided a composition comprising a plurality of bacterial cells according to any preceding aspect or embodiment and a diol or other polyol. In one embodiment, the diol or other polyol is present at a concentration at which the genetic modification(s) and/or mutant(s) comprised in the bacterial cells results in an improved tolerance as compared to the parent bacterial cells, e.g., wild-type or native bacterial cells.

The concentrations at which bacterial cells according to the invention have improved tolerance are shown in Example 1, e.g., in “Cross-compound tolerance testing”. Typically, the concentration of the a diol or other polyol is at least 1% v/v or higher, such as at least 1.5% v/v or higher, such as at least 3% v/v or higher, such as at least 5% v/v or higher, such as at least 6% v/v or higher, such as at least 7.5% v/v or higher, such as at least 10% v/v or higher, such as at least 20% v/v or higher; such as at least 30% v/v or higher; such as in the range of 1% to 30% v/v, such as in the range of 2% to 20% v/v; such as in the range of 5% to 15% v/v or 5 to 10% v/v.

In one embodiment, the composition comprises 2,3-butanediol. In one embodiment, the composition comprises 1,2-propanediol. In one embodiment, the composition comprises 1,5-pentanediol. In one embodiment, the composition comprises 1,2-pentanediol. In one embodiment, the composition comprises 1,4-butanediol. In one embodiment, the composition comprises 1,3-propanediol. In one embodiment, the composition comprises 1,2-butanediol.

As described in Example 1; “Cross-compound tolerance testing”, some of the genetic modifications according to the invention also confer tolerance to other chemicals, such as to other polyols or diols, to hexanoate and/or to p-coumarate. Accordingly, in one embodiment, there is provided a composition comprising
hexanoate at a concentration of at least 0.1 g/L, such as at least 1 g/L, such as at least 5 g/L, such as at least 10 g/L, such as at least 20 g/L, or p-coumarate at a concentration of at least 1 g/L, such as at least 2.5 g/L, such as at least 5 g/L, such as at least 7.5 g/L, such as at least 15 g/L; and
a plurality of bacterial cells according to any preceding aspect or embodiment.

Preferably, the bacterial cells are of the Escherichia, Lactobaccillus, Lactococcus, Corynebacterium, Bacillus, Ralstonia, or Pseudomonas genera, such as, e.g., E. coli cells, and comprise
a) at least one genetic modification which reduces expression of an endogenous gene selected from the group consisting of metJ, rzpD, yhjA, gtrS, ycdU, iscR, sspA and rph, or a combination of any thereof;
b) genetic modifications which reduce expression of metJ, relA and purT,
c) genetic modifications which reduce expression of metJ and acrB and/or acrA, or
d) genetic modifications which reduce expression of iscR and relA.

Such bacterial cells may further comprise one or more Group 2 modifications as described in any aspect or embodiment herein.

Assays for assessing the tolerance of a modified bacterial cell to a diol or other polyol typically evaluate the growth rate, lag time, or both, of the bacterial cell at one or more predetermined concentrations of the compound, typically as compared to a control (e.g., no compound). The predetermined concentrations(s) could be, for example, 6% v/v, 7% v/v or 8% v/v. Preferably, the control is the native or unmodified parent cell or strain, and an improved tolerance is identified as an improved growth rate, a reduced lag-time or both. For example, an improved growth rate can be at least 5%, such as at least 10%, such as at least 20%, such as at least 50%, such as at least 75% higher than that of the control, while a reduced lag time can be at least 10%, such as at least 20%, such as at least 50%, such as at least 75%, such as at least 90% shorter than that of the control. Specific assays are described, in detail, in the Examples.

5) Bacterial Cells

Also provided are strains, clones and other progeny of the bacterial cells of these and other aspects and embodiments, as well as cell cultures of such bacterial cells or strains. Typically, as used herein, a “strain” typically refers to a group of cells which are descendants of a initial single colony of parent cells whereas a “clone” is a group of cells which are the descendants of an initial genetically modified single parent cell.

Non-limiting examples of bacterial cells suitable for modification according to any one of the aspects and embodiments described herein include bacteria of the Escherichia, Enterobacter, Klebsiella, Lactobaccillus, Lactococcus, Corynebacterium, Bacillus, Ralstonia, Paenibacillus, Clostridia, Citrobacter sp. or Pseudomonas genera, such as from the Escherichia, Lactobacillus, Lactococcus, Corynebacterium, Bacillus, Ralstonia, or Pseudomonas genera. In one embodiment, the bacterial cell is an E. coli cell, such as a cell of the commercially available and/or fully characterized strains K-12 MG1655, BW25113, BL21, BL21(DE3), K-12 W3110, W, JM109, or Crooks (ATCC 8739). In a specific embodiment, the bacterial cell is derived from an E. coli K12 strain. In another embodiment, the bacterial cell is a Lactobacillus cell, such as a cell of the commercially available and/or fully characterized strains Lactobacillus plantarum 3DM1, Lactobacillus plantarum WCFS1, and Lactobacillus plantarum NCIMB 8826. In another embodiment, the bacterial cell is a Lactococcus cell, such as a cell of the commercially available and/or fully characterized strains Lactococcus lactis lactis CV56, Lactococcus lactis lactis NIZO B40, and Lactococcus lactis cremoris NZ9000. In another embodiment, the bacterial cell is a Bacillus cell, such as a cell of the commercially available and/or fully characterized strains Bacillus subtilis 168 and Bacillus subtilis PY79. In one embodiment, the bacterial cell is a Pseudomonas cell, such as a cell of the commercially available and/or fully characterized strain Pseudomonas putida KT2440. In another embodiment, the bacterial cell is a Ralstonia cell, such as a cell of the commercially available and/or fully characterized strains Ralstonia eutropha H16 and Ralstonia eutropha JMP134. In another embodiment, the bacterial cell is a Corynebacterium cell, such as a cell of the commercially available and/or fully characterized strains 534 (ATCC 13032), K051, MB001, R, SCgG1, and SCgG2.

While aspect and embodiments relating to bacterial cells herein typically refer to genes or proteins according to their designation in E. coli, for bacterial cells of another family or species, it is within the level of skill in the art to identify the corresponding gene or protein, i.e., the ortholog and/or paralog, in the other family or species, typically by identifying sequences having moderate or high homology to the E. coli sequence, optionally taking the function of the protein expressed by the gene and/or the locus of the gene in the genome into account. Table 2A below sets out the function of the protein encoded by each specific gene, the corresponding E.C. number (if applicable), its locus in the E. coli K-12 MG1655 genome and the SEQ ID number of the coding or non-coding sequence and, where applicable, the encoded amino acid sequence.

Table 2B below sets out some examples of homologs or orthologs in selected organisms, identified in a preliminary and non-limiting analysis. Indeed, homologs or orthologs of these proteins exist also in other bacteria, and other homologs or orthologs not identified in this preliminary search can exist in the species listed in Table 2B. The skilled person is well-familiar with different searching and/or screening methods for identifying homologs or orthologs across different species. To briefly summarize some of the preliminary findings in Table 2B:
RelA, PurT, YfgF, and PyrE are widely conserved and were identified in all organisms.
AcrB homologs or orthologs were identified in the Gram-negative species and Bacillus subtilis.
Rph was found to be conserved in all organisms with the exception of Lactococcus lactis.
FabR, RzpD, and YhjA had the longest alignments with Pseudomonas putida proteins, with other more partial alignments to FabR annotated as transcriptional regulators in all other organisms.
IscR was found in Gram-negative organisms and Bacillus subtilis, with other more partial alignments annotated as transcriptional regulators in other organisms.
SspA was found to be conserved in Gram-negative organisms.
GtrS, YcdU, Meti, Flu (with the exception of partial conservation in Pseudomonas putida), and YgaH were not widely conserved.
RpoA, RpoB, RpoC, SpoT, RpsA and NusG were found to be widely conserved in all organisms.
Lon was found to be conserved in Gram-negative organisms.

TABLE 2
Protein function and Locus IDs

| E. coli gene | | E.C. | | |
|---|---|---|---|---|
| designation | Protein function | number | Locus ID | SEQ ID NO: |
| metJ | MetJ transcriptional repressor | N/A | b3938 | 1 |
| rzpD | DLP12 prophage; predicted | N/A | b0556 | 2 |
| | murein endopeptidase | | | |
| yhjA | predicted cytochrome C | N/A | b3518 | 3 |
| | peroxidase | | | |
| gtrS | CPS-53 (KpLE1) prophage; | N/A | b2352 | 4 |
| | predicted inner membrane | | | |
| | protein | | | |
| ycdU | predicted inner membrane | N/A | b1029 | 5 |
| | protein | | | |
| iscR | IscR DNA-binding transcriptional | N/A | b2531 | 6 |
| | dual regulator | | | |
| sspA | stringent starvation protein A | N/A | b3229 | 7 |
| Rph | ribonuclease PH | 2.7.7.56 | b3643 | 8 |
| relA | GDP pyrophosphokinase/GTP | 2.7.6.5 | b2784 | 9 |
| | pyrophosphokinase | | | |
| purT | phosphoribosylglycinamide | 2.1.2.—; | b1849 | 10 |
| | formyltransferase 2 | 2.7.2.1 | | |
| acrB | AcrAB-ToIC multidrug efflux | N/A | b0462 | 11 |
| | system - permease subunit | | | |
| acrA | AcrAB-ToIC multidrug efflux | N/A | b0463 | 12 |
| | system - membrane fusion | | | |
| | protein | | | |
| fabR | FabR DNA-binding | N/A | b3963 | 13 |
| | transcriptional repressor | | | |
| ygfF | cyclic di-GMP phosphodiesterase | 3.1.4.52 | b2503 | 14 |
| pyrE | Orotate | 2.4.2.10 | b3642 | 15 (DNA) |
| | phosphoribosyltransferase | | | 16 (protein) |
| pyrE/rph | — | — | — | 17 |
| intergenic | | | | |
| region | | | | |
| nanK | N-acetylmannosamine kinase | 2.7.1.60 | b3222 | 18 (DNA) |
| | | | | 19 (protein) |
| rpoA | RNA polymerase subunit α | 2.7.7.6 | b3295 | 20 (DNA) |
| | | | | 21 (protein) |
| rpoB | RNA polymerase subunit β | 2.7.7.6 | b3987 | 22 (DNA) |
| | | | | 23 (protein) |
| rpoC | RNA polymerase subunit β′ | 2.7.7.6 | b3988 | 24 (DNA) |
| | | | | 25 (protein) |
| spoT | bifunctional (p)ppGpp | 3.1.7.2 | b3650 | 26 (DNA) |
| | synthase/hydrolase SpoT | | | 27 (protein) |
| nusG | transcription termination factor | N/A | b3982 | 28 (DNA) |
| | NusG | | | 29 (protein) |
| flu | CP4-44 prophage; self | N/A | b2000 | 30 (DNA) |
| | recognizing antigen 43 (Ag43) | | | 31 (protein) |
| | autotransporter | | | |
| Ion | DNA-binding, ATP-dependent | 3.4.21.53 | b0439 | 32 (DNA) |
| | protease La | | | 33 (protein) |
| ygaH | L-valine exporter - YgaH subunit | N/A | b2683 | 34 (DNA) |
| | | | | 35 (protein) |
| rpsA | 30S ribosomal subunit protein | N/A | b0911 | 36 (DNA) |
| | S1 | | | 37 (protein) |

TABLE 2B
Homologs or orthologs identified by protein BLAST (BLASTP) of E. coli K-12 MG1655 proteins against protein databases from
selected reference organisms. Hits with the largest e-value are shown, and hits are only shown when the e-value < 1.0.

| | | | | | Ralstonia | Corynebacterium |
|---|---|---|---|---|---|---|
| Protein | B. subtilis | P. putida | L. plantarum | L. lactis | eutropha | glutamicum |
| (# aa) | 168 | KT2440 | JDM1 | KF147 | H16 | ATCC 13032 |
| MetJ | | | 38% identity (50 aa) | | | 26% identity (99 aa) |
| (105 aa) | | | “histidine | | | “hypothetical |
| | | | protein kinase; | | | protein NCgl2236” |
| | | | sensor protein” | | | (NP_601518.1) |
| | | | (YP_003063223.1) | | | |
| RelA | 39% identity | 32-48% identity | 37% identity | 38% identity | 32-42% identity | 37% identity |
| (744 aa) | (694 aa) “GTP | (681-751 aa) | (732 aa) “GTP | (697 aa) “GTP | (684-717 aa) | (688 aa) |
| | pyrophosphokinase” | “(p)ppGpp | pyrophosphokinase” | pyrophosphokinase/ | “GTP | “guanosine |
| | (NP_390638.2) | synthetase I | (YP_003063260.1) | guanosine-3,5-bis(di- | pyrophosphokinase” | polyphosphate pyro- |
| | | SpoT/RelA” | | phosphate) 3-pyro- | (YP_725845.1, | phosphohydrolase/ |
| | | (NP_743813.1, | | phosphohydrolase” | YP_725468.1) | synthetase” |
| | | NP_747403.1) | | (YP_003352549.1) | | (NP_600866.1) |
| PurT | 57% identity | 69% identity | 22-26% identity | 24% identity | 59% identity | 57% identity |
| (392 aa) | (378 aa) | (392 aa) | (329-363 aa) | (351 aa) | (382 aa) | (396 aa) |
| | “phospho- | “phospho- | “phospho- | “phospho- | “phospho- | “phospho- |
| | ribosylglycinamide | ribosylglycinamide | ribosylamino- | ribosylamino- | ribosylglycinamide | ribosylglycinamide |
| | formyltransferase” | formyltransfrase 2” | imidazole | imidazole | formyltransferase 2” | formyltransferase 2” |
| | (NP_388105.1) | (NP_743615.1) | carboxylase | carboxylase | (YP_726361.1) | (NP_601954.1) |
| | | | ATPase subunit” | NCAIR mutase | | |
| | | | (YP_003063771.1, | subunit” | | |
| | | | YP_003062522.1) | (YP_003354057.1) | | |
| AcrB | 24% identity | 54-66% identity | 25% identity (88 aa) | 25% identity (72 aa) | 26-61% identity | 31% identity (64 aa) |
| (1049 aa) | (806 aa) | (1016-1039 aa) | “prenyltransferase” | “hypothetical | (1033-1066 aa) | “short chain |
| | “surfactin | “hydrophobe/ | (YP_003062880.1) | protein LLKF_0319” | “cation/multidrug | dehydrogenase” |
| | self-resistance | amphiphile | | (YP_003352791.1), | efflux pump” | (NP_601672.1) |
| | transporter” | efflux (HAE1) | | 30% identity (63 aa) | (YP_728154.1, | |
| | (NP_388553.1) | family transporter” | | “multidrug | YP_728030.1, | |
| | | (NP_743544.1, | | resistance ABC | YP_727793.1, | |
| | | NP_745594.1) | | transporter ATP- | YP_726250.1, | |
| | | | | binding/permease” | YP_726630.1, | |
| | | | | (YP_003353188.1) | YP_725100.1, | |
| | | | | | YP_727320.1) | |
| FabR | 29% identity (80 aa) | 38% identity | 28% identity (96 aa) | 24-31% identity | 23-31% identity | 23-33% identity |
| (234 aa) | “HTH-type | (203 aa) | “transcription | (98-136 aa) | (97-147 aa) | (48-110 aa) |
| | transcriptional | “TetR family | regulator” | “TetR family | “TetR/AcrR family | “transcriptional |
| | regulator YxbF” | transcriptional | (YP_003063005.1), | transcriptional | transcriptional | regulator” |
| | (NP_391864.1) | regulator” | 25% identity | regulator” | regulator” | (NP_600383.1, |
| | | (NP_746964.1) | (223 aa) | (YP_003354897.1, | (YP_726724.1, | NP_601305.1, |
| | | | “transcription | YP_003354066.1) | YP_728152.1, | NP_600189.1) |
| | | | regulator” | | YP_727318.1); | |
| | | | (YP_003061828.1) | | 29% identity | |
| | | | | | (142 aa) | |
| | | | | | “response | |
| | | | | | regulator” | |
| | | | | | (YP_724722.1) | |
| YfgF | 26% identity | 26-28% identity | 23% identity | 23% identity | 28% identity | 33% identity |
| (747 aa) | (434 aa) | (467-484 aa) | (155-231 aa) | (241 aa) | (439 aa) | (258 aa) |
| | “diguanylate | “sensory box | “diguanylate | “cyclic di- | “sensor protein” | “diguanylate |
| | cyclase” | protein/GGDEF | cyclase/ | GMP-specific | (YP_725212.1) | cyclase” |
| | (YP_054577.1) | family protein” | phosphodiesterase | phosphodiesterase” | | (NP_600263.1) |
| | | (NP_743917.1), | domain-containing | (YP_003353123.1) | | |
| | | “sensory | protein” | | | |
| | | box protein” | (YP_003062219.1, | | | |
| | | (NP_742833.1) | YP_003063933.1) | | | |
| RzpD | 23% identity (98 aa) | 25% identity | 29% identity (51 aa) | 37% identity (35 aa) | | 35% identity (37 aa) |
| (153 aa) | “ferrous iron | (126 aa) | “hypothetical protein | “metal ABC | | “3-oxoacyl- |
| | permease EfeU” | “DNA-directed | JDM1_0327” | transporter substrate- | | ACP synthase” |
| | (NP_391707.2) | RNA polymerase | (YP_003061913.1), | binding protein” | | (NP_601696.1) |
| | | subunit beta” | 28% identity (90 aa) | (YP_003353799.1) | | |
| | | (NP_742613.1) | “SLT domain | | | |
| | | | protein” | | | |
| | | | (YP_003062580.1) | | | |
| YhjA | 42% identity (36 aa) | 43% identity | 32% identity (57 aa) | | 26% identity | 37% identity (41 aa) |
| (465 aa) | “menaquinol- | (294 aa) | “hypothetical | | (114 aa) “C-type | “glycosyl- |
| | cytochrome c | “cytochrome c551 | protein JDM1_1287” | | cytochrome (SoxD)” | transferase” |
| | reductase b/c | peroxidase” | (YP_003062871.1) | | (YP_727997.1) | (NP_600879.1) |
| | subunit” | (NP_745087.1) | | | | |
| | (NP_390135.1) | | | | | |
| GtrS | | | 30% identity (77 aa) | | | 33% identity (36 aa) |
| (443 aa) | | | “hypothetical | | | “hypothetical |
| | | | protein JDM1_2362” | | | protein NCgl1728” |
| | | | (YP_003063946.1) | | | (NP_601005.1) |
| YcdU | | 46% identity (33 aa) | | | 33% identity (33 aa) | 26% identity (46 aa) |
| (328 aa) | | “hydrophobe/ | | | “response | “hypothetical |
| | | amphiphile efflux-1 | | | regulator” | protein NCgl0613” |
| | | (HAE1) family | | | (YP_727560.1) | (NP_599874.1) |
| | | transporter” | | | | |
| | | (NP_745564.1) | | | | |
| IscR | 32% identity | 65% identity | 34% identity (70 aa) | 24% identity | 54% identity | 30-35% identity |
| (162 aa) | (131 aa) | (142 aa) “BadM/ | “transcription | (140 aa) | (136 aa) | (44-101 aa) |
| | “Rrf2 family | Rrf2 family | regulator” | “Rrf2 family | “transcriptional | “transcriptional |
| | transcriptional | transcriptional | (YP_003062417.1) | transcriptional | regulator of iron | regulator” |
| | regulator” | regulator” | | regulator” | sulfur cluster | (NP_600099.1, |
| | (NP_390630.2); | (NP_743002.1) | | (YP_003353004.1) | assembly (IscR)” | NP_601856.1, |
| | 29% identity | | | | (YP_725666.1) | NP_600589.1) |
| | (136 aa) | | | | | |
| | “HTH-type | | | | | |
| | transcriptional | | | | | |
| | regulator YwgB” | | | | | |
| | (NP_391638.1); | | | | | |
| | 31% identity | | | | | |
| | (137 aa) | | | | | |
| | “Rrf2 family | | | | | |
| | transcriptional | | | | | |
| | regulator” | | | | | |
| | (NP_388819.1) | | | | | |
| SspA | | 57% identity | 45% identity (22 aa) | | 46% identity | 56% identity (16 aa) |
| (212 aa) | | (200 aa) | “hypothetical | | (203 aa) | “hypothetical |
| | | “stringent | protein JDM1_0823” | | “stringent | protein NCgl2333” |
| | | starvation | (YP_003062407.1) | | starvation | (NP_601617.1) |
| | | protein A” | | | protein A” | |
| | | (NP_743480.1) | | | (YP_727831.1) | |
| Rph | 58% identity | 69% identity | | 26% identity | 62% identity | 59% identity |
| (228 aa) | (222 aa) | (228 aa) | | (207 aa) | (221 aa) | (217 aa) |
| | “ribonuclease | “ribonuclease | | “polyribonucleotide | “ribonuclease | “ribonuclease |
| | PH” | PH” | | nucleotidyl- | PH” | PH” |
| | (NP_390715.1) | (NP_747395.1) | | transferase” | (YP_725462.1) | (NP_601703.2) |
| PyrE | 25-34% identity | 67% identity | 29% identity | 24-30% identity | 56% identity | 29% identity |
| (213 aa) | (in stretches) | (213 aa) | (138 aa) | (in stretches) | (215 aa) | (139 aa) |
| | “orotate | “orotate | “orotate | “orotate | “orotate | “orotate |
| | phosphoribosyl- | phosphoribosyl- | phosphoribosyl- | phosphoribosyl- | phosphoribosyl- | phosphoribosyl- |
| | transferase” | transferase” | transferase” | transferase” | transferase” | transferase” |
| | (NP_389439.1) | (NP_747392.1) | (YP_003063746.1) | (YP_003354448.1) | (YP_724744.1) | (NP_601967.1) |
| NanK | 30% identity | 32% identity (65 aa) | 25-28% identity | 28% identity | 41% identity (37 aa) | 27% identity |
| (291 aa) | (310 aa) | “DNA gyrase | (256-296 aa) | (313 aa) | “ATP-dependent | (319 aa) |
| | “glucokinase” | subunit A” | “sugar kinase and | “glucokinase” | helicase” | “glucose kinase” |
| | (NP_390365.2) | (NP_743923.1), | transcription | (YP_003354608.1), | (YP_725935.1) | (NP_601389.1) |
| | | 27% identity (75 aa) | regulator” | 29% identity | | |
| | | “D,D-heptose | (YP_003063958.1, | (293 aa) “ROK | | |
| | | 1,7-bisphosphate | YP_003061957.1, | family glucokinase/ | | |
| | | phosphatase” | YP_003064483.1, | transcription | | |
| | | (NP_742229.1) | YP_003063668.1, | regulator” | | |
| | | | YP_003062678.1, | (YP_003354028.1) | | |
| | | | YP_003064434.1), | | | |
| | | | 27% identity | | | |
| | | | (313 aa) | | | |
| | | | “glucokinase” | | | |
| | | | (YP_003062902.1) | | | |
| RpoA | 46% identity | 74% identity | 47% identity | 41% identity | 61% identity | 45% identity |
| (329 aa) | (312 aa) | (326 aa) | (311 aa) | (319 aa) | (323 aa) | (315 aa) |
| | “DNA-directed | “DNA-directed | “DNA-directed | “DNA-directed | “DNA-directed | “DNA-directed |
| | RNA polymerase | RNA polymerase | RNA polymerase | RNA polymerase | RNA polymerase | RNA polymerase |
| | subunit alpha” | subunit alpha” | subunit alpha” | subunit alpha” | subunit alpha” | subunit alpha” |
| | (NP_388024.1) | (NP_742645.1) | (YP_003062460.1) | (YP_003354712.1) | (YP_727894.1) | (NP_599801.1) |
| RpoB | 47-59% identity | 72% identity | 47-52% identity | 46-47% identity | 66% identity | 42.56% identity |
| (1342 aa) | (in stretches) | (1360 aa) | (in stretches) | (in stretches) | (1370 aa) | (in stretches) |
| | “DNA-directed | “DNA-directed | “DNA-directed | “DNA-directed | “DNA-directed | “DNA-directed |
| | RNA polymerase | RNA polymerase | RNA polymerase | RNA polymerase | RNA polymerase | RNA polymerase |
| | subunit beta” | subunit beta” | subunit beta” | subunit beta” | subunit beta” | subunit beta” |
| | (NP_387988.2) | (NP_742613.1) | (YP_003062426.1) | (YP_003354373.1) | (YP_727933.1) | (NP_599733.1) |
| RpoC | 50% identity | 75% identity | 44-51% identity | 48-52% identity | 67% identity | 46-50% identity |
| (1407 aa) | (in stretches) | (1399 aa) | (in stretches) | (in stretches) | (1397 aa) | (in stretches) |
| | “DNA-directed | “DNA-directed | “DNA-directed | “DNA-directed | “DNA-directed | “DNA-directed |
| | RNA polymerase | RNA polymerase | RNA polymerase | RNA polymerase | RNA polymerase | RNA polymerase |
| | subunit beta′” | subunit beta′” | subunit beta′” | subunit beta′” | subunit beta′” | subunit beta′” |
| | (NP_387989.2) | (NP_742614.1) | (YP_003062427.1) | (YP_003354372.1) | (YP_727932.1) | (NP_599734.1) |
| SpoT | 40% identity | 37-55% identity | 38% identity | 40% identity | 36-47% identity | 38% identity |
| (702 aa) | (719 aa) “GTP | (681-701 aa) | (741 aa) “GTP | (725 aa) “GTP | (674-720 aa) “GTP | (723 aa) |
| | pyrophosphokinase” | “(p)ppGpp | pyrophosphokinase” | pyrophosphokinase/ | pyrophosphokinase” | “guanosine |
| | (NP_390638.2) | synthetase I | (YP_003063260.1) | guanosine-3,5- | (YP_725468.1, | polyphosphate |
| | | SpoT/RelA” | | bis(diphosphate) 3- | YP_725845.1) | pyrophosphohydrolase/ |
| | | (NP_747403.1, | | pyrophosphohydrolase | | synthetase” |
| | | NP_743813.1) | | (YP_003352549.1) | | (NP_600866.1) |
| NusG | 44% identity | 73% identity | 42% identity | 38% identity | 64% identity | 37% identity |
| (181 aa) | (177 aa) | (174 aa) | (183 aa) | (173 aa) | (179 aa) | (194 aa) |
| | “transcription | “transcription | “transcription | “transcription | “transcription | “transcription |
| | termination/ | antitermination | antitermination | antitermination | antitermination | antitermination |
| | antitermination | protein NusG” | protein NusG” | protein NusG” | factor NusG” | factor NusG” |
| | protein NusG” | (NP_742608.1) | (YP_003062140.1) | (YP_003354744.1) | (YP_727938.1) | (NP_599720.1) |
| | (NP_387982.1) | | | | | |
| Flu | | 25-29% identity | | 25% identity (83 aa) | | 31% identity (83 aa) |
| (1039 aa) | | (414-484 aa) | | “gluconate | | “carboxylesterase |
| | | “outer membrane | | permease” | | type B” |
| | | autotransporter” | | (YP_003354819.1) | | (NP_600361.2) |
| | | (NP_745213.1, | | | | |
| | | NP_744035.1) | | | | |
| Lon | 56% identity | 41-70% identity | 29% identity | 31% identity | 70% identity | 25% identity |
| (784 aa) | (765 aa) “Lon | (757-763 aa) | (104 aa) | (103 aa) | (773 aa) | (114 aa) |
| | protease 1” | “ATP-dependent | “endopeptidase | “hypothetical | “ATP-dependent | “ATPase |
| | (NP_390698.1) | protease La” | La” | protein | Lon protease” | with chaperone |
| | | (NP_744451.1, | (YP_003063372.1) | LLKF_2407” | (YP_725987.1) | activity” |
| | | NP_743601.1) | | (YP_003354798.1) | | (NP_601235.2) |
| YgaH | | | 31% identity | 25% identity | | |
| (111 aa) | | | (101 aa) | (56 aa) “peptide | | |
| | | | “hypothetical | deformylase” | | |
| | | | protein JDM1_1611” | (YP_003353006.1) | | |
| | | | (YP_003063195.1), | | | |
| | | | 37% identity (38 aa) | | | |
| | | | “hypothetical | | | |
| | | | protein JDM1_0741” | | | |
| | | | (YP_003062325.1) | | | |
| RpsA | 29-39% identity | 74% identity | 32-37% identity | 29-41% identity | 67% identity | 31-46% identity |
| (557 aa) | (in stretches) | (554 aa) | (in stretches) | (in stretches) | (529 aa) | (in stretches) |
| | “30S ribosomal | “30S ribosomal | “30S ribosomal | “30S ribosomal | “30S ribosomal | “30S ribosomal |
| | protein S1 | protein S1” | protein S1” | protein S1” | protein S1” | protein S1” |
| | homolog” | (NP_743928.2) | (YP_003063166.1) | (YP_003353306.1) | (YP_725313.1) | (NP_600575.1) |
| | (NP_390169.1) | | | | | |

So, in one aspect, there is provided a bacterial cell according to any one of the preceding aspects and embodiments, wherein each recited gene is instead (i) a gene encoding the corresponding (homolog or ortholog) protein in Table 2A or 2B above, (ii) a gene located at the corresponding locus, or (iii) both.

In particular, without being limited to theory, improved tolerance toward an aliphatic diol or other aliphatic polyol can be achieved by one or more genetic modifications which increase one or more of (a) the biosynthesis of methionine in the bacterial cell; (b) growth of the bacterial cell during polyol-induced methionine starvation, and (c) reduced efflux of precursors or intermediates required for methionine biosynthesis. This can, e.g., be achieved by a reduced expression of metJ, optionally also of relA and purT, and/or one or more other genetic modifications described herein.

In one embodiment, the bacterial cell has a genetic modification which reduces the expression of one or more endogenous proteins selected from the group consisting of
A transcriptional repressor of a methionine regulon
A murein endopeptidase
A cytochrome C peroxidase
A DNA-binding transcriptional dual regulator
A stringent starvation protein, synthesized predominantly when cells are exposed to amino acid starvation
An ribonuclease PH
A GDP pyrophosphokinase/GTP pyrophosphokinase
A phosphoribosylglycinamide formyltransferase
A permease subunit of a multidrug efflux system
A DNA-binding transcriptional repressor
A cyclic di-GMP phosphodiesterase.

### Example 1

Methods

Screening for Tolerance in Wild-Type Cells

Escherichia coli K-12 MG1655 was grown overnight in M9 minimal medium+1% glucose and subcultured the following morning to an initial OD₆₀₀ of 0.05 in M9+1% glucose. Cells were grown to mid-exponential phase (OD₅₀₀ 0.7-1.0) and were back-diluted with fresh medium to an OD₆₀₀ of 0.7. The diluted cells were used to inoculate M9+1% glucose containing varying concentrations of diols, and growth was measured in FlowerPlates in a Biolector microbioreactor system (m2p-labs) at 37° C. with 1000 rpm shaking. The culture volume in each well was 1.4 mL.

Adaptive Laboratory Evolution of Tolerant Strains

Based on the screening results, existence of biological production routes, and application potential of different diols, two diols were selected for evolutions: 2,3-butanediol and 1,2-propanediol. E. coli K-12 MG1655 was grown overnight in M9 minimal medium and 150 μL was transferred the next day into 8 tubes containing 15 mL of M9+1% glucose+5% (v/v) 2,3-butanediol or 1,2-propanediol on a Tecan Evo robotic platform custom-designed for performing adaptive laboratory evolutions (ALE). Cells were cultured on a 37° C. heat block with stirring by magnetic stir bars. Culture OD₆₀₀ was monitored at times determined by a predictive custom script, and when the OD₆₀₀ reached approximately 0.3, 150 μL of culture was inoculated into a new tube with the same media concentration. Instrument downtime would occasionally result in cells overgrowing to saturation or an OD₆₀₀ greater than 0.3, and reinoculations were occasionally performed from cryogenic stocks of the population. When the growth rate was observed to substantially increase, the media concentration was changed. These concentration changes were to 5.5%, 6.5%, 7%, and 8% for 1,2-propanediol and to 6.5%, and 8% for 2,3-butanediol. Approximately 100 μL of each 7%, population (8 per chemical) were plated on LB agar and incubated at 37° C. overnight.

Primary Screening of ALE Isolates

Five colonies from wild-type K-12 MG1655 and 10 individual colonies deriving from each population were inoculated into 300 μL M9+1% glucose in 96 well deepwell plates and incubated in a 300 rpm plate shaker at 37° C. The next day, cells were diluted 10× in M9+1% glucose and 30 μL was transferred into clear-bottomed 96 well half-deepwell plates (with rectangular wells) containing M9+1% glucose and M9+1% glucose+8.89% (v/v) 2,3-butanediol or 1,2-propanediol, such that the final concentration of diol was 8% (v/v). In addition, cryogenic glycerol stocks of the overnight culture were saved in a 96 well plate format. Half deepwell plates were incubated at 37° C. with 225 rpm shaking in a Growth Profiler (Enzyscreen), with optical scans of the plates taken at 15 minute intervals. Green pixel values integrated over a 1 mm diameter circular area in each well were converted to OD₅₀₀ values using a previously determined calibration between OD₅₀₀ and green pixel values. Resulting growth curves were visually inspected for isolates exhibiting the most robust or unique growth patterns within each population. In general, it was attempted to select three isolates per population for further analysis, and all populations were represented in the resequenced isolates.

Secondary Screening of ALE Isolates

Selected isolates from the primary screen were restruck onto LB agar from the cryogenic stock made from the overnight culture plate for the primary screen. Five K-12 MG1655 colonies and three individual colonies from each isolate were inoculated as biological replicates into a new 96 well deepwell plate containing 300 μL of M9+1% glucose, and grown overnight as for the primary screen. The next day, a cryogenic stock and half deepwell plates containing M9+1% glucose with or without diols were inoculated using the plate of overnight cultures, and growth was measured as described for the primary screen. Resulting growth curves were visually inspected for isolates exhibiting robust and reproducible growth between replicates in high concentrations of diols.

Re-Sequencing of ALE Isolates

A total of 20 isolates were selected from the secondary screen for whole-genome resequencing. An individual colony was taken from the LB agar plates prepared following the primary screen, inoculated into 2 mL LB, and grown overnight at 37° C. in a 250 rpm shaker. The following morning, 0.5 mL of cells were transferred to microcentrifuge tubes and centrifuged at 16000×g for 2 minutes. The supernatant was removed and pellets were stored at −20° C. until further processing. Genomic DNA was extracted from thawed cell pellets using a PureLink genomic DNA extraction kit, with further concentration and purification performed by ethanol precipitation. To generate libraries for sequencing, the Illumina TruSeq Nano kit was used according to the manufacturers' directions using an input quantity of 200 ng of genomic DNA from each isolate. Sequencing was performed on an Illumina MiSeq sequencer, with a minimum 20× average genomic coverage ensured for each isolate based on the number of reads. Fastq output files were analyzed for variants compared to the K-12 MG1655 reference genome (accession number NC_000913.3) using breseq.

Construction of Gene Knockouts

Probable important losses-of-function were determined by identifying genes across all isolates that harbored mutations, especially those occurring in multiple populations, and by the presence of at least one mutation that either generated a premature stop codon, a frameshift mutation, or the presence of an insertion element sequence within the gene. For those genes, the corresponding knockout strain from the Keio collection of single knockout mutants (where each gene is replaced with a cassette consisting of a kanamycin resistance gene flanked by FRT sites) was used as a donor strain for Plvir phage transduction (Baba et al., 2006). Briefly, the Keio strain was grown to early exponential phase in LB+5 mM CaCl₂ and 80 μL of a Plvir stock raised on K-12 MG1655 was added. After significant lysis was observed after 1.5 to 2 hours, the lysate was filter-sterilized to remove cells and stored at 4° C. Strain K-12 MG1655 was grown overnight in LB+5 mM CaCl₂ and 100 μL of the overnight culture was mixed with 100 μL of the Plvir lysate of the Keio collection mutant, and the mixture was incubated at 37° C. without shaking for 20 minutes. The entire mixture was then plated on LB agar containing 1.25 mM sodium pyrophosphate as a chelating agent and 25 μg/mL kanamycin. One colony was then restruck on LB+1.25 mM Na₂P₄O₇+25 μg/mL kanamycin plate and analyzed for presence of the Keio cassette in place of the wild-type gene by colony PCR. When further knockouts were constructed in the same strain, the Keio cassette was flipped out to generate a scar sequence such that Kan^{R} marker could be recycled. This was performed by transforming with pCP20, which constitutively expresses a flippase recombinase, and plating cells on LB agar+100 μg/mL ampicillin and incubating at 30° C. The next day, one or more colonies was tested by colony PCR for loss of the Keio cassette, and successful mutants were then cured of pCP20 by elevated temperature curing at 40° C. Strains were verified to be cured of plasmid by plating on LB agar+100 μg/mL ampicillin and incubation at 30° C. Plvir transductions were then performed using these mutant strains as recipients.

Biolector Growth Screening of Evolved Isolates and Reconstructed Mutants

Biological triplicate cultures of each strain were grown to saturation overnight in 96 well deepwell plates containing 300 μL M9+1% glucose. The next day, cells were diluted 1:10 in deionized water in a clear 96 well plate and the OD₅₀₀ was measured on a BioTek plate reader. 48 well FlowerPlates containing a final volume of 1.4 mL of M9+1% glucose+8% (v/v) 1,2-propanediol or 7% (v/v) 2,3-butanediol were inoculated to OD₅₀₀ 0.03 (with plate reader pathlength, 200 μL volume) with the overnight culture and sealed with Breathseal film. Light backscatter intensity was monitored in a Biolector microbioreactor system at 37° C. with 1000 rpm shaking. The Biolector screening concentration of 2,3-butanediol had to be reduced to 7% from 8% due to lack of growth at the higher concentration. Oxygen transfer rates are lower in the Biolector than in the Growth Profiler screening setup, resulting in reduced aeration of cultures.

Keio Collection Screening for Loss-of-Function Mutations

For primary screening, Keio collection mutants were inoculated directly from a cryogenic stock of the Keio collection into 300 μL LB medium containing 25 μg/mL kanamycin in 96 well deepwell plates and grown at 37° C. with 300 rpm shaking overnight. The Keio background strain, BW25113, was also inoculated into wells of this plate as a control. A cryogenic stock was made from each plate, and the cryogenic stock was replica plated into another 96 well deepwell plate containing 300 μL M9+1% glucose and grown overnight. The next day, cells were inoculated 1:100 into clear bottomed 96 well half-deepwell plates containing M9+1% glucose plus 6% and 7% 2,3-butanediol or 6% and 8% 1,2-propanediol, and cultivated in a Growth Profiler as previously described for screening of ALE isolates.

As a secondary screen, promising Keio collection mutants were struck on LB+25 μg/mL kanamycin from the cryogenic stock plate prepared during primary screening above and biological triplicate colonies were inoculated into a 96 well deepwell plate containing 300 μL M9+1% glucose. The next day, cells were inoculated into plates for cultivation on the Growth Profiler as described above.

Methionine Supplementation

Cultures of selected strains/isolates were grown as described above for Biolector growth screening of evolved isolates and reconstructed mutants. L-methionine was supplemented to the media to a final concentration of 0.3 g/L. Generation of 2,3-butanediol production strains

The Keio collection strain containing hsdR::kan, JW4313, was used as the donor strain for Plvir phage transduction into recipient strains K-12 MG1655 and all 23BD evolved isolates as described in ‘Construction of gene knockouts’. Plasmid pCP20, which encodes a constitutively expressed yeast flippase recombinase (FLP), was transformed into each P1 transduced strain to remove the kanamycin resistance marker, generating the equivalent ΔhsdR strains.

Plasmid pET-RABC was obtained from Dr. Cuiqing Ma and Dr. Chao Gao (Shandong University; Xu et al., 2014). The hsdR deletion was found to be necessary for transformation in K-12 MG1655 due to the presence of EcoKI (HsdM/HsdR/HsdS) restriction sites in the plasmid. The plasmid was transformed into each ΔhsdR strain by adding the plasmid to cells resuspended in TSS buffer followed by heat shocking for 30 seconds at 42° C., placing the cells on ice, resuspending in LB, and outgrowing at 37° C. for 1-2 hours. The outgrown cells were plated on LB agar plates containing 50 μg/mL kanamycin to select for transformants.

2,3-Butanediol Production Run

Individual colonies of 2,3-butanediol production strains were picked as biological replicates and inoculated into 300 μL of M9 medium containing 5% (w/v) glucose, 1% (w/v) yeast extract, and 50 μg/mL kanamycin in 96-well deepwell plates with metal sandwich covers. Plates were grown overnight in a plate shaker at 37° C. with 300 rpm shaking. The next morning, 22 μL of cells were inoculated into 2 mL of M9 medium containing 5% (w/v) glucose, 50 μg/mL kanamycin, and 1% (w/v) yeast extract in 24-well deepwell plates, and grown in a plate shaker at 30° C. and 300 rpm shaking. After 48 hours, culture supernatants were collected.

HPLC Analysis of 2,3-Butanediol

Culture supernatants were injected (30 μL) onto an Aminex HPX-87H ion exclusion column held at 30° C. on a Dionex UltiMate HPLC system equipped with a Shodex RI-101 refractive index detector held at 45° C. The mobile phase was 5 mM sulfuric acid and was kept at a constant flow rate of 0.6 mL/min. 2,3-butanediol (from a standard composed of a mixture of racemic and meso forms) was found to elute as two overlapping peaks. Concentrations were calculated using a standard calibration curve (linear response with R²=0.9999) and adding up the areas of both peaks.

Cross-Compound Tolerance Screening

96 well deepwell plates containing 300 μL of M9+1% glucose were inoculated directly from cryogenic stocks made from precultures for the secondary screening of ALE isolates and were grown overnight at 37° C. with 300 rpm shaking. The next day, cells were diluted 1:100 into 96 well half-deepwell plates containing the following final concentrations of each chemical in M9+1% glucose:

| | | |
|---|---|---|
| Butanol | 1.4% | v/v |
| Glutarate | 40 | g/L |
| p-coumarate | 7.5 | g/L |
| Putrescine | 32 | g/L |
| HMDA | 32 | g/L |
| Adipate | 45 | g/L |
| Isobutyrate | 7.5 | g/L |
| Hexanoate | 3 | g/L |
| Octanoate | 8 | g/L |
| 2,3-butanediol | 6% | v/v |
| 1,2-propanediol | 6% | v/v |

| | |
|---|---|
| sodium chloride | 0.6M |

Plates were cultivated in a Growth Profiler for 48 hours as described for screening of ALE isolates. Green pixel integrated values from each well were converted to OD₆₀₀ values using a calibration curve and the resulting OD₆₀₀ vs. elapsed time data was processed using custom scripts to determine the time required for each culture to reach an OD of 1.0 (t_{OD1}). This value is a combined measure of growth rate and lag time in each culture. The median value was taken for biological triplicates of each isolate and was normalized to the median t_{OD1} for K-12 MG1655 controls (5 replicates). The ratio of t_{OD1(evolved)}/t_{OD1(wild-type)} is presented.

The same cultivation method as described above was also used to determine growth parameters (growth rate and lag time) in different defined concentrations of other diols, as described in the next section.

Analysis of Growth Parameters (Growth Rate and Lag Time)

For data obtained with the Biolector microbioreactor system, self-baselined growth series were imported directly into a custom software platform that automatically detects growth phases and exports growth rates and lag times. In this software, a line was fit to a detected linear region in semilog space to determine the growth rate.

For data obtained with the Growth Profiler, an algorithm was implemented that automatically detected the pixel integration region in each well in each image by locating the darkest pixels in each well. These values were converted to OD₆₀₀ with a calibration run in the same manner. Growth parameters were automatically determined as described for Biolector data above, but with a newer version of the software that implemented a direct exponential fit of a detected growth phase in linear space. Additionally, the software implemented an adaptive smoothing algorithm that split the data into variable sized windows that minimize the standard deviation of growth values within a time interval, and generated spline fits between points. Finally, the software discarded regions where growth curves were fit but the signal-to-noise ratio was less than 1, to eliminate automatic detection of false growth phases. While automatic detection succeeded in detecting and fitting the dominant growth phase more than 95% of the time, all data was additionally manually curated to ensure that the main growth phase was always selected and that false growth phases were not detected when growth was essentially absent.

Results

Wild-Type Tolerance to Diols

E. coli K-12 MG1655 exhibited a steadily decreasing growth rate as a function of diol concentration in general (Table 3). Toxicity appeared to depend on carbon chain length, with toxicity increasing in order of 1,2-propanediol, 2,3-butanediol, and the pentanediols. Toxicity was much greater for 1,2-pentanediol than for 1,5-pentanediol, with growth observed at maximum concentrations of 1% and 3.5%, respectively. Maximum concentrations for robust growth in 2,3-butanediol and 1,2-propanediol were 5% and 7.5%, respectively.

TABLE 3
Growth of K-12 MG1655 in varying concentrations
of different diols (neutralized).

| | 2,3-butanediol | | | | 1,2-propanediol | | | |
|---|---|---|---|---|---|---|---|---|
| | Mean | | std. error | | mean | | std. error | |
| diol % | μ | t_{lag} | μ | t_{lag} | μ | t_{lag} | μ | t_{lag} |
| (v/v) | (h⁻¹) | (h) | (h⁻¹) | (h) | (h⁻¹) | (h) | (h⁻¹) | (h) |
| 0 | 0.988 | 2.3 | 0.079 | 0.2 | 0.701 | 0.9 | 0.019 | 0.1 |
| 0.5 | 0.944 | 2.2 | 0.144 | 0.6 | 0.701 | 0.9 | 0.042 | 0.2 |
| 1 | 0.791 | 1.8 | 0.058 | 0.2 | 0.662 | 0.8 | 0.022 | 0.2 |
| 2 | 0.754 | 2.1 | 0.065 | 0.4 | 0.595 | 0.8 | 0.021 | 0.4 |
| 3.5 | 0.498 | 1.6 | 0.060 | 0.8 | 0.442 | 0.5 | 0.024 | 0.4 |
| 5 | 0.265 | −1.2 | 0.017 | 0.8 | 0.406 | 2.3 | 0.014 | 0.3 |
| 7.5 | 0.054 | — | 0.094 | — | 0.579 | 8.3 | 0.034 | 0.2 |
| 10 | 0.000 | — | 0.000 | — | 0.000 | — | 0.000 | — |

| | 1,5-pentanediol | | | | 1,2-pentanediol | | | |
|---|---|---|---|---|---|---|---|---|
| | mean | | std. error | | mean | | std. error | |
| diol % | μ | t_{lag} | μ | t_{lag} | μ | t_{lag} | μ | t_{lag} |
| (v/v) | (h⁻¹) | (h) | (h⁻¹) | (h) | (h⁻¹) | (h) | (h⁻¹) | (h) |
| 0 | 0.699 | 0.8 | 0.009 | 0.0 | 0.701 | 0.9 | 0.019 | 0.1 |
| 0.5 | 0.613 | 0.3 | 0.003 | 0.1 | 0.524 | 0.4 | 0.025 | 0.3 |
| 1 | 0.569 | 0.5 | 0.013 | 0.2 | 0.346 | 1.3 | 0.032 | 0.8 |
| 2 | 0.437 | 0.9 | 0.003 | 0.1 | 0.000 | — | 0.000 | — |
| 3.5 | 0.158 | −8.8 | 0.075 | 15.1 | 0.000 | — | 0.000 | — |
| 5 | 0.000 | — | 0.000 | — | 0.000 | — | 0.000 | — |
| 7.5 | 0.000 | — | 0.000 | — | 0.000 | — | 0.000 | — |
| 10 | 0.000 | — | 0.000 | — | 0.000 | — | 0.000 | — |

Based on these results and aiming for an initial growth rate of approximately 0.3-0.4 h⁻¹, it was decided to begin evolutions at a concentration of 5% (v/v) for both 2,3-butanediol and 1,2-propanediol.

Resequencing of Tolerant Isolates

Variants detected in 2,3-butanediol and 1,2-propanediol evolved isolates are presented in Tables 4 and 5. Each strain name corresponds to the chemical the strain was isolated from, the population the strain was isolated from, and the original number of the strain assigned during primary screening (e.g. 23BD1-6 is an 2,3-butanediol-evolved strain isolated from population 1). In each table, strains are arranged such that all that were isolated from the same population are presented in the same rows. Strains with an asterisk (*) following their name are hypermutator strains, and only the mutation identified that can be associated with generating the hypermutator phenotype (mutations in mutS, mutY, or mutL) and those mutations that are shared with other mutations in the same gene in other strains are shown. For the 1,2-propanediol populations, the majority of isolates were hypermutator strains, with the exception of 12PD4-6, 12PD6-3, and 12PD6-9. A large number of called missing coverage deletions in 12PD6-9 were likely a result of an adapter problem, and these are not considered. For mutator strains, only mutations in genes (or surrounding intergenic regions) that were common between the mutator isolates and the non-mutator isolates are listed.

Mutations that occur independently across multiple populations, or that appear fixed in a highly variable population, are likely causative and of highest interest. For 2,3-butanediol, these include mutations in metJ, relA, nanK, purT, rpoB, and rpoC. Mutations also occur in acrB in 2 populations. Of these mutations, those of metJ, relA, purT, and acrB are likely loss-of-function mutations, due to the presence of frameshift mutations, large deletions, or IS element insertions in at least one population of individual isolate that possesses mutations in that gene. Other mutations are likely gain-of-function or weakening of function, for example coding mutations in genes encoding subunits of RNA polymerase (RpoB and RpoC), which are essential, and the T128S mutation in NanK, which is present in nearly every population.

For 1,2-propanediol, mutations in metJ were all coding, however they are also assumed to be loss-of-function mutations due to the co-occurrence of probable loss-of-function mutations for 2,3-butanediol. Because most isolates were hypermutators, SNPs are expected to be more common than other types of mutations. There were additionally mutations in relA in most isolates, which are also presumed to be losses-of-function based on loss-of-function mutations found for 2,3-butanediol evolved isolates. Mutations in fabR and yfgF co-occurred in population 12PD6 and both were presumed to be losses-of-function due to an intergenic IS element insertion upstream of fabR in 12PD7-5, and an IS element insertion and large deletion in yfgG in 12PD6-3 (a non-mutator strain) and 12PD8-7, respectively. Mutations also occurred in c/sA across multiple mutator 12PD populations (not shown in Table 3) that appeared to have different lineages based on the mutation in the mutator gene, with 12PD3-10 having a premature stop codon in that gene (W428*).

TABLE 4
Variants detected in 2,3-butanediol-evolved isolates

| coordinate | gene | change | coordinate | gene | change | coordinate | gene | change |
|---|---|---|---|---|---|---|---|---|
| 23BD1-6 | | | 23BD1-9 | | | | | |
| 998193 | elfA | IS5 element | 998193 | elfA | IS5 element | | | |
| | | insertion | | | insertion | | | |
| 1347480 | rnb | IS5 element | 1347480 | rnb | IS5 element | | | |
| | | insertion | | | insertion | | | |
| 1931977 | purT | V366G (T→G) | 1931977 | purT | V366G (T→G) | | | |
| 2794550 | gabP | W100G (T→G) | 2794550 | gabP | W100G (T→G) | | | |
| 2911491 | [relA][gudD] | 7528 bp deletion | 2911491 | [relA][gudD] | 7528 bp deletion | | | |
| 3369969 | nanK | T128S (T→A) | 3369969 | nanK | T128S (T→A) | | | |
| 4128380 | metJ | G6C (C→A) | 3762316 | rhsA | 2905 bp deletion | | | |
| 4182583 | rpoB | H447Y (C→T) | 4128380 | metJ | G6C (C→A) | | | |
| | | | 4182583 | rpoB | H447Y (C→T) | | | |
| 23BD2-4 | | | 23BD2-7 | | | 23BD2-9 | | |
| 580116 | ybcW/ylcI | IS5 element | 580116 | ybcW/ylcI | IS5 element | 580116 | ybcW/ylcI | IS5 element |
| | | insertion | | | insertion | | | insertion |
| 962056 | rpsA | G21V (G→T) | 962056 | rpsA | G21V (G→T) | 962056 | rpsA | G21V (G→T) |
| 1979639 | insA/uspC | noncoding | 1096841 | ycdU/serX | IS2 element | 2911491 | [relA][gudD] | 7528 bp deletion |
| | | SNP (T→C) | | | insertion | | | |
| 2470411 | gtrS | noncoding | 2911491 | [relA][gudD] | 7528 bp deletion | 3369969 | nanK | T128S (T→A) |
| | | SNP (A→G) | | | | | | |
| 2911491 | [relA][gudD] | 7528 bp deletion | 3369969 | nanK | T128S (T→A) | 4128293 | metJ | IS5 element |
| | | | | | | | | insertion |
| 3369969 | nanK | T128S (T→A) | 4128293 | metJ | IS5 element | 4186152 | rpoC | L268R (T→G) |
| | | | | | insertion | | | |
| 4128293 | metJ | IS5 element | 4186152 | rpoC | L268R (T→G) | | | |
| | | insertion | | | | | | |
| 4186152 | rpoC | L268R (T→G) | | | | | | |
| 23BD3-3* | | | 23BD3-4* | | | 23BD3-9* | | |
| 483726 | acrB | 1 bp deletion | 483726 | acrB | 1 bp deletion | 483726 | acrB | 1 bp deletion |
| 2911491 | [relA][gudD] | 7528 bp deletion | 2911491 | [relA][gudD] | 7528 bp deletion | 2795142 | gabP | V297A (T→C) |
| 3369969 | nanK | T128S (T→A) | 3369969 | nanK | T128S (T→A) | 2911491 | [relA][gudD] | 7528 bp deletion |
| 4128316 | metJ | V27A (A→G) | 4128316 | metJ | V27A (A→G) | 3369969 | nanK | T128S (T→A) |
| 4182890 | rpoB | D549G (A→G) | 4182890 | rpoB | D549G (A→G) | 4128316 | metJ | V27A (A→G) |
| 4184036 | rpoB | V931A (T→C) | 4184036 | rpoB | V931A (T→C) | 4182890 | rpoB | D549G (A→G) |
| 4184514 | rpoB | noncoding | 4398051 | mutL | 7 bp deletion | 4184036 | rpoB | V931A (T→C) |
| | | SNP (C→T) | | | | | | |
| 4398051 | mutL | 7 bp deletion | | | | 4398051 | mutL | 7 bp deletion |
| 23BD4-3 | | | 23BD4-4 | | | 23BD4-7 | | |
| 1230727 | hlyE/umuD | noncoding | 1230727 | hlyE/umuD | noncoding | 2911491 | [relA][gudD] | 7528 bp deletion |
| | | SNP (C→A) | | | SNP (C→A) | | | |
| 1347775 | rnb | E380* (C→A) | 1879829 | yeaR | IS186 element | 3369969 | nanK | T128S (T→A) |
| | | | | | insertion | | | |
| 1879829 | yeaR | IS186 element | 1931977 | purT | V366G (T→G) | 4128169 | metJ | D76A (T→G) |
| | | insertion | | | | | | |
| 1931977 | purT | V366G (T→G) | 2913536 | relA | W39* (C→T) | 4186274 | rpoC | N309Y (A→T) |
| 2810756 | ygaH/mprA | noncoding | 4128361 | metJ | Y12C (T→C) | | | |
| | | SNP (G→T) | | | | | | |
| 2913536 | relA | W39* (C→T) | 4187815 | rpoC | 15 bp deletion | | | |
| 4031019 | fadB/pepQ | noncoding | | | | | | |
| | | SNP (G→A) | | | | | | |
| 4128361 | metJ | Y12C (T→C) | | | | | | |
| 4187815 | rpoC | 15 bp deletion | | | | | | |
| 23BD5-1 | | | 23BD5-7 | | | 23BD5-10 | | |
| 1230727 | hlyE/umuD | noncoding | 679090 | ybeT | L106P (A→G) | 2911491 | [relA][gudD] | 7528 bp deletion |
| | | SNP (C→A) | | | | | | |
| 1879829 | yeaR | IS186 element | 824028 | ybhP | L157P (A→G) | 3369969 | nanK | T128S (T→A) |
| | | insertion | | | | | | |
| 1931977 | purT | V366G (T→G) | 1722386 | ydhK | T89M (C→T) | 4128169 | metJ | D76A (T→G) |
| 2913536 | relA | W39* (C→T) | 2911491 | [relA][gudD] | 7528 bp deletion | 4186274 | rpoC | N309Y (A→T) |
| 3823036 | spoT | I213L (A→C) | 3369969 | nanK | T128S (T→A) | | | |
| 4128361 | metJ | Y12C (T→C) | 3438773 | zntR | S120R (T→G) | | | |
| 4187815 | rpoC | 15 bp deletion | 4128379 | metJ | G6D (C→T) | | | |
| | | | 4186274 | rpoC | N309Y (A→T) | | | |
| 23BD6-1 | | | | | | | | |
| 575786 | [nmpC][borD] | 3027 bp deletion | | | | | | |
| 1930993 | purT | V38G (T→G) | | | | | | |
| 2912611 | relA | 10 bp deletion | | | | | | |
| 4128250 | metJ | L49R (A→C) | | | | | | |
| 4178172 | nusG | F144V (T→G) | | | | | | |
| 4185573 | rpoC | Y75C (A→G) | | | | | | |
| 23BD7-4 | | | 23BD7-5 | | | 23BD7-7 | | |
| 998719 | elfD | IS2 element | 484098 | acrB | 2 bp insertion | 998719 | elfD | IS2 element |
| | | insertion | | | (→AT) | | | insertion |
| 1931668 | purT | L263W (T→G) | 998719 | elfD | IS2 element | 1413735 | rcbA | Y2* (A→T) |
| | | | | | insertion | | | |
| 2073463 | flu | L642Q (T→A) | 1931499 | purT | IS5 element | 1931668 | purT | L263W (T→G) |
| | | | | | insertion | | | |
| 2911491 | [relA][gudD] | 7528 bp deletion | 2073463 | flu | L642Q (T→A) | 2073463 | flu | L642Q (T→A) |
| 3178128 | tolC | L5R (T→G) | 2911491 | [relA][gudD] | 7528 bp deletion | 2911491 | [relA][gudD] | 7528 bp deletion |
| 3369969 | nanK | T128S (T→A) | 3369969 | nanK | T128S (T→A) | 3178128 | tolC | L5R (T→G) |
| 3668878 | yhjA | IS2 element | 4128386 | metJ | W4G (A→C) | 3369969 | nanK | T128S (T→A) |
| | | insertion | | | | | | |
| 4128386 | metJ | W4G (A→C) | 4186152 | rpoC | L268R (T→G) | 3668878 | yhjA | IS2 element |
| | | | | | | | | insertion |
| 4186152 | rpoC | L268R (T→G) | | | | 4128386 | metJ | W4G (A→C) |
| 4466841 | treR | IS5 element | | | | 4186152 | rpoC | L268R (T→G) |
| | | insertion | | | | | | |
| | | | | | | 4466841 | treR | IS5 element |
| | | | | | | | | insertion |
| 23BD8-2 | | | 23BD8-7 | | | | | |
| 461034 | lon | I716S (T→G) | 365741 | lacZ | 1 bp deletion | | | |
| 2661816 | iscR | T106P (T→G) | 2661816 | iscR | T106P (T→G) | | | |
| 2810459 | ygaH | V39A (T→C) | 2810459 | ygaH | V39A (T→C) | | | |
| 2913641 | relA | V4E (A→T) | 2913641 | relA | V4E (A→T) | | | |
| 3815809 | pyrE/rph | 1 bp deletion | 3815809 | pyrE/rph | 1 bp deletion | | | |
| 4184579 | rpoB | I1112S (T→G) | 4128212 | metJ | F62L (A→G) | | | |
| | | | 4184579 | rpoB | I1112S (T→G) | | | |

TABLE 5
Variants detected in 1,2-propanediol-evolved isolates.

| coordinate | gene | change | coordinate | gene | change | coordinate | gene | change |
|---|---|---|---|---|---|---|---|---|
| 12PD1-2* | | | 12PD1-4* | | | 12PD1-10* | | |
| 2406631 | IrhA | A4V (G→A) | 2406631 | IrhA | A4V (G→A) | 2780330 | ypjA | noncoding |
| | | | | | | | | SNP (G→A) |
| 2780330 | ypjA | noncoding | 2780330 | ypjA | noncoding | 2912885 | relA | I256T (A→G) |
| | | SNP (G→A) | | | SNP (G→A) | | | |
| 2912885 | relA | I256T (A→G) | 2912885 | relA | I256T (A→G) | 3377215 | sspA | L69P (A→G) |
| 3377215 | sspA | L69P (A→G) | 3377215 | sspA | L69P (A→G) | 3440116 | rpoA | D305G (T→C) |
| 3440116 | rpoA | D305G (T→C) | 3440116 | rpoA | D305G (T→C) | 4128316 | metJ | V27A (A→G) |
| 3815801 | pyrE/rph | 1 bp deletion | 3815801 | pyrE/rph | 1 bp deletion | 3815801 | pyrE/rph | 1 bp deletion |
| 4128316 | metJ | V27A (A→G) | 4128316 | metJ | V27A (A→G) | 4399151 | mutL | 1 bp deletion |
| 4399151 | mutL | 1 bp deletion | 4399151 | mutL | 1 bp deletion | | | |
| 12PD2-8* | | | 12PD2-9* | | | | | |
| 2780330 | ypjA | noncoding | 2780330 | ypjA | noncoding | | | |
| | | SNP (G→A) | | | SNP (G→A) | | | |
| 2912885 | relA | I256T (A→G) | 2912885 | relA | I256T (A→G) | | | |
| 3377215 | sspA | L69P (A→G) | 3377215 | sspA | L69P (A→G) | | | |
| 3440116 | rpoA | D305G (T→C) | 3440116 | rpoA | D305G (T→C) | | | |
| 3815801 | pyrE/rph | 1 bp deletion | 3815801 | pyrE/rph | 1 bp deletion | | | |
| 4128316 | metJ | V27A (A→G) | 4128316 | metJ | V27A (A→G) | | | |
| 4399151 | mutL | 1 bp deletion | 4399151 | mutL | 1 bp deletion | | | |
| 12PD3-7* | | | 12PD3-8* | | | 12PD3-10* | | |
| 2780330 | ypjA | noncoding | 2780330 | ypjA | noncoding | 2912357 | relA | H432R (T→C) |
| | | SNP (G→A) | | | SNP (G→A) | | | |
| 2912885 | relA | I256T (A→G) | 2912885 | relA | I256T (A→G) | 3377215 | sspA | L69P (A→G) |
| 3377215 | sspA | L69P (A→G) | 3377215 | sspA | L69P (A→G) | 3377215 | sspA | L69P (A→G) |
| 3440116 | rpoA | D305G (T→C) | 3440116 | rpoA | D305G (T→C) | 4128247 | metJ | R50H (C→T) |
| 3815801 | pyrE/rph | 1 bp deletion | 3815801 | pyrE/rph | 1 bp deletion | 3815801 | pyrE/rph | 1 bp deletion |
| 4128316 | metJ | V27A (A→G) | 4128316 | metJ | V27A (A→G) | 4399151 | mutL | 1 bp deletion |
| 4399151 | mutL | 1 bp deletion | 4399151 | mutL | 1 bp deletion | | | |
| 12PD4-6 | | | 12PD4-8* | | | 12PD4-9* | | |
| 962923 | rpsA | D310V (A→T) | 1879829 | yeaR | IS186 element | 2405949 | IrhA | noncoding |
| | | | | | insertion | | | SNP (G→A) |
| 1879829 | yeaR | IS186 element | 2858929 | mutS | T613P (A→C) | 2858929 | mutS | T613P (A→C) |
| | | insertion | | | | | | |
| 2912634 | relA | T340P (T→G) | 2913035 | relA | L206P (A→G) | 2913035 | relA | L206P (A→G) |
| 3377240 | sspA | T61P (T→G) | 2913196 | relA | noncoding | 2913196 | relA | noncoding |
| | | | | | SNP (T→C) | | | SNP (T→C) |
| 4128078 | metJ | *106Y (T→G) | 3815801 | pyrE/rph | 1 bp deletion | 3815801 | pyrE/rph | 1 bp deletion |
| | | | 4128197 | metJ | T67A (T→C) | 4128197 | metJ | T67A (T→C) |
| 12PD5-1* | | | | | | | | |
| 2780291 | ypjA | noncoding | 2780291 | ypjA | noncoding | | | |
| | | SNP (G→A) | | | SNP (G→A) | | | |
| 3377215 | sspA | L69P (A→G) | 3377215 | sspA | L69P (A→G) | | | |
| 3815801 | pyrE/rph | 1 bp deletion | 3815801 | pyrE/rph | 1 bp deletion | | | |
| 4128316 | metJ | V27A (A→G) | 4128316 | metJ | V27A (A→G) | | | |
| 4161466 | fabR | G115S (G→A) | 4161466 | fabR | G115S (G→A) | | | |
| 4399151 | mutL | 1 bp deletion | 4399151 | mutL | 1 bp deletion | | | |
| 12PD6-3 | | | 12PD6-9 | | | | | |
| 2406831 | IrhA/alaA | IS2 element | 2628621 | yfgF | 62 bp deletion | | | |
| | | insertion | | | | | | |
| 2628616 | yfgF | IS2 element | 2780609 | ypjA | noncoding | | | |
| | | insertion | | | SNP (A→C) | | | |
| 3440194 | rpoA | G279V (C→A) | 2780609 | ypjA | noncoding | | | |
| | | | | | SNP (A→C) | | | |
| 4035240 | rrsA | 1768 bp deletion | 3440194 | rpoA | G279V (C→A) | | | |
| 4161155 | fabR | T11N (C→A) | 4161155 | fabR | T11N (C→A) | | | |
| 4208083 | [symbol] sE/gltV/rrlE/rr [symbol] | 5021 bp deletion | | | | | | |
| 4261586 | yjbM/dusA | noncoding | | | | | | |
| | | SNP (A→C) | | | | | | |
| 12PD7-5* | | | 12PD7-6* | | | | | |
| 2913035 | relA | L206P (A→G) | 1879829 | yeaR | IS186 element | | | |
| | | | | | insertion | | | |
| 2913196 | relA | noncoding | 2913035 | relA | L206P (A→G) | | | |
| | | SNP (T→C) | | | | | | |
| 3815801 | pyrE/rph | 1 bp deletion | 2913196 | relA | noncoding | | | |
| | | | | | SNP (T→C) | | | |
| 4128316 | metJ | V27A (A→G) | 3815801 | pyrE/rph | 1 bp deletion | | | |
| 4160984 | sthA/fabR | IS2 element | 3815521 | pyrE | V83A (A→G) | | | |
| | | insertion | | | | | | |
| 4161391 | fabR | T90A (A→G) | 4128247 | metJ | R50H (C→T) | | | |
| 12PD8-6* | | | 12PD8-7* | | | 12PD8-10* | | |
| 2912885 | relA | I256T (A→G) | 2406923 | IrhA/alaA | noncoding | 2912885 | relA | I256T (A→G) |
| | | | | | SNP (T→C) | | | |
| 3376927 | sspA | L165P (A→G) | 2628850 | yfgF | 409 bp deletion | 3376927 | sspA | L165P (A→G) |
| 3815801 | pyrE/rph | 1 bp deletion | 2912885 | relA | I256T (A→G) | 3815801 | pyrE/rph | 1 bp deletion |
| 3816407 | rph | noncoding | 3376927 | sspA | L165P (A→G) | 3816407 | rph | noncoding |
| | | SNP (G→A) | | | | | | SNP (G→A) |
| 4128197 | metJ | T67A (T→C) | 3815801 | pyrE/rph | 1 bp deletion | 4128197 | metJ | T67A (T→C) |
| | | | 3816407 | rph | noncoding | | | |
| | | | | | SNP (G→A) | | | |
| | | | 4128197 | metJ | T67A (T→C) | | | |

[symbol]

indicates data missing or illegible when filed

Characterization of Selected Isolates

Each re-sequenced isolate was characterized using the Biolector system for growth in M9 media containing 7% (v/v) 2,3-butanediol or 8% (v/v) 1,2-propanediol in biological triplicates. Tables showing the calculated average growth rates and lag times for each isolate of each detected phase (using custom automated growth parameter determination software) are shown in Table 6 for 2,3-butanediol, and Table 7 for 1,2-propanediol. Standard errors are standard deviations about the mean of the growth rate and lag time for the three independent biological replicates. In the presence of diols, many strains exhibited diauxic or triauxic growth patterns, manifesting in the presence of multiple growth phases. A value is only shown for second and third phases if two or more replicates had a growth phase detected, and that value is the average of the parameters calculated for those determined growth phases.

Large differences in growth behavior amongst evolved isolates can be noted. Better growing strains are defined by both the slope of the curve (higher growth rate) and at what time the cultures begin growing (reduced lag time). Wild-type K-12 MG1655 did not grow in 7% 2,3-butanediol within 48 hours in 2 out of 3 biological replicates (the remaining biological replicate had a growth rate of 0.32 h⁻¹ with a 28.3 h lag time). All other isolates grew robustly but with a variety of lag times.

TABLE 6
Growth rates and lag times of re-sequenced 2,3-butanediol
evolved isolates in M9 + 7% (v/v) 2,3-butanediol.

| | phase 1 | | | | phase 2 | | | |
|---|---|---|---|---|---|---|---|---|
| | mean | | std. error | | Mean | | std. error | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | — | — | — | — | — | — | — | — |
| 23BD1-6 | 0.258 | 4.4 | 0.037 | 0.7 | 0.544 | 13.8 | 0.109 | 0.4 |
| 23BD1-9 | 0.297 | 5.2 | 0.033 | 1.9 | 0.547 | 13.9 | 0.063 | 0.3 |
| 23BD2-4 | 0.464 | 6.5 | 0.014 | 0.5 | 0.515 | 15.0 | 0.139 | 3.6 |
| 23BD2-7 | 0.543 | 7.7 | 0.025 | 0.4 | 0.409 | 14.2 | 0.023 | 0.4 |
| 23BD2-9 | 0.476 | 14.0 | 0.091 | 0.7 | 0.326 | 19.4 | 0.036 | 11.4 |
| 23BD3-3 | 0.430 | 12.9 | 0.062 | 2.7 | 0.371 | 21.1 | 0.119 | 13.8 |
| 23BD3-4 | 0.471 | 6.8 | 0.081 | 1.4 | — | — | — | — |
| 23BD3-9 | 0.379 | 12.2 | 0.069 | 1.2 | — | — | — | — |
| 23BD4-3 | 0.386 | 9.4 | 0.105 | 0.6 | 0.495 | 14.3 | 0.041 | 0.9 |
| 23BD4-4 | 0.393 | 21.4 | 0.043 | 0.7 | 0.554 | 37.2 | 0.176 | 1.3 |
| 23BD4-7 | 0.411 | 17.3 | 0.072 | 1.1 | 0.592 | 24.6 | 0.042 | 11.7 |
| 23BD5-1 | 0.597 | 8.9 | 0.054 | 1.8 | 0.944 | 16.7 | 0.209 | 0.4 |
| 23BD5-7 | 0.244 | 4.2 | 0.049 | 0.7 | 0.667 | 17.0 | 0.180 | 0.6 |
| 23BD5-10 | 0.389 | 9.0 | 0.050 | 0.3 | 0.473 | 13.5 | 0.202 | 5.5 |
| 23BD6-1 | 0.351 | 6.0 | 0.014 | 1.2 | — | — | — | — |
| 23BD7-4 | 0.471 | 4.9 | 0.016 | 0.4 | — | — | — | — |
| 23BD7-5 | 0.475 | 5.3 | 0.051 | 0.7 | 0.687 | 14.3 | 0.258 | 1.5 |
| 23BD7-7 | 0.379 | 2.9 | 0.037 | 0.8 | — | — | — | — |
| 23BD8-2 | 0.417 | 12.2 | 0.058 | 1.5 | 0.285 | 10.0 | 0.249 | 6.1 |
| 23BD8-7 | 0.507 | 6.1 | 0.051 | 1.4 | — | — | — | — |

| phase 3 | |
|---|---|
| Mean | std. error |

| | μ | t_{lag} | μ | t_{lag} |
|---|---|---|---|---|
| Strain | (h⁻¹) | (h) | (h⁻¹) | (h) |
| MG1655 | — | — | — | — |
| 23BD1-6 | — | — | — | — |
| 23BD1-9 | — | — | — | — |
| 23BD2-4 | 0.396 | 12.5 | 0.325 | 8.3 |
| 23BD2-7 | 0.603 | 19.6 | 0.119 | 1.2 |
| 23BD2-9 | 0.678 | 20.1 | 0.135 | 0.9 |
| 23BD3-3 | 0.478 | 18.2 | — | — |
| 23BD3-4 | — | — | — | — |
| 23BD3-9 | — | — | — | — |
| 23BD4-3 | — | — | — | — |
| 23BD4-4 | — | — | — | — |
| 23BD4-7 | — | — | — | — |
| 23BD5-1 | — | — | — | — |
| 23BD5-7 | — | — | — | — |
| 23BD5-10 | 0.382 | 12.6 | 0.386 | 4.9 |
| 23BD6-1 | — | — | — | — |
| 23BD7-4 | — | — | — | — |
| 23BD7-5 | — | — | — | — |
| 23BD7-7 | — | — | — | — |
| 23BD8-2 | — | — | — | — |
| 23BD8-7 | — | — | — | — |

TABLE 7
Growth rates and lag times of re-sequenced 1,2-propanediol
evolved isolates in M9 + 8% (v/v) 1,2-propanediol.

| | phase 1 | | | | phase 2 | | | |
|---|---|---|---|---|---|---|---|---|
| | Mean | | std. error | | mean | | std. error | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.352 | 6.2 | 0.078 | 1.5 | 0.336 | 10.9 | 0.071 | 1.1 |
| 12PD1-2 | 0.703 | 5.0 | 0.169 | 1.8 | — | — | — | — |
| 12PD1-4 | 0.692 | 6.1 | 0.085 | 5.1 | 1.069 | 5.6 | 0.069 | 0.5 |
| 12PD1-10 | 0.754 | 3.0 | 0.030 | 0.2 | — | — | — | — |
| 12PD2-8 | 0.598 | 9.3 | 0.138 | 5.0 | — | — | — | — |
| 12PD2-9 | 0.328 | 5.8 | 0.005 | 1.1 | — | — | — | — |
| 12PD3-7 | 0.584 | 6.7 | 0.039 | 1.5 | — | — | — | — |
| 12PD3-8 | 0.588 | 3.9 | 0.060 | 1.2 | 0.801 | 11.6 | 0.268 | 1.7 |
| 12PD3-10 | 0.532 | 3.9 | 0.054 | 0.6 | 0.468 | 9.7 | 0.175 | 1.2 |
| 12PD4-6 | 0.749 | 6.7 | 0.209 | 3.8 | — | — | — | — |
| 12PD4-8 | 0.566 | 3.5 | 0.073 | 0.3 | — | — | — | — |
| 12PD4-9 | 0.677 | 4.2 | 0.062 | 0.3 | — | — | — | — |
| 12PD5-1 | 0.668 | 1.9 | 0.013 | 0.1 | — | — | — | — |
| 12PD5-3 | 0.499 | 1.5 | 0.150 | 4.5 | — | — | — | — |
| 12PD6-3 | 0.432 | 0.5 | 0.023 | 0.5 | 0.351 | 2.9 | 0.329 | 11.7 |
| 12PD6-9 | 0.546 | 1.3 | 0.146 | 0.4 | — | — | — | — |
| 12PD7-5 | 0.629 | 5.3 | 0.234 | 5.8 | — | — | — | — |
| 12PD7-6 | 0.737 | 2.7 | 0.060 | 0.6 | 0.376 | 8.0 | 0.106 | 1.4 |
| 12PD8-6 | 0.589 | 6.0 | 0.007 | 2.9 | — | — | — | — |
| 12PD8-7 | 0.502 | 5.0 | 0.025 | 2.5 | — | — | — | — |
| 12PD8-10 | 0.455 | 1.6 | 0.089 | 0.2 | — | — | — | — |

Knockout Strain Growth Performance

Probable loss-of-function mutations were identified from re-sequencing results as described in methods and the section on resequencing of selected isolates. Initially, single gene knockouts of metJ, relA, purT, fabR, dsA, yfgF, treA, and acrB were constructed and tested with a selection of evolved isolates in 7% (v/v) 2,3-butanediol (Table 8) or 8% (v/v) 1,2-propanediol (Table 9). The wild-type strain and the majority of single knockout strains did not grow in 7% 2,3-butanediol. Only the metJ knockout, and to a much lesser extent the purT knockout, exhibited detectable growth phases. For growth in 8% 1,2-propanediol, only the metJ knockout and the acrB knockout (with more variability) exhibited primary growth phases with higher growth rates than wild-type K-12 MG1655.

Because metJ losses-of-function always co-occurred with probable relA losses-of-function in nearly every resequenced evolved isolate, and most other apparent loss-of-function mutations co-occurred with other mutations, double knockouts were next tested and screened in the Biolector test format for co-occurring combinations (Table 10). For growth in 2,3-butanediol, the only double knockout with improved growth over K-12 MG1655 metJ::kan was K-12 MG1655 ΔmetJ acrB::kan. Other knockout combinations with metJ exhibited abolished growth relative to the metJ knockout alone.

The same double gene knockouts were also tested with 8% 1,2-propanediol (Table 11). In contrast to growth in 2,3-butanediol, K-12 ΔmetJ acrB::kan did not exhibit improved growth relative to the single knockout K-12 men:kan, nor did any other knockout combination with metJ. K-12 ΔfabR yfgF::kan exhibited an increased growth rate in the primary growth phase and reduced lag times relative to the fabR and yfgF single deletion strains, as well as an increased secondary growth phase (which was present but not automatically detected for at least 2 out of 3 replicates for the fabR and yfgF single deletion strains). It also had a reduced lag time relative to K-12 MG1655 and a higher secondary phase growth rate than K-12 MG1655.

Finally, triple gene deletions were also constructed and tested with 7% 2,3-butanediol (Table 12) and 8% 1,2-propanediol (Table 13), with the single knockout strain K-12 MG1655 acrB::kan also added. Additional genes were also tested in combination with deletions in metJ and relA, including mb (co-occurring mutations in population 23BD1 and 23BD4-3), treR (co-occurring mutations in 23BD7-4 and 23BD7-7), and yeaR (co-occurring mutations in 23BD4-3, 23BD4-4, and 23BD5-1).

TABLE 8
Growth rates and lag times of single gene knockouts in M9 +
7% (v/v) 2,3-butanediol as measured in the Biolector testing format.

| | phase 1 | | | | phase 2 | | | |
|---|---|---|---|---|---|---|---|---|
| | Mean | | std. error | | mean | | std. error | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | — | — | — | — | — | — | — | — |
| 23BD2-4 | 0.501 | 7.0 | 0.035 | 0.5 | 0.323 | 12.6 | 0.006 | 0.6 |
| 23BD4-3 | 0.341 | 6.5 | 0.027 | 0.8 | 0.589 | 15.2 | 0.027 | 0.2 |
| 23BD6-1 | 0.367 | 6.0 | 0.014 | 0.5 | — | — | — | — |
| 23BD7-4 | 0.478 | 4.7 | 0.024 | 0.3 | — | — | — | — |
| MG1655 metJ::kan | 0.162 | 4.9 | 0.017 | 2.4 | — | — | — | — |
| MG1655 relA::kan | — | — | — | — | — | — | — | — |
| MG1655 purT::kan | 0.093 | 11.5 | 0.017 | 3.6 | — | — | — | — |
| MG1655 fabR::kan | — | — | — | — | — | — | — | — |
| MG1655 clsA::kan | — | — | — | — | — | — | — | — |
| MG1655 yfgF::kan | — | — | — | — | — | — | — | — |
| MG1655 treA::kan | — | — | — | — | — | — | — | — |
| MG1655 acrB::kan | — | — | — | — | — | — | — | — |

TABLE 9
Growth rates and lag times of single gene knockouts in M9 +
8% (v/v) 1,2-propanediol as measured in the Biolector testing format.

| | phase 1 | | | | phase 2 | | | |
|---|---|---|---|---|---|---|---|---|
| | mean | | std. error | | mean | | std. error | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.271 | 8.5 | 0.030 | 4.0 | 0.279 | 11.6 | 0.018 | 0.5 |
| 12PD3-8 | 0.594 | 3.1 | 0.037 | 2.4 | 0.312 | 5.5 | 0.053 | 2.9 |
| 12PD4-6 | 0.531 | 14.6 | 0.012 | 3.7 | 0.309 | 12.4 | 0.290 | 8.3 |
| 12PD6-3 | 0.436 | 1.0 | 0.012 | 0.2 | 0.098 | −15.0 | 0.038 | 13.5 |
| 12PD7-6 | 0.834 | 4.1 | 0.010 | 0.2 | 0.543 | 10.9 | 0.112 | 1.0 |
| MG1655 metJ::kan | 0.346 | 4.1 | 0.030 | 0.7 | 0.667 | 13.6 | 0.079 | 0.5 |
| MG1655 relA::kan | 0.150 | 3.4 | 0.014 | 2.5 | — | — | — | — |
| MG1655 purT::kan | 0.245 | 3.9 | 0.054 | 1.9 | 0.264 | 1.3 | 0.256 | 12.1 |
| MG1655 fabR::kan | 0.133 | −3.4 | 0.036 | 10.1 | 0.452 | 13.3 | 0.185 | 3.3 |
| MG1655 clsA::kan | 0.186 | 2.9 | 0.039 | 1.6 | — | — | — | — |
| MG1655 yfgF::kan | 0.258 | 3.4 | 0.011 | 0.9 | 0.477 | 12.0 | 0.068 | 0.7 |
| MG1655 treA::kan | 0.295 | 5.0 | 0.083 | 2.9 | 0.420 | 12.4 | 0.066 | 1.3 |
| MG1655 acrB::kan | 0.369 | 4.9 | 0.196 | 1.2 | 0.373 | 11.1 | 0.159 | 1.7 |

TABLE 10
Growth rates and lag times of single and double gene knockouts in M9 +
7% (v/v) 2,3-butanediol as measured in the Biolector testing format.

| | phase 1 | | | | phase 2 | | | |
|---|---|---|---|---|---|---|---|---|
| | mean | | std. error | | mean | | std. error | |
| | μ | t_{lag} | μ | t_{lag} | μ | t_{lag} | μ | t_{lag} |
| Strain | (h⁻¹) | (h) | (h⁻¹) | (h) | (h⁻¹) | (h) | (h⁻¹) | (h) |
| MG1655 | — | — | — | — | — | — | — | — |
| 23BD2-4 | 0.396 | 6.2 | 0.026 | 0.7 | 0.465 | 14.4 | 0.127 | 1.4 |
| 23BD4-3 | 0.300 | 5.4 | 0.097 | 2.7 | 0.605 | 15.5 | 0.064 | 0.1 |
| 23BD6-1 | 0.264 | 9.6 | 0.005 | 1.3 | — | — | — | — |
| 23BD7-4 | 0.492 | 6.6 | 0.045 | 0.3 | — | — | — | — |
| MG1655 metJ::kan | * | * | * | * | — | — | — | — |
| MG1655 relA::kan | — | — | — | — | — | — | — | — |
| MG1655 purT::kan | — | — | — | — | — | — | — | — |
| MG1655 fabR::kan | — | — | — | — | — | — | — | — |
| MG1655 yfgF::kan | — | — | — | — | — | — | — | — |
| MG1655 ΔmetJ relA::kan | — | — | — | — | — | — | — | — |
| MG1655 ΔmetJ purT::kan | — | — | — | — | — | — | — | — |
| MG1655 ΔrelA purT::kan | — | — | — | — | — | — | — | — |
| MG1655 ΔmetJ acrB::kan | 0.254 | 9.5 | 0.035 | 0.2 | — | — | — | — |
| MG1655 ΔfabR yfgF::kan | — | — | — | — | — | — | — | — |

* growth was apparent but phase not detected in 2 out of 3 replicates

TABLE 11
Growth rates and lag times of single and double gene knockouts in M9 +
8% (v/v) 1,2-propanediol as measured in the Biolector testing format.

| | phase 1 | | | | phase 2 | | | |
|---|---|---|---|---|---|---|---|---|
| | mean | | std. error | | mean | | std. error | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.323 | 11.7 | 0.092 | 1.4 | 0.256 | 17.1 | 0.121 | 4.8 |
| 12PD3-8 | 0.556 | 7.9 | 0.020 | 2.7 | 0.314 | 11.9 | 0.018 | 3.1 |
| 12PD4-6 | 0.482 | 20.4 | 0.040 | 0.8 | 0.123 | 12.1 | 0.017 | 1.1 |
| 12PD6-3 | 0.399 | 0.2 | 0.025 | 0.8 | 0.128 | −1.0 | 0.022 | 5.4 |
| 12PD7-6 | 0.761 | 3.5 | 0.149 | 0.5 | 0.475 | 8.2 | 0.236 | 3.6 |
| MG1655 metJ::kan | 0.314 | 4.8 | 0.022 | 1.0 | 0.483 | 12.7 | 0.092 | 0.7 |
| MG1655 relA::kan | — | — | — | — | — | — | — | — |
| MG1655 purT::kan | 0.381 | 11.8 | 0.036 | 0.9 | — | — | — | — |
| MG1655 fabR::kan | 0.229 | 13.5 | 0.030 | 2.0 | — | — | — | — |
| MG1655 yfgF::kan | 0.260 | 12.5 | 0.039 | 2.3 | — | — | — | — |
| MG1655 ΔmetJ relA::kan | — | — | — | — | — | — | — | — |
| MG1655 ΔmetJ purT::kan | 0.275 | 7.3 | 0.070 | 5.8 | 0.304 | 10.3 | 0.002 | 0.1 |
| MG1655 ΔrelA purT::kan | 0.156 | 6.2 | 0.046 | 5.9 | — | — | — | — |
| MG1655 ΔmetJ acrB::kan | 0.196 | 5.2 | 0.016 | 0.9 | 0.221 | 9.8 | 0.053 | 3.5 |
| MG1655 ΔfabR yfgF::kan | 0.334 | 5.8 | 0.030 | 0.2 | 0.342 | 11.7 | 0.047 | 0.5 |

TABLE 12
Growth rates and lag times of selected single, double, and triple gene knockouts
in M9 + 7% (v/v) 2,3-butanediol as measured in the Biolector testing format.

| | phase 2 | | | | phase 3 | | | |
|---|---|---|---|---|---|---|---|---|
| | mean | | std. error | | Mean | | std. error | |
| | μ | t_{lag} | μ | t_{lag} | μ | t_{lag} | μ | t_{lag} |
| Strain | (h⁻¹) | (h) | (h⁻¹) | (h) | (h⁻¹) | (h) | (h⁻¹) | (h) |
| MG1655 | — | — | — | — | — | — | — | — |
| 23BD2-4 | 0.495 | 14.4 | 0.049 | 0.4 | 0.618 | 17.6 | 0.030 | 0.7 |
| 23BD4-3 | 0.831 | 14.3 | 0.070 | 0.2 | — | — | — | — |
| 23BD6-1 | — | — | — | — | — | — | — | — |
| 23BD7-4 | — | — | — | — | — | — | — | — |
| MG1655 metJ::kan | — | — | — | — | — | — | — | — |
| MG1655 acrB::kan | — | — | — | — | — | — | — | — |
| MG1655 ΔmetJ acrB::kan | — | — | — | — | — | — | — | — |
| MG1655 ΔmetJ ΔrelA acrB::kan | — | — | — | — | — | — | — | — |
| MG1655 ΔmetJ ΔrelA purT::kan | 0.244 | 12.0 | 0.006 | 0.5 | 0.395 | 31.4 | 0.036 | 0.2 |
| MG1655 ΔmetJ ΔrelA clsA::kan | — | — | — | — | — | — | — | — |
| MG1655 ΔmetJ ΔrelA rnb::kan | — | — | — | — | — | — | — | — |
| MG1655 ΔmetJ ΔrelA yeaR::kan | — | — | — | — | — | — | — | — |
| MG1655 ΔmetJ ΔrelA treR::kan | — | — | — | — | — | — | — | — |
| MG1655 ΔmetJ ΔrelA treA::kan | — | — | — | — | — | — | — | — |

| | phase 3 | | | |
|---|---|---|---|---|
| | Mean | | std. error | |
| | μ | t_{lag} | μ | t_{lag} |
| Strain | (h⁻¹) | (h) | (h⁻¹) | (h) |
| MG1655 | — | — | — | — |
| 23BD2-4 | 0.618 | 17.6 | 0.030 | 0.7 |
| 23BD4-3 | — | — | — | — |
| 23BD6-1 | — | — | — | — |
| 23BD7-4 | — | — | — | — |
| MG1655 metJ::kan | — | — | — | — |
| MG1655 acrB::kan | — | — | — | — |
| MG1655 ΔmetJ acrB::kan | — | — | — | — |
| MG1655 ΔmetJ ΔrelA acrB::kan | — | — | — | — |
| MG1655 ΔmetJ ΔrelA purT::kan | 0.395 | 31.4 | 0.036 | 0.2 |
| MG1655 ΔmetJ ΔrelA clsA::kan | — | — | — | — |
| MG1655 ΔmetJ ΔrelA rnb::kan | — | — | — | — |
| MG1655 ΔmetJ ΔrelA yeaR::kan | — | — | — | — |
| MG1655 ΔmetJ ΔrelA treR::kan | — | — | — | — |
| MG1655 ΔmetJ ΔrelA treA::kan | — | — | — | — |

For 2,3-butanediol, it was found that K-12 ΔmetJ ΔrelA purT::kan had an increased growth rate vs. K-12 men:kan, indicating a positive epistatis between these three loss-of-function mutations. The acrB single knockout strain did not have a detectable growth phase, also demonstrating that metJ and acrB losses-of-function have a synergetic effect when combined. None of the other triple knockout strains exhibited a detectable growth phase in at least 2 out of 3 replicates, with substantially lower growth than K-12 men:kan observed.

For 1,2-propanediol, K-12 Δmet.7 ΔrelA purT::kan had a higher average growth rate than K-12 metJ::kan alone although with higher variability (individual replicates had growth rates of 0.39, 0.64, and 0.32 h⁻¹). The K-12 ΔmetJ acrB::kan did not have an increased growth rate over K-12 metJ::kan alone (again), and no other triple knockout combination with metJ and relA exhibited a higher growth rate than K-12 metJ::kan.

The Keio collection of gene knockouts is a commercially available collection of knockouts in nearly all non-essential genes and ORFs in E. coli strain BW25113. This strain is a K-12 derivative and possesses known mutations relative to the K-12 MG1655 background. All Keio collection strains with knockouts in genes that were found to be mutated in Tables 4 and 5 were screened for growth against the BW25113 control in M9+1% glucose+6% (Table 14) or 7% (v/v) 2,3-butanediol, and 6% or 8% (v/v) 1,2-propanediol (Table 15). In 6% 2,3-butanediol, the yhjA, rzpD, ycdU, iscR, and gtrS knockout strains exhibited improved growth rates compared to the wild-type. In 7% 2,3-butanediol, growth was minimal and it was not possible to automatically calculate growth parameters for any strain except BW25113 rzpD::kan, which exhibited a growth rate of 0.065 h⁻¹. This strain also visually exhibited the strongest growth in this condition. Other strains which qualitatively had improved growth over K-12 MG1655 were the same as those with higher growth rates in 6% 2,3-butanediol, minus K-12 iscR::kan. Corresponding knockouts in K-12 MG1655 remain to be tested in the Biolector.

TABLE 13
Growth rates and lag times of selected single, double, and triple gene knockouts
in M9 + 8% (v/v) 1,2-propanediol as measured in the Biolector testing format.

| | phase 1 | | | | phase 2 | | | |
|---|---|---|---|---|---|---|---|---|
| | mean | | std. error | | mean | | std. error | |
| | μ | t_{lag} | μ | t_{lag} | μ | t_{lag} | μ | t_{lag} |
| Strain | (h⁻¹) | (h) | (h⁻¹) | (h) | (h⁻¹) | (h) | (h⁻¹) | (h) |
| MG1655 | 0.236 | 1.0 | 0.067 | 3.3 | 0.578 | 11.5 | 0.057 | 0.4 |
| 12PD3-8 | 0.661 | 1.4 | 0.130 | 0.5 | 0.389 | 3.3 | 0.242 | 4.9 |
| 12PD4-6 | 0.915 | 3.7 | 0.006 | 0.2 | 0.832 | 5.7 | 0.012 | 0.2 |
| 12PD6-3 | 0.409 | −0.8 | 0.055 | 1.4 | 0.127 | −0.2 | 0.016 | 3.6 |
| 12PD7-6 | 0.849 | 3.6 | 0.017 | 0.2 | 0.468 | 9.7 | 0.087 | 0.6 |
| MG1655 metJ::kan | 0.328 | 3.3 | 0.038 | 1.2 | 0.704 | 13.1 | 0.042 | 0.3 |
| MG1655 acrB::kan | 0.296 | 2.1 | 0.015 | 1.1 | 0.584 | 9.9 | 0.095 | 0.3 |
| MG1655 ΔmetJ acrB::kan | 0.342 | 4.0 | 0.005 | 0.8 | 0.631 | 13.2 | 0.084 | 0.4 |
| MG1655 ΔmetJ ΔrelA acrB::kan | 0.198 | 26.9 | 0.028 | 11.0 | — | — | — | — |
| MG1655 ΔmetJ ΔrelA purT::kan | 0.452 | 10.4 | 0.169 | 1.5 | — | — | — | — |
| MG1655 ΔmetJ ΔrelA clsA::kan | 0.259 | 7.0 | 0.081 | 3.6 | — | — | — | — |
| MG1655 ΔmetJ ΔrelA rnb::kan | 0.208 | 19.4 | 0.081 | 5.0 | — | — | — | — |
| MG1655 ΔmetJ ΔrelA yeaR::kan | 0.163 | 18.6 | 0.100 | 3.3 | — | — | — | — |
| MG1655 ΔmetJ ΔrelA treR::kan | 0.222 | 14.4 | 0.033 | 1.0 | — | — | — | — |
| MG1655 ΔmetJ ΔrelA treA::kan | 0.224 | 11.7 | 0.098 | 9.6 | — | — | — | — |

| phase 3 | |
|---|---|
| Mean | std. error |

| | μ | t_{lag} | μ | t_{lag} |
|---|---|---|---|---|
| Strain | (h⁻¹) | (h) | (h⁻¹) | (h) |
| MG1655 | — | — | — | — |
| 12PD3-8 | — | — | — | — |
| 12PD4-6 | 0.218 | 7.3 | 0.033 | 2.8 |
| 12PD6-3 | — | — | — | — |
| 12PD7-6 | — | — | — | — |
| MG1655 metJ::kan | 0.121 | 11.1 | 0.030 | 9.0 |
| MG1655 acrB::kan | — | — | — | — |
| MG1655 ΔmetJ acrB::kan | 0.125 | 15.5 | 0.005 | 1.1 |
| MG1655 ΔmetJ ΔrelA acrB::kan | — | — | — | — |
| MG1655 ΔmetJ ΔrelA purT::kan | — | — | — | — |
| MG1655 ΔmetJ ΔrelA clsA::kan | — | — | — | — |
| MG1655 ΔmetJ ΔrelA rnb::kan | — | — | — | — |
| MG1655 ΔmetJ ΔrelA yeaR::kan | — | — | — | — |
| MG1655 ΔmetJ ΔrelA treR::kan | — | — | — | — |
| MG1655 ΔmetJ ΔrelA treA::kan | — | — | — | — |

TABLE 14
Growth rates and lag times of Keio collection knockouts in M9 + 6%
(v/v) 2,3-butanediol as measured in the Growth Profiler testing format.
The growth of BW25113 nanK::kan was too low for automatic calculation.

| | |
|---|---|
| Mean | std. error |

| Strain | μ (h⁻¹) | t_{lag} (h) | μ (h⁻¹) | t_{lag} (h) |
|---|---|---|---|---|
| BW25113 | 0.191 | 6.1 | 0.014 | 0.5 |
| BW25113 fadB::kan | 0.225 | 5.6 | 0.031 | 0.6 |
| BW25113 ybhP::kan | 0.108 | 20.3 | 0.008 | 0.4 |
| BW25113 yhjA::kan | 0.295 | 8.0 | 0.025 | 1.9 |
| BW25113 ybeT::kan | 0.166 | 16.3 | 0.003 | 5.3 |
| BW25113 rhsA::kan | 0.169 | 10.5 | 0.029 | 3.2 |
| BW25113 rzpD::kan | 0.261 | 5.8 | 0.031 | 1.5 |
| BW25113 nanK::kan | ND | — | — | — |
| BW25113 ycdU::kan | 0.285 | 4.0 | 0.020 | 1.4 |
| BW25113 iscR::kan | 0.240 | 3.7 | 0.008 | 0.7 |
| BW25113 gtrS::kan | 0.250 | 2.0 | 0.007 | 2.4 |

For Keio mutants tested in 6% and 8% 1,2-propanediol (Table 13), only minor growth differences were observed, with the sspA knockout strain, and to a lesser extent the rph knockout strain (8% 1,2-propanediol only) exhibiting increased growth rates over wild-type BW25113. Corresponding knockouts in K-12 MG1655 are to be tested in the Biolector.

TABLE 15
Growth rates of Keio collection knockouts in M9 + 6% and 8% (v/v) 1,2-
propanediol as measured in the Growth Profiler testing format.

| | |
|---|---|
| 6% 1,2- | 8% 1,2- |
| propanediol | propanediol |

| | | std. | | std. |
|---|---|---|---|---|
| strain | μ (h⁻¹) | error | μ (h⁻¹) | error |
| BW25113 | 0.498 | 0.008 | 0.289 | 0.011 |
| BW25113 frdA::kan | 0.474 | 0.007 | 0.250 | 0.054 |
| BW25113 yfgF::kan | 0.405 | 0.055 | 0.173 | 0.021 |
| BW25113 ade::kan | 0.508 | 0.037 | 0.271 | 0.028 |
| BW25113 dusA::kan | 0.516 | 0.035 | 0.303 | 0.017 |
| BW25113 yagE::kan | 0.399 | 0.024 | 0.253 | 0.016 |
| BW25113 ecpC::kan | 0.378 | 0.037 | 0.138 | 0.029 |
| BW25113 yraQ::kan | 0.423 | 0.014 | 0.229 | 0.003 |
| BW25113 sspA::kan | 0.462 | 0.042 | 0.346 | 0.016 |
| BW25113 rph::kan | 0.438 | 0.030 | 0.313 | 0.008 |
| BW25113 ycdU::kan | 0.389 | 0.029 | 0.287 | 0.006 |
| BW25113 ypjA::kan | 0.381 | 0.001 | 0.260 | 0.007 |

Tabular summaries of knockout strains exhibiting improved growth in 2,3-butanediol and 1,2-propanediol as compared to the wild-type strain are shown in Tables 16 and 17

TABLE 16
Summary of knockout strains with improved growth over
the wild-type strain in 2,3-butanediol

| | Growth rate effect vs. | |
|---|---|---|
| | K-12 MG1655 or BW25113 | |
| | | 7% |
| Strain genotype | 6% 2,3-butanediol | 2,3-butanediol |
| K-12 MG1655 metJ::kan | not tested | moderate increase |
| K-12 MG1655 ΔmetJ acrB::kan | not tested | large increase |
| K-12 MG1655 ΔmetJ ΔrelA | not tested | large increase |
| purT::kan | | |
| BW25113 rzpD::kan | small increase | moderate increase |
| BW25113 yhjA::kan | small increase | small increase |
| BW25113 gtrS::kan | small increase | small increase |
| BW25113 ycdU::kan | small increase | small increase |
| BW25113 iscR::kan | small increase | None |

TABLE 17
Summary of knockout strains with improved growth over the
wild-type strain in 1,2-propanediol

| | Growth rate effect vs. K-12 MG1655 or | |
|---|---|---|
| | BW25113 | |
| Strain genotype | 6% 1,2-propanediol | 8% 1,2-propanediol |
| K-12 MG1655 metJ::kan | not tested | moderate increase |
| K-12 MG1655 ΔmetJ ΔrelA | not tested | large increase |
| purT::kan | | (variable) |
| K-12 MG1655 ΔfabR | not tested | moderate increase |
| yfgF::kan | | (secondary phase) |
| BW25113 sspA::kan | small increase | small increase |
| BW25113 rph::kan | None | small increase |

A few of the knockouts identified in the Keio collection screens were additionally constructed as mutants in K-12 MG1655 and tested in the Biolector format. Results for the rzpD and sspA knockouts grown in 7% v/v 2,3-butanediol are shown in Table 18, and results for the rph and sspA knockouts grown in 8% v/v 1,2-propanediol (first phase) are shown in Table 19. The sspA knockout exhibited a significantly increased growth rate and reduced lag time in 2,3-butanediol, however its improvement was less significant in 1,2-propanediol.

TABLE 18
Growth rates and lag times of additional single gene knockouts in
M9 + 7% (v/v) 2,3-butanediol as measured in the Biolector testing format.

| | |
|---|---|
| mean | std. error |

| strain | μ (h⁻¹) | t_{lag} (h) | μ (h⁻¹) | t_{lag} (h) |
|---|---|---|---|---|
| MG1655 | 0.019 | — | 0.017 | — |
| MG1655 rzpD::kan | 0.036 | 19.9 | 0.063 | — |
| MG1655 sspA::kan | 0.213 | 17.4 | 0.056 | 4.5 |

TABLE 19
Growth rates and lag times of additional single gene knockouts in
M9 + 8% (v/v) 1,2-propanediol as measured in the Biolector
testing format.

| | |
|---|---|
| mean | std. error |

| strain | μ (h⁻¹) | t_{lag} (h) | μ (h⁻¹) | t_{lag} (h) |
|---|---|---|---|---|
| MG1655 | 0.219 | 2.0 | 0.066 | 1.2 |
| MG1655 sspA::kan | 0.259 | 2.0 | 0.023 | 0.5 |
| MG1655 rph::kan | 0.249 | 1.3 | 0.050 | 2.2 |

Methionine Feeding Reveals Insights into Mechanisms

A strain evolved for high ethanol concentrations in the literature also exhibited a mutation in metJ, and it was shown that deletion of metJ or addition of excess methionine improved ethanol tolerance in wild-type cells (Haft et al., 2014). Without being limited to theory, as MetJ is a repressor controlling expression of several genes involved in methionine biosynthesis, a similar effect can occur with toxic concentrations of diols. The wild-type strain and a selection of evolved strains were first tested for growth with and without supplementation of the medium containing 6% (v/v) 2,3-butanediol with 0.3 g/L L-methionine (Table 20). Robust growth of K-12 MG1655 in 6% 2,3-butanediol was significantly restored by the addition of methionine, with a growth rate approaching that of evolved strains in 6% 2,3-butanediol. Evolved strains did not have a significantly enhanced growth rate increase in 2,3-butanediol with the addition of methionine.

Methionine supplementation was also tested for its ability to restore growth in the presence of 8% (v/v) 1,2-propanediol. Wild-type and a selection of evolved strains were tested (Table 21), and methionine was again found to restore growth of the wild-type strain, with minimal effect on evolved strains.

Based on these results, many of the causative mutations in evolved strains can be involved in either improving intracellular methionine supply, or allowing the cells to grow despite a condition of methionine starvation. This is clearly the case for loss-of-function mutations in metJ, which encodes a transcriptional repressor (MetJ) of methionine biosynthesis and transport genes and acts when it binds S-adenosyl-L-methionine (SAM), for which L-methionine is a precursor in the SAM cycle. Inactivating mutations in metJ have previously been seen to result in increased biosynthesis of methionine (Nakamori et al., 1999).

TABLE 20
Growth rates and lag times of the wild-type strain, selected
2,3-butanediol evolved strains, and K-12 MG1655 metJ::kan
in M9 supplemented with 0.3 g/L methionine, 6% (v/v) 2,3-
butanediol, or 6% (v/v) 2,3-butanediol and 0.3 g/L methionine,
as measured in the Biolector testing format.

| | M9 + methionine | | | | M9 + 23BDO | | | |
|---|---|---|---|---|---|---|---|---|
| | mean | | std. error | | Mean | | std. error | |
| | μ | t_{lag} | μ | t_{lag} | μ | t_{lag} | μ | t_{lag} |
| Strain | (h⁻¹) | (h) | (h⁻¹) | (h) | (h⁻¹) | (h) | (h⁻¹) | (h) |
| MG1655 | 0.805 | 1.3 | 0.033 | 0.2 | 0.194 | 9.3 | 0.010 | 1.0 |
| 23BD2-4 | 0.726 | 1.9 | 0.020 | 0.1 | 0.393 | 2.4 | 0.063 | 1.0 |
| 23BD6-1 | 0.997 | 2.5 | 0.034 | 0.2 | 0.541 | 4.7 | 0.005 | 0.3 |
| 23BD7-4 | 0.741 | 1.2 | 0.011 | 0.1 | 0.572 | 3.4 | 0.034 | 0.5 |
| MG1655 metJ::kan | 0.466 | 1.0 | 0.011 | 0.1 | 0.211 | 3.8 | 0.017 | 0.7 |

| M9 + 23BDO + methionine | |
|---|---|
| mean | std. error |

| | μ | t_{lag} | μ | t_{lag} |
|---|---|---|---|---|
| strain | (h⁻¹) | (h) | (h⁻¹) | (h) |
| MG1655 | 0.381 | 2.2 | 0.005 | 0.3 |
| 23BD2-4 | 0.345 | 1.0 | 0.031 | 0.7 |
| 23BD6-1 | 0.578 | 4.9 | 0.078 | 1.1 |
| 23BD7-4 | 0.609 | 4.0 | 0.023 | 0.2 |
| MG1655 metJ::kan | 0.202 | −0.7 | 0.021 | 1.1 |

TABLE 21
Growth rates and lag times of the wild-type strain and selected
1,2-propanediol evolved strains in M9 supplemented 8% (v/v)
1,2-propanediol, or 8% (v/v) 1,2-propanediol and 0.3 g/L methionine,
as measured in the Biolector testing format.

| | M9 + 12PDO | | | | | | | |
|---|---|---|---|---|---|---|---|---|
| | phase 1 | | | | phase 2 | | | |
| | mean | | std. error | | Mean | | std. error | |
| | μ | t_{lag} | μ | t_{lag} | μ | t_{lag} | μ | t_{lag} |
| Strain | (h⁻¹) | (h) | (h⁻¹) | (h) | (h⁻¹) | (h) | (h⁻¹) | (h) |
| MG1655 | 0.321 | 5.3 | 0.042 | 0.5 | 0.394 | 10.8 | 0.024 | 0.1 |
| 12PD3-8 | 0.523 | 19.0 | 0.037 | 0.7 | 0.282 | 20.9 | 0.036 | 1.7 |
| 12PD4-6 | 0.481 | 25.9 | 0.015 | 1.6 | — | — | — | — |
| 12PD6-3 | 0.470 | 1.8 | 0.057 | 1.2 | 0.780 | 11.7 | 0.094 | 0.6 |
| 12PD7-6 | 0.474 | 12.8 | 0.009 | 1.9 | — | — | — | — |
| | M9 + 12PDO + methionine | | | | | | | |
| | phase 1 | | | | phase 2 | | | |
| | mean | | std. error | | Mean | | std. error | |
| | μ | t_{lag} | μ | t_{lag} | μ | t_{lag} | μ | t_{lag} |
| Strain | (h⁻¹) | (h) | (h⁻¹) | (h) | (h⁻¹) | (h) | (h⁻¹) | (h) |
| MG1655 | 0.441 | 3.1 | 0.053 | 0.2 | 0.810 | 8.5 | 0.026 | 0.3 |
| 12PD3-8 | 0.344 | 27.8 | 0.053 | 3.9 | 0.427 | 35.7 | 0.100 | 4.4 |
| 12PD4-6 | 0.323 | 18.5 | 0.125 | 1.9 | — | — | — | — |
| 12PD6-3 | 0.473 | 1.5 | 0.024 | 1.4 | — | — | — | — |
| 12PD7-6 | 0.492 | 5.5 | 0.106 | 0.5 | — | — | — | — |

For the case of ethanol toxicity in E. coli, it was postulated that methionine starvation could be responsible for the observed ribosome stalling at non-start AUG codons, at which methionine is incorporated into translating proteins (Haft et al., 2014). Additionally, the stringent response alarmone guanosine tetraphosphate/guanosine pentaphosphate ((p)ppGpp) has been observed to accumulate as a consequence of growth in toxic concentrations of ethanol (Van Bogelen et al., 1987). (p)ppGpp is largely synthesized by RelA, which associates with with the ribosome and is activated by binding of uncharged tRNAs. (p)ppGpp regulates numerous gene products required for cell growth, with the net effect being the induction of a growth arrest (stringent response) when (p)ppGpp accumulates. If the toxicity mechanism of diols is similar to that of ethanol, then it would be expected that (p)ppGpp also accumulates in diol-stressed cells, and that this occurs via either the sensing of uncharged tRNAs in general by RelA (Hauryiuk et al., 2015), or detection of ribosome stalling by RelA due to lack of methionyl-tRNAs (Haft et al., 2014), or indirectly due to iron starvation (Miethke et al., 2006; Vinella et al., 2005) induced by toxic concentrations of diols, as elaborated on below.

Loss-of-function of RelA, which would prevent cells from entering the stringent response, was found in both 2,3-butanediol and 1,2-propanediol evolved strains, providing a functional linkage between the metJ and relA mutations. However these two mutations by themselves abolished growth, and growth was only rescued further by the additional purT deletion. PurT is one of two transformylases in purine biosynthesis, with the other being PurN. PurT utilizes the formyl group from formate, whereas PurN utilizes the formyl group from formyltetrahydrofolate (formyl-THF), which is also the formyl donor for generating initiator formylmethionine-tRNA (tRNA^{fMet}) that is required for initiating translation of AUG start codons. So, without being limited by theory, by deleting purT, competition for the formyl-THF pool between purine biosynthesis and tRNA^{fMet} biosynthesis results in overall reduced levels of tRNA^{fMet}, and can enable the cells to better cope with methionine starvation by having a more balanced ratio between initiator and non-initiator methionyl-tRNAs. This explanation provides a functional linkage between the metJ, relA, and purT genes that all involve coping strategies for methionine starvation, and could explain the negative epistasis in the metJ relA double knockout and the positive epistasis in the metJ relA purT triple knockout.

Methionine supplementation can thus be a strategy for improving endogenous production of diols in diol-overproducing strains during fermentation, since it is expected that growth would be inhibited by secreted diols at high concentrations due to the same mechanisms of toxicity observed here.

The combination of the presence of the iscR loss-of-function, which de-represses genes involved in iron-sulfur cluster biosynthesis when bound to free iron-sulfur clusters resulting from iron-sulfur protein degradation (Santos et al., 2015), in addition to the relA loss-of-function as well as SpoT coding mutations (present in some isolates) additionally indicates a role of modulation of levels of (p)ppGpp in relation to iron starvation. Iron starvation is known to trigger the stringent response and SpoT-dependent accumulation of (p)ppGpp in E. coli and other bacterial species (Miethke et al., 2006; Vinella et al., 2005), which is believed to help stimulate expression of iron uptake systems, thereby alleviating iron starvation conditions (Vinella et al., 2005). Thus the loss-of-function in relA, optionally in combination with a SpoT coding mutation such as SpoT-I213L or conservative substitutions thereof, may stimulate SpoT-dependent accumulation of (p)ppGpp and the increased expression of one or more iron uptake systems. Iron starvation could potentially arise from either direct chelation of iron by diols, or from diols interfering with chelation of iron by siderophores such as enterobactin. Derepression of iron-sulfur cluster biosynthesis and assembly enzymes via knockdown or knockout of iscR likely enables the more efficient use of cellular ferric iron for this critical function, as iron-sulfur clusters serve as catalytic cores of cytochromes involved in cellular respiration and in glutamate synthase. Furthermore, Miethke et al. (2006) speculated on the existence of a link between iron starvation and methionine and cysteine biosynthesis pathways, due to observance of up-regulation of several methionine and cysteine biosynthetic genes during iron starvation of B. subtilis. It was noted that in B. subtilis, L-threonine is a precursor for production of a catecholic trilactone siderophore that is utilized for ferric iron uptake, and that the threonine, serine/glycine, and cysteine/methionine biosynthetic pathways are interdependent. Conversely, in E. coli, L-serine is a precursor for the the production of enterobactin, another siderophore involved in ferric iron uptake. As L-cysteine is synthesized from L-serine, a reduction in levels of L-serine could lead to L-cysteine starvation and thus also L-methionine starvation, as L-cysteine is also a precursor for biosynthesis of L-methionine. Thus the combination of the metJ deletion and mutations that alleviate iron starvation, such as knockdown or knockout of relA and/or iscR, and optionally coding mutations in SpoT, may serve to restore cellular homeostasis at large.

Cross-Compound Tolerance Testing

Every secondary screened evolved isolate from the 2,3-butanediol and 1,2-propanediol evolutions was grown in the presence of every other compound in the study as indicated in the Methods. The normalized t_{OD1(evolved strain)}/t_{OD1(wild-type)} are shown in Table 21 (for 2,3-butanediol evolved strains) and Table 22 (for 1,2-propanediol evolved strains). Lower values are indicative a larger improvement in growth of the evolved isolate (left column) in that chemical condition (top row), whereas higher values are indicative of a lower improvement or decrease in growth compared to the wild-type. Averaged ratios across conditions and strains shown at the right and bottom of the plot allow for overall by-chemical and by-strain trends to be observed. Strain names that are followed by an asterisk (*) were not re-sequenced, and strain names in italics were found to be hypermutator strains.

All 2,3-butanediol evolved strains exhibit cross-tolerance to 1,2-propanediol, and isolates from populations 12PD5, 12PD6, 12PD7, and 12PD8, plus several isolates from the other populations, exhibit cross-tolerance to 2,3-butanediol. Isolates from populations 23BD1, 23BD8, 12PD5, 12PD6, and non-mutator isolates from 12PD8 all exhibit significant cross-tolerance to hexanoate, and 23BD8 isolates additionally have strong cross-tolerance to p-coumarate (this could be due to the mutation in ygaH having a pleiotropic effect on the neighboring mprA gene, which has been observed to improve p-coumarate tolerance when knocked out, or due to a broader effect from the mutation in rpoB). Several 12PD isolates also exhibit cross-tolerance toward coumarate, however the only non-hypermutator strain is 12PD6-9. This strain has non-coding mutations in ypjA, and mutations in ypjA thought to be inactivating were also found in p-coumarate evolved isolates. The 2,3-butanediol evolved strain with the best overall tolerance toward the range of chemical stressors was 23BD8-7. The majority of isolates being hypermutators was likely responsible for highly variable cross-tolerance between compounds in the 1,2-propanediol evolved strains, however the best-performing isolate was 12PD4-9.

TABLE 21
Normalized t_{OD1(evolved)}/t_{OD1(wild-type)} values for 2,3-butanediol-evolved isolates
grown in the presence of inhibitory concentrations of 12 different chemicals.

| | | | | 2,3- | pu- | | | iso- | | | 1,2- | | aver- |
|---|---|---|---|---|---|---|---|---|---|---|---|---|---|
| | butanol | glutarate | coumarate | butanediol | trescine | HMDA | adipate | butyrate | Hexanoate | octanoate | propanediol | NaCl | age |
| 23BD1-6 | 0.71 | 0.77 | 1.00 | 2.05 | 1.34 | 3.00 | 0.82 | 2.88 | 0.72 | 1.00 | 0.80 | 1.06 | 1.35 |
| 23BD1-8* | 0.98 | 1.89 | 1.00 | 0.50 | 1.26 | 1.50 | 0.80 | 2.87 | 0.76 | 1.00 | 0.73 | 1.21 | 1.21 |
| 23BD1-9 | 0.94 | 0.89 | 1.00 | 0.52 | 1.21 | 1.78 | 0.90 | 2.80 | 0.74 | 1.11 | 0.75 | 1.15 | 1.15 |
| 23BD2-4 | 1.20 | 1.89 | 1.00 | 2.05 | 1.67 | 1.06 | 2.03 | 1.80 | 1.09 | 1.70 | 0.89 | 1.75 | 1.51 |
| 23BD2-5* | 0.86 | 1.89 | 1.00 | 0.53 | 1.48 | 1.14 | 2.03 | 1.64 | 1.09 | 1.50 | 0.86 | 1.60 | 1.30 |
| 23BD2-7 | 0.86 | 1.89 | 1.00 | 0.53 | 1.42 | 1.02 | 2.03 | 1.80 | 1.02 | 1.51 | 0.80 | 1.74 | 1.30 |
| 23BD2-9 | 0.80 | 1.89 | 1.00 | 1.74 | 1.58 | 1.12 | 2.03 | 2.01 | 1.09 | 1.54 | 0.80 | 2.05 | 1.47 |
| 23BD3-3 | 1.20 | 1.48 | 1.00 | 0.42 | 3.10 | 3.00 | 1.82 | 1.62 | 3.56 | 1.70 | 0.77 | 2.35 | 1.84 |
| 23BD3-4 | 1.04 | 1.35 | 1.00 | 0.46 | 3.10 | 3.00 | 2.01 | 3.04 | 3.56 | 1.70 | 0.80 | 2.35 | 1.95 |
| 23BD3-9 | 1.20 | 1.26 | 1.00 | 0.55 | 3.10 | 3.00 | 1.81 | 2.10 | 3.16 | 1.70 | 0.91 | 2.35 | 1.85 |
| 23BD4-3 | 0.96 | 0.89 | 1.00 | 0.50 | 1.16 | 1.16 | 0.87 | 3.43 | 0.98 | 1.17 | 0.80 | 1.13 | 1.17 |
| 23BD4-4 | 1.01 | 1.25 | 1.00 | 1.74 | 1.56 | 1.76 | 1.17 | 3.43 | 0.93 | 1.70 | 0.84 | 1.38 | 1.48 |
| 23BD4-7 | 1.12 | 1.55 | 1.00 | 0.52 | 1.82 | 1.76 | 2.03 | 2.55 | 1.46 | 1.70 | 0.91 | 1.91 | 1.53 |
| 23BD5-1 | 1.15 | 1.00 | 0.86 | 2.05 | 1.43 | 1.33 | 1.17 | 2.51 | 0.93 | 0.99 | 0.80 | 1.38 | 1.30 |
| 23BD5-7 | 1.16 | 1.14 | 0.99 | 0.51 | 1.42 | 1.37 | 1.22 | 2.30 | 0.82 | 0.98 | 0.80 | 1.38 | 1.17 |
| 23BD5-10 | 1.14 | 1.05 | 0.77 | 0.62 | 1.53 | 1.42 | 1.13 | 2.37 | 0.85 | 1.00 | 0.77 | 1.38 | 1.17 |
| 23BD6-1 | 1.12 | 1.03 | 1.00 | 0.61 | 1.61 | 1.05 | 0.96 | 3.43 | 0.94 | 0.89 | 0.77 | 1.35 | 1.23 |
| 23BD7-4 | 1.02 | 1.52 | 0.79 | 0.38 | 1.63 | 1.56 | 2.03 | 1.84 | 0.89 | 1.70 | 0.71 | 1.78 | 1.32 |
| 23BD7-5 | 1.06 | 1.33 | 0.58 | 0.44 | 1.39 | 1.16 | 1.19 | 1.64 | 0.82 | 1.00 | 0.73 | 1.35 | 1.06 |
| 23BD7-7 | 0.99 | 1.43 | 1.00 | 0.38 | 1.56 | 1.58 | 2.03 | 1.85 | 0.80 | 1.70 | 0.73 | 1.80 | 1.32 |
| 23BD7-10* | 0.95 | 1.31 | 1.00 | 0.49 | 1.67 | 1.51 | 2.03 | 2.07 | 0.93 | 1.70 | 0.71 | 1.60 | 1.33 |
| 23BD8-2 | 1.20 | 0.84 | 0.55 | 0.58 | 1.79 | 3.00 | 1.01 | 0.82 | 0.67 | 0.94 | 0.77 | 1.53 | 1.14 |
| 23BD8-5* | 0.83 | 0.93 | 0.62 | 0.60 | 1.87 | 1.40 | 1.17 | 1.84 | 0.71 | 1.00 | 0.77 | 1.63 | 1.11 |
| 23BD8-7 | 0.77 | 0.74 | 0.30 | 0.47 | 1.21 | 0.91 | 0.71 | 1.62 | 0.67 | 0.89 | 0.73 | 0.82 | 0.82 |
| Average | 1.01 | 1.30 | 0.89 | 0.80 | 1.70 | 1.69 | 1.46 | 2.26 | 1.22 | 1.33 | 0.79 | 1.58 | 1.34 |
| # > wt | 11 | 6 | 8 | 19 | 0 | 1 | 6 | 1 | 16 | 5 | 24 | 1 | |
| % > wt | 45.8 | 25.0 | 33.3 | 79.2 | 0.0 | 4.2 | 25.0 | 4.2 | 66.7 | 20.8 | 100.0 | 4.2 | |

TABLE 22
Normalized t_{OD1(evolved)}/t_{OD1(wild-type)} values for 1,2-propanediol-evolved isolates
grown in the presence of inhibitory concentrations of 12 different chemicals.

| | | | | 2,3- | pu- | | | iso- | | | 1,2- | | aver- |
|---|---|---|---|---|---|---|---|---|---|---|---|---|---|
| | Butanol | glutarate | coumarate | butanediol | trescine | HMDA | adipate | butyrate | hexanoate | octanoate | propanediol | NaCl | age |
| 12PD1-2 | 0.94 | 1.42 | 1.42 | 1.86 | 3.06 | 2.84 | 1.52 | 2.14 | 0.98 | 1.44 | 1.15 | 2.24 | 1.75 |
| 12PD1-4 | 1.14 | 1.18 | 1.18 | 0.46 | 3.06 | 2.57 | 0.94 | 2.97 | 0.72 | 1.44 | 0.72 | 2.24 | 1.55 |
| 12PD1-10 | 1.26 | 1.36 | 1.36 | 0.71 | 3.06 | 2.84 | 1.56 | 1.63 | 1.51 | 1.44 | 1.15 | 2.24 | 1.68 |
| 12PD2-5* | 1.26 | 1.42 | 1.42 | 1.86 | 3.06 | 2.84 | 1.51 | 2.55 | 0.77 | 1.44 | 0.89 | 2.24 | 1.77 |
| 12PD2-8 | 1.26 | 1.52 | 1.52 | 0.62 | 3.06 | 2.84 | 1.48 | 2.77 | 1.15 | 1.44 | 1.00 | 2.24 | 1.74 |
| 12PD2-9 | 1.26 | 1.02 | 1.02 | 1.86 | 3.06 | 2.84 | 1.27 | 2.32 | 0.72 | 1.44 | 0.76 | 2.24 | 1.65 |
| 12PD3-7 | 1.26 | 1.02 | 1.02 | 0.39 | 3.06 | 2.84 | 1.71 | 0.77 | 0.70 | 0.84 | 0.65 | 2.24 | 1.38 |
| 12PD3-8 | 1.20 | 1.20 | 1.20 | 1.86 | 3.06 | 2.84 | 1.39 | 2.02 | 1.70 | 1.44 | 1.15 | 2.24 | 1.77 |
| 12PD3-10 | 1.26 | 1.36 | 1.36 | 0.74 | 3.06 | 2.84 | 1.54 | 2.20 | 1.21 | 0.99 | 0.96 | 2.24 | 1.65 |
| 12PD4-6 | 1.22 | 1.50 | 1.50 | 0.64 | 3.06 | 2.57 | 1.61 | 2.97 | 1.02 | 0.75 | 1.22 | 2.24 | 1.69 |
| 12PD4-8 | 1.12 | 0.83 | 0.83 | 1.86 | 1.44 | 0.99 | 0.76 | 0.72 | 0.91 | 0.75 | 0.65 | 0.80 | 0.97 |
| 12PD4-9 | 1.20 | 0.77 | 0.77 | 0.40 | 1.17 | 0.94 | 0.73 | 0.80 | 0.83 | 1.08 | 0.70 | 0.84 | 0.85 |
| 12PD5-1 | 1.26 | 1.84 | 1.84 | 0.41 | 3.06 | 2.84 | 1.70 | 2.97 | 0.72 | 1.01 | 0.59 | 2.24 | 1.71 |
| 12PD5-3 | 1.26 | 1.84 | 1.84 | 0.31 | 3.06 | 2.84 | 1.61 | 2.97 | 0.66 | 1.44 | 0.59 | 2.24 | 1.72 |
| 12PD5-9* | 1.26 | 1.84 | 1.84 | 0.41 | 3.06 | 2.84 | 1.46 | 1.08 | 0.70 | 0.80 | 0.57 | 2.24 | 1.51 |
| 12PD6-3 | 1.26 | 1.03 | 1.03 | 0.38 | 2.65 | 2.84 | 0.92 | 2.97 | 0.66 | 1.44 | 0.57 | 1.97 | 1.48 |
| 12PD6-6* | 1.26 | 0.73 | 0.73 | 0.41 | 2.29 | 2.53 | 0.76 | 2.97 | 0.66 | 0.73 | 0.63 | 1.62 | 1.28 |
| 12PD6-9 | 1.08 | 0.86 | 0.86 | 0.53 | 2.71 | 2.29 | 0.82 | 2.97 | 0.66 | 0.88 | 0.63 | 1.36 | 1.30 |
| 12PD7-5 | 1.15 | 0.59 | 0.59 | 0.35 | 1.17 | 1.12 | 0.56 | 2.97 | 0.70 | 0.71 | 0.59 | 0.66 | 0.93 |
| 12PD7-6 | 1.26 | 0.81 | 0.81 | 0.33 | 3.06 | 0.97 | 0.92 | 1.14 | 3.64 | 1.44 | 0.54 | 0.91 | 1.32 |
| 12PD7-7* | 1.18 | 1.30 | 1.34 | 0.34 | 1.76 | 1.09 | 1.87 | 1.02 | 0.74 | 1.44 | 0.59 | 1.72 | 1.20 |
| 12PD8-6 | 1.26 | 1.57 | 1.57 | 0.56 | 3.06 | 2.84 | 1.61 | 2.82 | 1.21 | 1.44 | 0.76 | 2.24 | 1.75 |
| 12PD8-7 | 1.26 | 0.73 | 0.73 | 0.31 | 3.06 | 2.34 | 0.68 | 1.89 | 0.49 | 0.84 | 0.54 | 1.14 | 1.17 |
| 12PD8-10 | 1.26 | 0.55 | 0.55 | 0.35 | 3.06 | 2.84 | 0.67 | 1.46 | 0.58 | 0.75 | 0.59 | 1.78 | 1.20 |
| average | 1.21 | 1.18 | 1.18 | 0.75 | 2.72 | 2.38 | 1.23 | 2.13 | 0.98 | 1.14 | 0.76 | 1.84 | 1.46 |
| # > wt | 1 | 8 | 8 | 19 | 0 | 3 | 10 | 3 | 17 | 10 | 19 | 4 | |
| % > wt | 4.2 | 33.3 | 33.3 | 79.2 | 0.0 | 12.5 | 41.7 | 12.5 | 70.8 | 41.7 | 79.2 | 16.7 | |

Additionally, each evolved isolate was tested for cross-tolerance toward other aliphatic diols of potential biotechnological interest. First, K-12 MG1655 was tested in the Growth Profiler screening format for growth in the presence of a range of concentrations of each compound (note that this had been done in the Biolector format previously for 1,2-pentanediol and 1,5-pentanediol thus was not repeated here): 1,3-propanediol and 1,4-butanediol. Variable concentrations of these compounds elicited growth inhibition in E. coli K-12 MG1655 (Table 23). Based on these results, a screening concentration was selected for the evolved isolates for which wild-type cells could achieve at a growth rate of 0.15-0.3 h⁻¹ (versus uninhibited growth at 0.7-0.9 h⁻¹ in M9 glucose minimal medium). These concentrations were: 5.5% (v/v) 1,3-propanediol, 5.5% (v/v) 1,4-butanediol, 1.25% (v/v) 1,2-pentanediol, and 3.5% (v/v) 1,5-pentanediol. The results of 2,3-butanediol-evolved isolates grown in these concentrations of alternative diols are shown in Table 24. All evolved isolates exhibited marked reductions in lag time in all tested diols. Additionally, all evolved isolates exhibited increased growth rates in 1,3-propanediol, 1,4-butanediol, and 1,5-pentanediol. Smaller numbers of isolates exhibited significantly improved growth rates in 1,2-pentanediol, however this included 23BD3-3, isolates from population 23BD4, 23BD6-1, isolates from population 23BD7 (with the most notable tolerance observed in 23BD7-5), and 23BD8-5 and 23BD8-7.

TABLE 23
Growth rates and lag times of K-12 MG1655 in varying
concentrations of 1,3-propanediol and 1,4-butanediol,
as measured in the Growth Profiler testing format.

| | 1,3-propanediol | | | | 1,4-butanediol | | | |
|---|---|---|---|---|---|---|---|---|
| | mean | | std. error | | mean | | std. error | |
| diol % | μ | t_{lag} | μ | t_{lag} | μ | t_{lag} | μ | t_{lag} |
| (v/v) | (h⁻¹) | (h) | (h⁻¹) | (h) | (h⁻¹) | (h) | (h⁻¹) | (h) |
| 0 | 0.747 | 5.1 | 0.030 | 0.2 | 0.747 | 5.1 | 0.030 | 0.2 |
| 1 | 0.670 | 5.3 | 0.093 | 0.1 | 0.672 | 5.3 | 0.018 | 0.1 |
| 2 | 0.686 | 6.1 | 0.010 | 0.3 | 0.591 | 5.9 | 0.024 | 0.1 |
| 3 | 0.603 | 7.3 | 0.057 | 0.5 | 0.531 | 7.1 | 0.033 | 0.1 |
| 4 | 0.463 | 9.6 | 0.037 | 0.6 | 0.434 | 9.0 | 0.009 | 0.2 |
| 5 | 0.277 | 12.5 | 0.002 | 0.6 | 0.303 | 11.7 | 0.042 | 0.2 |
| 6 | 0.201 | 14.7 | 0.012 | 0.7 | 0.183 | 17.6 | 0.000 | 0.4 |
| 7.5 | 0.118 | 24.9 | 0.029 | 0.6 | — | — | — | — |

TABLE 24
Growth rates and lag times of K-12 MG1655 and 2,3-butanediol-evolved isolates in specified
inhibitory concentrations of diols, as measured in the Growth Profiler testing format.

| | 5.5% (v/v) | | | | 5.5% (v/v) | | | | 1.25% (v/v) | | | | 3.5% (v/v) | | | |
|---|---|---|---|---|---|---|---|---|---|---|---|---|---|---|---|---|
| | 1,3-propanediol | | | | 1,4-butanediol | | | | 1,2-pentanediol | | | | 1,5-pentanediol | | | |
| | mean | | std. error | | mean | | std. error | | mean | | std. error | | mean | | std. error | |
| | (2) | | (2) | | (2) | | (2) | | (2) | | (2) | | (2) | | (2) | |
| | μ | t_{lag} | μ | t_{lag} | μ | t_{lag} | μ | t_{lag} | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) | (h⁻¹) | (h) | (h⁻¹) | (h) | (h⁻¹) | (h) | (h⁻¹) | (h) |
| MG1655 | 0.395 | 11.7 | 0.020 | 0.2 | 0.192 | 13.2 | 0.001 | 0.2 | 0.366 | 28.1 | 0.011 | 0.9 | 0.247 | 17.1 | 0.008 | 0.4 |
| 23BD1-6 | 0.639 | 6.2 | 0.020 | 0.1 | 0.501 | 6.9 | 0.011 | 0.1 | 0.336 | 9.6 | 0.018 | 0.7 | 0.384 | 10.5 | 0.011 | 1.8 |
| 23BD1-8 | 0.586 | 6.3 | 0.011 | 0.0 | 0.440 | 6.7 | 0.016 | 0.2 | 0.341 | 10.1 | 0.015 | 1.0 | 0.315 | 10.2 | 0.021 | 0.1 |
| 23BD1-9 | 0.578 | 6.4 | 0.013 | 0.1 | 0.471 | 7.0 | 0.013 | 0.1 | 0.314 | 9.4 | 0.045 | 0.0 | 0.326 | 10.7 | 0.007 | 1.1 |
| 23BD2-4 | 0.500 | 7.6 | 0.017 | 0.1 | 0.414 | 8.0 | 0.011 | 0.2 | 0.338 | 10.3 | 0.031 | 0.3 | 0.352 | 9.8 | 0.028 | 0.2 |
| 23BD2-7 | 0.529 | 7.2 | 0.015 | 0.1 | 0.418 | 7.3 | 0.004 | 0.0 | 0.367 | 9.7 | 0.030 | 0.4 | 0.315 | 14.0 | 0.014 | 1.3 |
| 23BD2-9 | 0.514 | 7.3 | 0.015 | 0.3 | 0.427 | 7.5 | 0.012 | 0.3 | 0.377 | 9.0 | 0.030 | 0.3 | 0.361 | 9.5 | 0.005 | 0.2 |
| 23BD3-3 | 0.647 | 6.4 | 0.012 | 0.2 | 0.565 | 6.8 | 0.009 | 0.2 | 0.422 | 8.0 | 0.006 | 0.4 | 0.379 | 8.6 | 0.010 | 0.2 |
| 23BD3-4 | 0.589 | 8.0 | 0.011 | 1.6 | 0.361 | 7.8 | 0.049 | 0.3 | 0.361 | 9.9 | 0.078 | 3.2 | 0.413 | 8.1 | 0.012 | 0.1 |
| 23BD3-9 | 0.681 | 7.2 | 0.008 | 0.3 | 0.523 | 7.6 | 0.026 | 0.2 | 0.409 | 8.7 | 0.023 | 0.9 | 0.279 | 10.1 | 0.015 | 0.7 |
| 23BD4-3 | 0.646 | 6.1 | 0.023 | 0.2 | 0.510 | 6.7 | 0.047 | 0.3 | 0.417 | 7.2 | 0.005 | 1.1 | 0.315 | 10.2 | 0.004 | 0.3 |
| 23BD4-4 | 0.644 | 6.5 | 0.013 | 0.6 | 0.508 | 7.8 | 0.046 | 1.0 | 0.459 | 8.5 | 0.015 | 2.5 | 0.373 | 8.4 | 0.024 | 0.7 |
| 23BD4-7 | 0.626 | 7.7 | 0.019 | 0.1 | 0.477 | 8.3 | 0.013 | 0.8 | 0.443 | 10.6 | 0.024 | 0.3 | 0.334 | 10.2 | 0.022 | 0.1 |
| 23BD5-1 | 0.651 | 6.8 | 0.018 | 0.2 | 0.474 | 7.9 | 0.013 | 0.1 | 0.340 | 9.9 | 0.010 | 0.8 | 0.380 | 8.1 | 0.004 | 0.1 |
| 23BD5-7 | 0.658 | 6.3 | 0.013 | 0.3 | 0.499 | 7.4 | 0.032 | 0.3 | 0.366 | 9.8 | 0.018 | 0.5 | 0.387 | 8.0 | 0.001 | 0.1 |
| 23BD5-10 | 0.666 | 6.4 | 0.019 | 0.2 | 0.524 | 7.7 | 0.007 | 0.3 | 0.348 | 9.7 | 0.021 | 0.7 | 0.392 | 8.2 | 0.014 | 0.0 |
| 23BD6-1 | 0.656 | 6.0 | 0.025 | 0.2 | 0.515 | 6.6 | 0.023 | 0.0 | 0.443 | 9.0 | 0.021 | 0.4 | 0.390 | 8.2 | 0.012 | 0.3 |
| 23BD2-5 | 0.520 | 6.7 | 0.005 | 0.6 | 0.437 | 7.2 | 0.010 | 0.2 | 0.357 | 9.6 | 0.027 | 0.3 | 0.351 | 9.1 | 0.009 | 0.2 |
| 23BD7-10 | 0.609 | 5.7 | 0.031 | 0.3 | 0.473 | 6.4 | 0.017 | 0.2 | 0.453 | 11.0 | 0.012 | 0.8 | 0.390 | 12.0 | 0.014 | 3.1 |
| 23BD7-4 | 0.617 | 5.9 | 0.018 | 0.1 | 0.481 | 6.5 | 0.005 | 0.1 | 0.462 | 11.1 | 0.011 | 0.1 | 0.310 | 10.0 | 0.017 | 0.4 |
| 23BD7-5 | 0.620 | 6.0 | 0.009 | 0.0 | 0.503 | 6.5 | 0.008 | 0.1 | 0.535 | 9.3 | 0.013 | 0.5 | 0.322 | 9.4 | 0.005 | 0.3 |
| 23BD7-7 | 0.629 | 5.7 | 0.012 | 0.1 | 0.497 | 6.3 | 0.020 | 0.1 | 0.461 | 10.8 | 0.008 | 0.2 | 0.316 | 10.8 | 0.033 | 0.5 |
| 23BD8-2 | 0.714 | 6.1 | 0.029 | 0.2 | 0.470 | 7.7 | 0.007 | 0.3 | 0.225 | 12.4 | 0.086 | 1.5 | 0.412 | 9.6 | 0.010 | 0.1 |
| 23BD8-5 | 0.625 | 5.6 | 0.061 | 0.2 | 0.434 | 6.7 | 0.071 | 0.3 | 0.493 | 5.5 | 0.043 | 0.3 | 0.411 | 9.6 | 0.015 | 0.4 |
| 23BD8-7 | 0.623 | 5.4 | 0.009 | 0.2 | 0.415 | 6.8 | 0.066 | 0.4 | 0.486 | 7.4 | 0.010 | 0.5 | 0.424 | 9.9 | 0.010 | 0.1 |

Biological Production of 1,2-Propanediol and 2,3-Butanediol

Known biological pathways for the production of 1,2-propanediol (S or R isomers) from various sugars or glycerol are shown in FIG. 8 of Dabra et al. (2016), hereby specifically incorporated by reference, where the pathway on the left is native to E. coli. In one pathway, the sugars L-rhamnose or L-fucose are catabolized to (S)-lactaldehyde and the glycolytic intermediate dihydroxyacetone phosphate (DHAP). Depending on redox conditions, S-lactaldehyde can either be oxidized to lactic acid, or reduced to (S)-1,2-propanediol. Another pathway, which is much more versatile in that any carbon feedstock can be utilized where DHAP can be readily generated (e.g. glucose, glycerol, or xylose), involves a methylglyoxal intermediate, which depending on the choice of reducing enzyme, can generate either (S)- or (R)-lactaldehyde, or acetol. These can then be further reduced to (S)- or (R)-1,2-propanediol, or acetol can be reduced to a racemic mixture of both isomers. The highest reported titer of 1,2-propanediol in E. coli is 5.6 g/L, and this was obtained using glycerol as a carbon source in a strain with inactivations of ackA-pta (acetate formation), replacement of the native PEP-dependent dihydroxyacetone kinase with an ATP-dependent enzyme from Citrobacter freundii, and overexpression of native methylglyoxal synthase (MgsA), L-1,2-propanediol dehydrogenase (GIdA), and NADPH-dependent aldehyde reductase YqhD (Clomburg et al., 2011). The highest reported titer of (R)-1,2-propanediol from glucose in E. coli is 5.13 g/L, obtained using a similar strategy aimed at improving DHAP availability and overexpressing a combination of native genes to covert DHAP to methylglyoxal (MgsA) and subsequently to lactaldehyde (GIdA) and 1,2-propanediol (FucO) (Jain et al., 2015). This pathway is shown in FIG. 1 of Jain et al. (2015), which is hereby specifically incorporated by reference in its entirety. Various alternative production pathways for 1,2-propanediol, either natively in natural 1,2-propanediol fermenting microorganisms or in recombinant strains with different combinations of enzymes, producing different enantiomers, and utilizing different carbon feedstocks, are known in the art.

Biological pathways for production of 2,3-butanediol in bacteria from glucose, CO2, and CO are shown in FIG. 2 of Sabra et al. (2016), which is hereby specifically incorporated by reference in its entirety. Generally (R)-acetoin is produced from acetolactate, however it is possible to isomerize acetoin to the (S)-isomer or to produce either isomer from diacetyl. Different acetoin reductases can then be utilized to generate 2,3-butanediol stereoisomers, or diacetyl can be used to generate acetylacetoin, from which different stereoisomers of 2,3-butanediol can ultimately derive. Many organisms natively ferment 2,3-butanediol, however most organisms are pathogenic and are not generally recognized as safe. Up to 119 g/L of 2,3-butanediol has been produced in Enterobacter cloacae subsp. dissolvens SDM utilizing lignocellulosic hydrolysates by simply deleting byproduct producing genes (Li et al., 2015). The best demonstrated production in recombinant E. coli from glucose is 73.8 g/L using E. coli BL21(DE3) containing a plasmid (pET-RABC) overexpressing a gene cluster from Enterobacter cloacae subsp. dissolvens SDM (lysR-budABC) with no other modifications (Xu et al., 2014). This is well above the toxicity threshold for this chemical (Table 1; 7.5% (v/v)=74.0 g/L), and a relatively low cell density was reached during their fed-batch fermentation despite continued production from glucose after cessation of cell growth (Xu et al., 2014), suggesting that higher productivities could be reached by increasing biomass through the use of evolved strains.

Endogenous production of 2,3-butanediol using the overproduction pathway described by Xu et al., 2014, was achieved by transforming plasmid pET-RABC into evolved isolates, plus wild-type K-12 MG1655 as a control, that all harbored inactivation of the EcoKI restriction system (due to the presence of restriction sites on the plasmid). Strains were cultured for production in a screening format as described in the Methods. It was found that cultures would not grow when harboring pET-RABC in a minimal medium without a complex nitrogen source, likely due to branched-chain amino acid starvation (e.g., isoleucine) due to introduction of the heterologous acetolactate synthase, thus yeast extract was utilized in the screen. Results are shown in Table 25. Perhaps unexpectedly due to the use of yeast extract, which was not employed in the evolutions, the majority of evolved isolates did not exhibit improved endogenous production of 2,3-butanediol as compared with wild-type K-12 MG1655 harboring the same modifications. However two isolates, 23BD7-5 and 23BD8-2, exhibited significantly increased titers of 2,3-butanediol, with up to a 67% improvement over the wild-type background. 23BD7-5 is notable in possessing a loss-of-function mutation in acre that the other isolates from population 23BD7 do not possess, however it also lacks mutations in tolC, treR, and yhjA that the other 23BD7 isolates possess. 23BD8-2 is notable in being the only resequenced evolved isolate lacking a mutation in metJ. It instead harbors a probable loss- or reduction-of-function mutation in iscR (inferred by Keio screening results described above) as well as mutations in relA, rpoB, Ion, ygaH, and a mutation that increases the expression of PyrE.

TABLE 25
2,3-butanediol titers in background strains harboring the ΔhsdR
mutation and plasmid pET-RABC, measured from screening in a
minimal medium containing 5% (w/v) glucose and 1% (w/v) yeast
extract after 48 hours at 30° C.

48 hour titer

| | average | std. error |
|---|---|---|
| background strain | (g/L) | (g/L) |
| K-12 MG1655 | 10.24 | 2.49 |
| 23BD1-6 | 9.48 | 0.34 |
| 23BD1-9 | 9.34 | 0.42 |
| 23BD2-4 | 5.54 | 0.77 |
| 23BD2-7 | 6.17 | 0.23 |
| 23BD2-9 | 5.60 | 0.19 |
| 23BD3-3 | 9.01 | 0.47 |
| 23BD3-4 | 7.92 | 2.13 |
| 23BD3-9 | 9.22 | 0.67 |
| 23BD4-3 | 9.10 | 0.05 |
| 23BD4-4 | 7.33 | 0.22 |
| 23BD4-7 | 9.34 | 0.21 |
| 23BD5-1 | 10.00 | 0.09 |
| 23BD5-7 | 10.09 | 0.35 |
| 23BD5-10 | 10.02 | 0.36 |
| 23BD6-1 | 6.75 | 0.08 |
| 23BD7-4 | 7.81 | 0.18 |
| 23BD7-5 | 17.12 | 0.12 |
| 23BD7-7 | 7.98 | 0.10 |
| 23BD8-2 | 13.14 | 0.34 |
| 23BD8-7 | 8.70 | 0.22 |

### LIST OF REFERENCES

Adkins J, Jordan J, Nielsen D R. Engineering Escherichia coli for renewable production of the 5-carbon polyamide building-blocks 5-aminovalerate and glutarate. Biotechnol. Bioeng. 110:1726-1734 (2013).
Altaras N E, Cameron D C. Enhanced production of (R)-1,2-propanediol by metabolically engineered Escherichia coli. Biotechnol. Prog. 16:940-946 (2000).
Baba T et al., Construction of Escherichia coli K-12 in-frame, single-gene knockout mutants: the Keio collection. Mol. Syst. Biol. 2:2006.0008 (2006).
Cheong S, Clomburg J M, Gonzalez R. Energy- and carbon-efficient synthesis of functionalized small molecules in bacteria using non-decarboxylative Claisen condensation reactions. Nat. Biotechnol. 34:556-561 (2016).
Clomburg J M, Gonzalez R. Metabolic engineering of Escherichia coli for the production of 1,2-propanediol from glycerol. Biotechnol. Bioeng. 108:867-879 (2011).
Conrad et al., Genome Biol. 10:R118 (2009).
Datsenko K A, Wanner B L. One-step inactivation of chromosomal genes in Escherichia coli K-12 using PCR products. Proc. Natl. Acad. Sci. USA 97:6640-6645 (2000).
Doukyu N, Ishikawa K, Watanabe R, Ogino H. Improvement in organic solvent tolerance by double disruption of proV and marR genes in Escherichia coli. J. Appl. Microbiol. 112:464-474 (2012).
Dragosits M, Mattanovich D. Adaptive laboratory evolution—principles and applications for biotechnology, Microbial Gell Factories 12:64 (2013).
Dragosits M, Mozhayskiy V, Quinones-Soto S, Park J, Tagkopoulos I. Evolutionary potential, cross-stress behavior and the genetic basis of acquired stress resistance in Escherichia coli. Mol. Syst. Biol. 9:643 (2013).
Grafje H, Körnig W, Weitz H-M, Reiss W, Steffan G, Diehl H, Bosche H, Schneider K, Kieczka H. “Butanediols, Butenediol, and Butynediol.” Ullmann's Encyclopedia of Industrial Chemistry. Wiley-VCH Verlag GmbH & Co. KGaA. Weinheim, Germany. 2012.
Haft R J F, et al. Correcting direct effects of ethanol on translation and transcription machinery confers ethanol tolerance in bacteria. Proc. Natl. Acad. Sci. USA 111:E2576-E2585 (2014).
Haft R J F, Keating D H, Schwaegler T, Schwalbach M S, Vinokur J, Tremaine M, Peters J M, Kotlajich M V, Pohlmann E L, Ong I M, Grass J A, Kiley P J, Landick R. Correcting direct effects of ethanol on translation and transcription machinery confers ethanol tolerance in bacteria. Proc. Natl. Acad. Sci USA 111:E2576-E2585 (2014).
Hauryliuk V, Atkinson G C, Murakami K S, Tenson T, Gerdes K. Recent functional insights into the role of (p)ppGpp in bacterial physiology. Nat. Rev. Microbiol. 13:298-309 (2015).
Hirschmann S, Koschik I, Baganz K, Vorlop K. Development of an integrated bioconversion process for the production of 1,3-propanediol from raw glycerol waters. Landbauforschung Völkenrode 55:261-267 (2005).
Jain R, Sun X, Yuan Q, Yan Y. Systematically engineering Escherichia coli for enhanced production of 1,2-propanediol and 1-propanol. ACS Synth. Biol. 4:746-756 (2015).
Jensen J V, Eberhardt D, Wendisch V F. Modular pathway engineering of Corynebacterium glutamicum for production of the glutamate-derived compounds ornithine, proline, putrescine, citrulline, and arginine. J. Biotechnol. 214:85-94 (2015).
Jensen S I, Lennen R M, Herrard M J, Nielsen A T. Seven deletions in seven days: Fast generation of Escherichia coli strains tolerant to acetate and osmotic stress. Sci. Rep., accepted.
Jiang Y, Liu W, Zou H, Cheng T, Tian N, Xian M. Microbial production of short chain diols. Microbial Cell Factories 13:165 (2014).
Kataoka N, Vangnai A S, Tajima T, Nakashimada Y, Kato J. Improvement of (R)-1,3-butanediol production by engineered Escherichia coli. J. Biosci. Bioeng. 115:475-480 (2013).
Kenyon S H, Nicolaou A, Gibbons W A. The effect of ethanol and its metabolites upon methionine synthase activity in vitro. Alcohol 15:305-309 (1998).
Köpnick H, Schmidt M, Brugging W, Rüter J, Kaminsky W. “Polyesters.” Ullmann's Encyclopedia of Industrial Chemistry. Wiley-VCH Verlag GmbH & Co. KGaA. Weinheim, Germany. 2012.
LaCroix R A, et al. Use of adaptive laboratory evolution to discover key mutations enabling rapid growth of Escherichia coli K-12 MG1655 on glucose minimal medium. Appl. Environ. Microbiol. 81:17-30 (2015).
Lennen R M, Herrard M J. Combinatorial strategies for improving multiple-stress resistance in industrially relevant Escherichia coli strains. Appl. Environ. Microbiol. 80:6223-6242 (2014).
Li L, Li K, Wang Y, Chen C, Xu Y, Zhang L Han B, Gao C, Tao F, Ma C. Metabolic engineering of Enterobacter cloacae for high-yield production of enantiopure (2R,3R)-2,3-butanediol from lignocellulose-derived sugars. Metab. Eng. 28:19-27 (2015).
Lieber C S. Mechanism of ethanol induced hepatic injury. Pharmac. Ther. 46:1-41 (1990).
Ma C, Wang A, Qin J, Li L, Ai X, Jiang T, Tang H, Xu P. Enhanced 2,3-butanediol production of Klebsiella pneumoniae SDM. Appl. Microbiol. Biotechnol. 82:49-57 (2009).
Miethke M, Westers H, Blom E J, Kuipers O P, Marahiel M A. Iron starvation triggers the stringent response and induces amino acid biosynthesis for bacillibactin production in Bacillus subtilis. J. Bacteriol. 188:8655-8657 (2006).
Minty J J, et al. Evolution combined with genomic study elucidates genetic bases of isobutanol tolerance in Escherichia coli. Microbial Cell Factories 10:18 (2011).
Nakamori S, Kobayashi S, Nishimura T, Takagi H. Mechanism of L-methionine overproduction in Escherichia coli: the replacement of Ser-54 by Asn in the MetJ protein causes the derepression of L-methionine biosynthetic enzymes. Appl. Microbiol. Biotechnol. 52:179-185 (1999).
Nakamura C E, Whited G M. Metabolic engineering for the microbial production of 1,3-propanediol. Curr. Opin. Biotechnol. 14:454-459 (2003).
Park S J, Kim E Y, Noh W, Park H M, Oh Y H, Lee S H, Song B K, Jegal J, Lee S Y. Metabolic engineering of Escherichia coli for the production of 5-aminovalerate and glutarate as C5 platform chemicals. Metab. Eng. 16:42-47 (2013).
Rath D, Jawali N. Loss of expression of cspC, a cold shock family gene, confers a gain of fitness in Escherichia coli K-12 strains. J. Bacteriol. 188:6780-6785 (2006).
Sabra W, Groeger C, Zeng A-P. Microbial cell factories for diol production. Adv. Biochem. Eng. Biotechnol. 155:165-197 (2016).
Sandberg T E, et al. Evolution of Escherichia coli to 42° C. and subsequent genetic engineering reveals adaptive mechanisms and novel mutations. Mol. Biol. Evol. 31:2647-2662 (2014).
Santos J A, Pereira P J B, Macedo-Ribeiro S. What a difference a cluster makes: The multifaceted roles of IscR in gene regulation and DNA recognition. Biochim. Biophys. Acta 1854:1101-1112 (2015).
Saxena R K, Anand P, Saran S, Isar J, Agarwal L. Microbial production and applications of 1,2-propanediol. Indian J. Microbiol. 50:2-11 (2010).
Shaw S, Jayatilleke E, Ross W A, Gordon E R, Leiber C S. Ethanol-induced lipid peroxidation: potentiation by long-term alcohol feeding and attenuation by methionine. J. Lab. Clin. Med. 98:417-424 (1981).
Shenhar Y, Biran D, Zon E Z. Resistance to environmental stress requires the RNA chaperones CspC and CspE. Environ. Microbiol. Rep. 4:532-539 (2012).
Sullivan C J. “Propanediols.” Ullmann's Encyclopedia of Industrial Chemistry. Wiley-VCH Verlag GmbH & Co. KGaA. Weinheim, Germany. 2012.
Tenaillon O, Rodriguez-Verdugo A, Gaut R L, McDonald P, Bennett A F, Long A D, Gaut B S. The molecular diversity of adaptive convergence. Science 335:457-461 (2012).
Thomason L, Court D L, Bubunenko M, Constantino N, Wilson H, Datta S, Oppenheim A. Recombineering: Genetic engineering in bacteria using homologous recombination. Curr. Protoc. Molec. Biol. 1.16 (2007).
Thomason L C, Constantino N, Court D L. E. coli genome manipulation by P1 transduction. Curr. Protoc. Molec. Biol. 1.17 (2007).
Van Bogelen R A, Kelley P M, Neidhardt F C. Differential induction of heat shock, SOS, and oxidation stress regulons and accumulation of nucleotides in Escherichia coli. J. Bacteriol. 169:26-32 (1987).
Van Dien S. From the first drop to the first truckload: commercialization of microbial processes for renewable chemicals. Gurr. Opin. Biotechno. 24:1-8 (2013).
Vinella D, Albrecht C, Cashel M, D'Ari R. Iron limitation induces SpoT-dependent accumulation of ppGpp in Escherichia coli. Molec. Microbiol. 56:958-970 (2005).
Werle P, Morawietz M, Lundmark S, Sörensen K, Karvinen E, Lehtonen J. “Alcohols, Polyhydric.” Ullmann's Encyclopedia of Industrial Chemistry. Wiley-VCH Verlag GmbH & Co. KGaA. Weinheim, Germany. 2012.
Winkler J D, et al. Evolved osmotolerant Escherichia coli mutants frequently exhibit defective Nacetylglucosamine catabolism and point mutations in the cell shape-regulating protein MreB. Appl. Environ. Microbiol. 80:3729-3740 (2014).
Wu X, Altman R, Eiteman M A, Altman E. Adaptation of Escherichia coli to elevated sodium concentrations increases cation tolerance and enables greater lactic acid production. Appl. Environ. Microbiol. 80:2880-2888 (2014).
Xu Y, Chu H, Gao C, Tao F, Zhou Z, Li K, Li L, Ma C, Xu P. Systematic metabolic engineering of Escherichia coli for high-yield production of fuel bio-chemical 2,3-butanediol. Metab. Eng. 23:22-33 (2014).
Yim H, et al. Metabolic engineering of Escherichia coli for direct production of 1,4-butanediol. Nat. Chem. Biol. 7:445-452 (2011).
Zhu L, Guan X, Xie N, Wang L, Yu B, Ma Y. Fermentative production of enantiomerically pure S-1,2-propanediol from glucose by engineered E. coli strain. Appl. Microbiol. Biotechnol. 100:1241-1251 (2016).
Zingaro K A, et al. Dissecting the assays to assess microbial tolerance to toxic chemicals in bioprocessing. Trends Biotechnol. 31:643-653 (2013).
US 2012/0282661 A1 (Genomatica Inc.)
WO 2009/086075 A1 and US 2009/0162911 A1 (E I Du Pont De Nemours and Company)
US 2003/0068611 A1 (E I DuPont De Nemours and Company)
WO 2009/143455 A2 (Microbia Inc.)
WO 2011/154503 A1 (Evonik Degussa GmbH)
US 2014/0135526 A1 (OPX Biotechnologies Inc.)
WO 2014/029592 A1 (Evonik Degussa GmbH)
EP 2 032 711 A1 (TNO)
EP 2 580 315 A2 (Cobalt Technologies Inc.)

1. A bacterial cell comprising a biosynthetic pathway for producing an aliphatic polyol and at least one genetic modification which reduces expression of an endogenous gene selected from the group consisting of metJ, iscR, yhjA, gtrS, ycdU, rzpD, sspA and rph, or a combination of any thereof, optionally wherein the cell further comprises a genetic modification which increases the expression of PyrE.

2. The bacterial cell of claim 1, comprising at least one genetic modification which reduces expression of metJ, iscR, or both.

3. A bacterial cell comprising at least one genetic modification which reduces expression of
(a) metJ, relA and purT;
(b) metJ and acrB, acrA or both;
(c) fabR and ygfF; or
(d) iscR and relA;
optionally in combination with a genetic modification which increases the expression of PyrE.

4. The bacterial cell of claim 1, wherein the genetic modification comprises a knock-down or knock-out of the endogenous gene or genes.

5. The bacterial cell of claim 1, further comprising an upregulation of, and/or one or more mutations in, at least one protein selected from NanK (SEQ ID NO:19), RpsA (SEQ ID NO:37), RpoA (SEQ ID NO:21); RpoB (SEQ ID NO:23), RpoC (SEQ ID NO:25), SpoT (SEQ ID NO:27), NusG (SEQ ID NO:29, Flu (SEQ ID NO:31), Lon (SEQ ID NO:33), and YgaH (SEQ ID NO:35), wherein the one or more mutations are selected from RpoC-L268K, RpoC-L268N, RpoC-L268Q, RpoC-L268R, RpoC-N309F, RpoC-N309S, RpoC-N309T, RpoC-N309W, RpoC-N309Y, RpoC-Y75A, RpoC-Y75C, RpoC-Y75S, RpoC-ΔTPVIE(822-827), RpoB-D549A, RpoB-D549G, RpoB-H447F, RpoB-H447S, RpoB-H447T, RpoB-H447W, RpoB-H447Y, RpoB-I1112S, RpoB-I1112T, RpoB-V931A, RpoB-V931I, RpoB-V931L, NanK-T128S, Flu-L642E, Flu-L642N, Flu-L642Q, Lon-1716S, Lon-1716T, YgaH-V39A, YgaH-V39I, YgaH-V39L, NusG-F144A, NusG-F144I, NusG-F144L, NusG-F144M, NusG-F144V, RpoA-D305A, RpoA-D305G, RpoA-G279A, RpoA-G279F, RpoA-G279I, RpoA-G279L, RpoA-G279M, RpoA-G279V, RpsA-D310A, RpsA-D310F, RpsA-D310I, RpsA-D310L, RpsA-D310M, RpsA-D310V, RpsA-G21A, RpsA-G21F, RpsA-G21I, RpsA-G21L, RpsA-G21M, RpsA-G21V, SpoT-I213A, SpoT-I213F, SpoT-I213L, SpoT-I213M, and SpoT-I213V.

6. The bacterial cell of claim 1, comprising
(a) a mutant RpoC comprising a RpoC-L268R, RpoC-L268K, RpoC-L268Q or RpoC-L268N mutation and at least one genetic modification which reduces the expression of metJ, relA and purT;
(b) a mutant RpoC comprising a RpoC-L268R, RpoC-L268K, RpoC-L268Q, or RpoC-L268N mutation and at least one genetic modification which reduces the expression of metJ and acrB, acrA or both;
(c) a mutant RpoC comprising a RpoC-L268R, RpoC-L268K, RpoC-L268Q or RpoC-L268N mutation and at least one genetic modification which reduces the expression of metJ, relA, purT, and acrB, acrA or both;
(d) a mutant RpoC comprising a RpoC-L268R, RpoC-L268K, RpoC-L268Q, or RpoC-L268N mutation and a mutant NanK comprising a NanK-T128S mutation, and at least one genetic modification which reduces the expression of metJ, relA, and purT, and acrB, acrA or both;
(e) a mutant RpoC comprising a RpoC-L268R, RpoC-L268K, RpoC-L268Q, or RpoC-L268N mutation and a mutant NanK comprising a NanK-T128S mutation, and at least one genetic modification which reduces the expression of metJ and acrB, acrA or both;
(f) a mutant RpoC comprising a RpoC-L268R, RpoC-L268K, RpoC-L268Q, or RpoC-L268N mutation and a mutant NanK comprising a NanK-T128S mutation, and at least one genetic modification which reduces the expression of metJ, relA, purT, and acrB, acrA or both;
(g) a mutant RpoC comprising a RpoC-L268R, RpoC-L268K, RpoC-L268Q or RpoC-L268N mutation, a mutant NanK comprising a NanK-T128S mutation, and a mutant Flu comprising a Flu-L642Q, Flu-L642N, or Flu-L642E mutation, and at least one genetic modification which reduces the expression of metJ, relA, purT, elfD and acrB, acrA or both;
(h) a mutant RpoB comprising a RpoB-I1112S or RpoB-I1112T mutation and at least one genetic modification which reduces the expression of iscR, relA, and acrB, acrA or both;
(i) a mutant RpoB comprising a RpoB-I1112S or RpoB-I1112T mutation and at least one genetic modification which reduces the expression of iscR, relA, and acrB, acrA or both;
(j) a mutant RpoB comprising a RpoB-I1112S or RpoB-I1112T mutation, and a mutant Lon comprising a Lon-1716S or Lon-1716T mutation, and at least one genetic modification which reduces the expression of iscR, relA, and acrB, acrA or both;
(k) a mutant RpoB comprising a RpoB-I1112S or RpoB-I1112T mutation, a mutant Lon comprising a Lon-1716S or Lon-1716T mutation, and a mutant YgaH comprising a YgaH-V39A, YgaH-V39L, or YgaH-V39I mutation, and at least one genetic modification which reduces the expression of iscR, relA, and acrB, acrA or both; or
(l) a mutant RpoB comprising a RpoB-I1112S or RpoB-I1112T mutation, a mutant Lon comprising a Lon-1716S or Lon-1716T mutation, a mutant YgaH comprising a YgaH-V39A, YgaH-V39L, or YgaH-V39I mutation, a genetic modification that increases the expression of PyrE, and at least one genetic modification which reduces the expression of iscR, relA, and acrB, acrA or both.

7. The bacterial cell of claim 1, wherein the at least one genetic modification provides for an increased growth rate, a reduced lag time, or both, of the cell in at least one of 2,3-butanediol and 1,2-propanediol, as compared to the parent bacterial cell.

8. The bacterial cell of claim 1, comprising a recombinant biosynthetic pathway for producing at least one of a propanediol, butanediol, pentanediol and a hexanediol.

9. The bacterial cell of claim 1, which is of the Escherichia, Enterobacter, Klebsiella, Lactobacillus, Lactococcus, Bacillus, Pseudomonas, Corynebacterium, Ralstonia, Paenibacillus, Clostridia or Citrobacter sp genera, such as of the Escherichia coli species.

10. A process for a bacterial cell according to claim 1, comprising genetically modifying an E. coli cell to introduce a recombinant biosynthetic pathway for producing an aliphatic polyol, and
(a) knock-down or knock-out at least one endogenous gene selected from the group consisting of metJ, iscR, yhjA, gtrS, ycdU, rzpD, sspA and rph; or a combination of endogenous genes selected from metJ, relA and purT; metJ and acrB, acrA or both; iscR and relA; and fabR and ygfF; and
(b) optionally, upregulating and/or introducing one or more mutations in at least one protein selected from NanK (SEQ ID NO:19), RpsA (SEQ ID NO:37), RpoA (SEQ ID NO:21); RpoB (SEQ ID NO:23), RpoC (SEQ ID NO:25), SpoT (SEQ ID NO:27), NusG (SEQ ID NO:29, Flu (SEQ ID NO:31), Lon (SEQ ID NO:33), and YgaH (SEQ ID NO:35), optionally wherein the one or more mutations are selected from RpoC-L268K, RpoC-L268N, RpoC-L268Q, RpoC-L268R, RpoC-N309F, RpoC-N309S, RpoC-N309T, RpoC-N309W, RpoC-N309Y, RpoC-Y75A, RpoC-Y75C, RpoC-Y75S, RpoC-ΔTPVIE(822-827), RpoB-D549A, RpoB-D549G, RpoB-H447F, RpoB-H447S, RpoB-H447T, RpoB-H447W, RpoB-H447Y, RpoB-I1112S, RpoB-I1112T, RpoB-V931A, RpoB-V931I, RpoB-V931L, NanK-T128S, Flu-L642E, Flu-L642N, Flu-L642Q, Lon-1716S, Lon-1716T, YgaH-V39A, YgaH-V39I, YgaH-V39L, NusG-F144A, NusG-F144I, NusG-F144L, NusG-F144M, NusG-F144V, RpoA-D305A, RpoA-D305G, RpoA-G279A, RpoA-G279F, RpoA-G279I, RpoA-G279L, RpoA-G279M, RpoA-G279V, RpsA-D310A, RpsA-D310F, RpsA-D310I, RpsA-D310L, RpsA-D310M, RpsA-D310V, RpsA-G21A, RpsA-G21F, RpsA-G21I, RpsA-G21L, RpsA-G21M, RpsA-G21V, SpoT-I213A, SpoT-I213F, SpoT-I213L, SpoT-I213M, and SpoT-I213V.

11. A process for improving the tolerance of a bacterial cell to an aliphatic polyol, comprising genetically modifying the bacterial cell to
(a) knock-down or knock-out at least one endogenous gene selected from the group consisting of metJ, iscR, yhjA, gtrS, ycdU, rzpD, sspA and rph; or a combination of endogenous genes selected from metJ, relA and purT; metJ and acrB, acrA or both; iscR and relA; and fabR and ygfF;
(b) optionally introducing one or more mutations in one or more endogenous genes selected from NanK (SEQ ID NO:19), RpsA (SEQ ID NO:37), RpoA (SEQ ID NO:21); RpoB (SEQ ID NO:23), RpoC (SEQ ID NO:25), SpoT (SEQ ID NO:27), NusG (SEQ ID NO:29, Flu (SEQ ID NO:31), Lon (SEQ ID NO:33), and YgaH (SEQ ID NO:35) or the pyrE/rph intergenic region;
(c) preparing a population of the genetically modified bacterial cell; and
(d) selecting from the population in (c) any bacterial cell which has an improved tolerance to the aliphatic polyol.

12. A method for producing an aliphatic polyol, comprising culturing the bacterial cell of claim 1, in the presence of a carbon source, and, optionally, isolating the aliphatic polyol.

13. A composition comprising a propanediol or a butanediol at a concentration of at least 6% v/v and a plurality of bacterial cells according to claim 1.

14. A bacterial cell comprising a biosynthetic pathway for producing an aliphatic polyol and at least one genetic modification which increases one or more of
(a) the biosynthesis of methionine in the bacterial cell;
(b) growth of the bacterial cell during polyol-induced methionine starvation;
(c) intracellular iron levels during polyol-induced growth inhibition;
(d) biosynthesis of iron siderophores during polyol-induced growth inhibition; and
(e) biosynthesis of iron-sulfur clusters during polyol-induced growth inhibition, wherein the bacterial cell is the bacterial cell of claim 1.

15. A method for producing an aliphatic diol, comprising
(a) culturing a plurality of bacterial cells capable of producing the aliphatic diol in a medium, the medium comprising methionine at a concentration of from about 0.004 g L⁻¹ gDCW⁻¹ to about 0.2 g L⁻¹gDCW⁻¹ and at least one carbon source, wherein the medium comprises no more than 4 other natural amino acids at a concentration of at least 0.002 g L⁻¹gDCW⁻¹; and
(b) optionally, isolating the aliphatic diol,
wherein the bacterial cell is the bacterial cell of claim 1.
